SEQUENCE LISTING (80 sequences)

SEQ ID NO: 1 - DNA, 405 bp, Homo sapiens
atgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtgagcgtctttggcagtaaagtgagaggggagaacaagctctccctcctctcctgccagaaggacgtcggattccccctgctgaaacttacaagtgccgttgtggactcccagatctcttgctgcaagggagacctctgcaatgcggtggtcctggcagccagcagcccctgggccctgtgcgtacagctcctgctcagcctggggtcagtcttcctctgggccctgctgtga

SEQ ID NO: 2 - protein, 134 aa, Homo sapiens
MSSLQAMKTLSLVLLVALLSMERAQGLRCYRCLAVLEGASCSVVSCPFLDGVCVSQKVSVFGSKVRGENKLSLLSCQKDVGFPLLKLTSAVVDSQISCCKGDLCNAVVLAASSPWALCVQLLLSLGSVFLWALL

SEQ ID NO: 3 - DNA, 345 bp, Homo sapiens
atgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaaaaggacgtcggattccccctgctgaaacttacaagtgccgttgtggactcccagatctcttgctgcaagggagacctctgcaatgcggtggtcctggcagccagcagcccctgggccctgtgcgtacagctcctgctcagcctggggtcagtcttcctctgggccctgctgtga

SEQ ID NO: 4 - protein, 114 aa, Homo sapiens
MSSLQAMKTLSLVLLVALLSMERAQGLRCYRCLAVLEGASCSVVSCPFLDGVCVSQKKDVGFPLLKLTSAVVDSQISCCKGDLCNAVVLAASSPWALCVQLLLSLGSVFLWALL

SEQ ID NO: 5 - DNA, 498 bp, Homo sapiens
ctgaagtttgtctgtgcactagcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtaaagtgggaggggagaacaagctccccctcctctcctgccagaaggacgtcggattccccctgctgaaacttacaagtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccagctgcttggttttgaagagctgc

SEQ ID NO: 6 - protein, 79 aa, Homo sapiens
MSSLQAMKTLSLVLLVALLSMERAQGLRCYRCLAVLEGASCSVVSCPFLDGVCVSQKVKWEGRTSSPSSPARRTSDSPC

SEQ ID NO: 7 - DNA, 494 bp, Homo sapiens
  misc_feature (27)..(27): n is a, c, g, or t
ctgaagtttgtctgtgcactccttaanctggaatgagcagtctccaggccatgaagaccttgtccctggtcctgctggtggccctgctgagcatggagagagggtctgcgctgctacagatgcttggcggtcttggaaggggcctcctgcagcgtggtctcgtgccccttcctggatggggtctgtgtctcccagaaagtaaagtgagaggggagaacaagctctccctcctctcctgccagaaggacgtcggattccccctgctgaaacttacaagtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccagctgcttggttttgaagagctgc

SEQ ID NO: 8 - protein, 127 aa, Homo sapiens
MSSLQAMKTLSLVLLVALLSMERGSALLQMLGGLGRGLLQRGLVPLPGWGLCLPESKVRGENKLSLLSCQKDVGFPLLKLTSAVVDSQISCCKGDLCNAVVLAAGSPWALCVQLLLSLGSVFLWALL

SEQ ID NO: 9 - DNA, 505 bp, Homo sapiens
  misc_feature (476)..(476): n is a, c, g, or t
ctgaagtttgtctgcgcactagcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtaaagtgagaggggagaacaagctctccctcctctcctgccagaaggacgtcggattccccctgctgaaacttacaggtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccanctgcttggttttgaagagctgcaatcgaa

SEQ ID NO: 10 - protein, 59 aa, Homo sapiens
MSSLQAMKTLSLVLLVALLSMERAQGLRCYRCLAVLEGASCSVVSCPFLDGVCVSQKVK

SEQ ID NO: 11 - DNA, 473 bp, Homo sapiens
ctgaagtttgtctgtgcactggcaccctggaatgagcagtctccaggccatggagaccttgtccctggtcctgctggtggccctgcttgccctctctcccagctcagggtctgcgctgctacagatgcttggcggtcttggaaggggcctcctgcagcgtggtctcgtgccccttcctggatggggtctgtgtctcccagaaagtgagcgtctttggcagtgagtccctggggtgccagggcagagggcaggttaagtgccgttgtggactcccagatctcttgctgcaagggagacctctgcaatgcggtggtcctggcagccggcagcccctgggccctgtgcgtacagctcctgctcagcctggggtcagtcttcctctgggccccgctgtgagggcctttcccgccctctcccccgcaggcctaccctctgtccctgtgcgtcaccagctgcttggttttgaagagctg

SEQ ID NO: 12 - protein, 63 aa, Homo sapiens
MSSLQAMETLSLVLLVALLALSPSSGSALLQMLGGLGRGLLQRGLVPLPGWGLCLPESERLWQ

SEQ ID NO: 13 - DNA, 443 bp, Homo sapiens
  misc_feature (398)..(398): n is a, c, g, or t
ctgaagtttgtctgtgcactagcaccctggaatgagcagtctccaggccatgaagaccttgtccctggtcctgctggtggcccttgccctctctcccagctcagggtctgcgctgctacagatgcttggcggtcttggaaggggcctcctgcagcgtggtctcgtgccccttcctggatggggtctgtgtctcccagaaagtgagcgtctttggcagtgagtccctggggtgccagggcagagggcaggttcagctccatgcaggagaggcgcaggctgtgagcattcagtgagttacctgcctggaagaacaagtgccgttgtggactcccagatctcttgctgcaagggagacctctgcaatgcggtggtcctggcagccagcagccccctctgtnccctgtgcgtcaccagctgcttggttttgaagagctgcaatcgaa

SEQ ID NO: 14 - protein, 62 aa, Homo sapiens
MSSLQAMKTLSLVLLVALALSPSSGSALLQMLGGLGRGLLQRGLVPLPGWGLCLPESERLWQ

SEQ ID NO: 15 - DNA, 515 bp, Homo sapiens
  misc_feature (478)..(478): n is a, c, g, or t
ctgaagtttgtctgtgcactagcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtgagcgtctttggcagtaaagtgagaggggagaacaagctctccctcctctcctgccagaaggacgtcggattccccctgctgaaacttacaagtgccgttgtggactcccagatctcttgctgcaagggagacctctgcaatgcggtggtcctggcagccagcagcccctgggccctgtgcgtacagctcctgctcagcctggggtcagtcttcctctgggccctgctgtgagggcctttcccgccctctcccccgcgggcctagccctctgtnccctgtgcgtcaccagctgcttggtttgaagagctgc

SEQ ID NO: 16 - protein, 134 aa, Homo sapiens; identical to SEQ ID NO: 2

SEQ ID NO: 17 - DNA, 512 bp, Homo sapiens
ctgaagtttgtctgtgcactagcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtgagcgtctttggcagtaaagtgagaggggagaacaagctctccctcctctcctgccagaaggacgtcggattccccctgctgaaacttacgagtgccgttgtggactcccagatctcttgctgcaagggagacctctgcaatgcggtggtcctggcagccagcagcccctgggccctgtgcgtacagctcctgctcagcctggggtcagtcttcctctgggccctgctgtgagggcctttcccgccctctcccccgcgggcctaccctctgtccctgtgcgtcaccagctgcttggtttgaagagctg

SEQ ID NO: 18 - protein, 134 aa, Homo sapiens; identical to SEQ ID NO: 2

SEQ ID NO: 19 - DNA, 510 bp, Homo sapiens
  misc_feature (462)..(462): n is a, c, g, or t
ctgaagtttgtctgtgcactggcaccctggaatgagcagtctccaggccatgaagaccttgtccctggtcctgctggtggccctgctgagcatggagagagggtctgcgctgctacagatgcttggcggtcttggaaggggcctcctgcagcgtggtctcgtgccccttcctggatggggtctgtgtctcccagaaagtgagcgtctttggcagtaaagtgagaggggagaacaagctctccctcctctcctgccagaaggacgtcggattccccctgctgaaacttacaagtgccgttgtggactcccagatctcttgctgcaagggagacctccgcaatgcggtggtcctggcagccggcagcccctgggccctgtgcgtacagctcctgctcagcctggggtcagtcttcctctgggccctgctgtgagggcctttcccgccctctcccccgcaggcantaccctctgtccctgtgcgtcaccagctgcttggttttgaagagctgc

SEQ ID NO: 20 - protein, 61 aa, Homo sapiens
MSSLQAMKTLSLVLLVALLSMERGSALLQMLGGLGRGLLQRGLVPLPGWGLCLPESERLWQ

SEQ ID NO: 21 - DNA, 486 bp, Homo sapiens
tgaagtttgtctgtgcactggcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctagatggggtctgtgtctcccagaaagtaaagtgagaggggagaacaagctctccctcctctcctgccagaaggacgtcggattccccctgctgaaacttacaagtgccgttgtggactcccaggtctcttgctgcaagggagacctctgcaatgcggtggtcctggcagccggcagcccctgggccctgtgcgtacagctcctgctcagcctggggtcagtcttcctctgggccctgctgtgagggcctttcccgcccactcccccgcaggcctaccctctgtccctgtgcgtcaccagctgcttggttt

SEQ ID NO: 22 - protein, 59 aa, Homo sapiens; identical to SEQ ID NO: 10

SEQ ID NO: 23 - DNA, 580 bp, Homo sapiens
ctgaagtttgtctgtgcactagcaccctggaatgagcagtctccaggccatgaagaccttgtccctggtcctgctggtggccctgctgagcatggagagaggtgagaagcagaggggcctttagaggactttgttccagcgcactcctgctgccccgtgtgtgctggaactagttgcaggtgggtgtgctcggaaggcgtgcctgctgggggtggcgggcttcggtgttccggtggcagaggtgactggtgtgtttggtgcctgctctgtgcttgttaccgcgcgtgctggctgtgctcacttccgaggactcactgagtcctgggcacgtgtatgcctttggcattgggcagtggctgctggtgcctctggacaaagaggtggtgttggagggttgcaggccaccagttgcaggctgccagttgcaggcaggtgtgtggggctattgcaaaggtccaggtggcaggttgggacaagggtggtggtgagagtgggtgcccttgtgggcatgggactctcaccagggcattggtgtatgtcctggcatgtgcgtcaccagctgcttggtttgaagagctgc

SEQ ID NO: 24 - protein, 30 aa, Homo sapiens
MSSLQAMKTLSLVLLVALLSMERGEKQRGL

SEQ ID NO: 25 - DNA, 497 bp, Homo sapiens
ctgaagtttgtctgtgcactggcaccctggaatgagcagtctccaggccatgaagaccttgtccctggtcctgctggtggccctgctgagcatggagagggctcagggtctgcgctgctacagatgcttggcggtcttggaaggggcctcctgcagcgtggtctcgtgccccttcctggatggggtctgtgtctcccagaaagtaaagtgagaggggagaacaagctctccctcctctcctgccagaaggacgtcggattccccctgctgaaacttacaagtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccagctgcttggtttgaagagctgc

SEQ ID NO: 26 - protein, 59 aa, Homo sapiens; identical to SEQ ID NO: 10

SEQ ID NO: 27 - DNA, 624 bp, Homo sapiens
atgctggggctgctggtggcgttgctggccctggggctcgctgtctttgcgctgctggacgtctggtacctggtgcgccttccgtgcgccgtgctgcgcgcgcgcctgctgcagccgcgcgtccgtgacctgctagctgagcagcgcttcccgggccgcgtgctgccctcggacttggacctgctgctgcacatgaacaacgcgcgctacctgcgcgaggccgactttgcgcgcgtcgcgcacctgacccgctgcggggtgctcggggcgctgagggagttgcgggcgcacacggtgctggcggcctcgtgcgcgcgccaccgccgctcgctgcgcctgctggagcccttcgaggtgcgcacccgcctgctgggctgggacgaccgcgcgttctacctggaggcgcgctttgtcagcctgcgggacggcttcgtgtgcgcgctgctgcgcttccggcagcacctgctgggcacctcacccgagcgcgtcgtgcagcacctgtgccagcgcagggtggagccccctgagctgcccgctgatctgcagcactggatctcctacaacgaggccagcagccagctgctccgcatggagagtgggctcagtgatgtcaccaaggaccag

SEQ ID NO: 28 - protein, 208 aa, Homo sapiens
MLGLLVALLALGLAVFALLDVWYLVRLPCAVLRARLLQPRVRDLLAEQRFPGRVLPSDLDLLLHMNNARYLREADFARVAHLTRCGVLGALRELRAHTVLAASCARHRRSLRLLEPFEVRTRLLGWDDRAFYLEARFVSLRDGFVCALLRFRQHLLGTSPERVVQHLCQRRVEPPELPADLQHWISYNEASSQLLRMESGLSDVTKDQ

SEQ ID NO: 29 - DNA, 375 bp, Homo sapiens
atgcgggggacgcggctggcgctcctggcgctggtgctggctgcctgcggagagctggcgccggccctgcgctgctacgtctgtccggagcccacaggagtgtcggactgtgtcaccatcgccacctgcaccaccaacgaaaccatgtgcaagaccacactctactcccgggagatagtgtaccccttccagggggactccacggtgaccaagtcctgtgccagcaagtgtaagccctcggatgtggatggcatcggccagaccctgcccgtgtcctgctgcaatactgagctgtgcaatgtagacggggcgcccgctctgaacagcctccactgcggggccctcacgctcctcccactcttgagcctccgactg

SEQ ID NO: 30 - protein, 125 aa, Homo sapiens
MRGTRLALLALVLAACGELAPALRCYVCPEPTGVSDCVTIATCTTNETMCKTTLYSREIVYPFQGDSTVTKSCASKCKPSDVDGIGQTLPVSCCNTELCNVDGAPALNSLHCGALTLLPLLSLRL

SEQ ID NO: 31 - DNA, 360 bp, Homo sapiens
atgaggcttctcctggtgctgctgcttctggctgctgtgtgtgctgccctggctcaggccctgcactgccacgtgtgctgcggccatgagcactgcgagtccctggtggagtgtgcccccactgacaaatactgtgtgatcacacgggccaccagccccggtggcatcctggtcatgaagtcctgctccccgacgtgccccaacagcactgtgtcctccgacagccgcgccctctctgtgtcctgctgccagggtagccagtgcaaccgcagtgcagccgcaggcctggtgggcagccccgggaccctgtgggccagcatcgcagccagccagctgtgggccctgctgcaggcagcccgc

SEQ ID NO: 32 - protein, 120 aa, Homo sapiens
MRLLLVLLLLAAVCAALAQALHCHVCCGHEHCESLVECAPTDKYCVITRATSPGGILVMKSCSPTCPNSTVSSDSRALSVSCCQGSQCNRSAAAGLVGSPGTLWASIAASQLWALLQAAR

SEQ ID NO: 33 - DNA, 453 bp, Homo sapiens
atgaaggtcactagccccatgctgctgctggctgagggccagggccttgagtgcttccagtgctacggtgtcctggaccccagcctgtgtcaccccgtctcctatcccatgcaggctcaaagctgcccctcctctgtggtcactggcactatcgatggtgagtcctgggtgggacccagcgtctgtaggcagggcagaagctcagctacactagcaagggctgtggccccactctgtgcccagattatgaacctcacccatcctgtggtccctggagggtcttaccccacagaaattgaggatagactgattgactcgaagattgagaagctggacatgacctgctgtgaaaatagcctccgtaacaaggcggccacagtgcggcgtggcctctggtgccaggctgtcagggagctcctgctcagcctgagccccttcctctgggctctgctg

SEQ ID NO: 34 - protein, 151 aa, Homo sapiens
MKVTSPMLLLAEGQGLECFQCYGVLDPSLCHPVSYPMQAQSCPSSVVTGTIDGESWVGPSVCRQGRSSATLARAVAPLCAQIMNLTHPVVPGGSYPTEIEDRLIDSKIEKLDMTCCENSLRNKAATVRRGLWCQAVRELLLSLSPFLWALL

SEQ ID NO: 35 - DNA, 1030 bp, Homo sapiens
atggagaggctcgtcctaaccctgtgcaccctcccgctggctgtggcgtctgctggctgcgccacgacgccagctcgcaacctgagctgctaccagtgcttcaaggtcagcagctggacggagtgcccgcccacctggtgcagcccgctggaccaagtctgcatctccaacgaggtggtcgtctcttttaggtcagaacaagtgacagaggtcaccaggggctgcaccaacaaccgcatcgtctcggcccgtcccggctgggaggagttcacctgggacagcatcctctgtgccagcgtcttgtgctgtttggagaccctgggtaaccgggaagccatggcaggcagcgctgcccaggccctgcaagggggctgcagctcacccagtggagggcgtcccctgacaaagccgcccctctgtgctgtgaggtgggaggagcctctgcctgtctaccggccccagattccacgcccatcggggaagcccggcaaaggcaccagcactgggaatgtgccccagcaaacagtgagcaacgaggaggctgacggtagtgaggtcacggcacgcaccttgctgatgaccggggttcagccagacgtaactctgggaaaacagactgagctcagccctttcagccagcggccattcagcttttctgaccatggcccatatcaacaaacacaatatggtacccactcttgctggcaccaggacactgagacggtccaggaaacacggcaagcatacgtgtgcaccacactgctcgtcccgtccagctgtggccatgctgaaaattgcaatgggccactggaagacaggttattcaggcaggacacccagaggagcttccagcccgcagtttcagtggtgcccagcaaacagctcctagtggcctctgagggccttgccagcgtggacagctccttgccagtgggacggttccttgccagtgtggacggctcctgctgcagctcccaaagggcgctctcctgccagccttggctgaggcgctgctgcttcagtagggtcc

SEQ ID NO: 36 - protein, 343 aa, Homo sapiens
MERLVLTLCTLPLAVASAGCATTPARNLSCYQCFKVSSWTECPPTWCSPLDQVCISNEVVVSFRSEQVTEVTRGCTNNRIVSARPGWEEFTWDSILCASVLCCLETLGNREAMAGSAAQALQGGCSSPSGGRPLTKPPLCAVRWEEPLPVYRPQIPRPSGKPGKGTSTGNVPQQTVSNEEADGSEVTARTLLMTGVQPDVTLGKQTELSPFSQRPFSFSDHGPYQQTQYGTHSCWHQDTETVQETRQAYVCTTLLVPSSCGHAENCNGPLEDRLFRQDTQRSFQPAVSVVPSKQLLVASEGLASVDSSLPVGRFLASVDGSCCSSQRALSCQPWLRRCCFSRV

SEQ ID NO: 37 - DNA, 423 bp, Homo sapiens
atgaaggcgctcggggctgtcctgcttgccctcttgctgtgcgggcggccagtgctgctgcggtgctacacctgcaagtccctgcccagggacgagcgctgcaacctgacgcagaactgctcacatggccagacctgcacaaccctcattgcccacgggaacaccgagtcaggcctcctgaccacccactccacgtggtgcacagacagctgccagcccatcaccaagacggtggaggggacccaggtgaccatgacctgctgccagtccagcctgtgcaatgtcccaccctggcaaagctcccgagtccaggacccaacaggcaagggggcaggcggcccccggggcagctccgaaactgtgggcgcagccctcctgctcaacctccttgccggccttggagcaatgggggccaggagaccc

SEQ ID NO: 38 - protein, 141 aa, Homo sapiens
MKALGAVLLALLLCGRPVLLRCYTCKSLPRDERCNLTQNCSHGQTCTTLIAHGNTESGLLTTHSTWCTDSCQPITKTVEGTQVTMTCCQSSLCNVPPWQSSRVQDPTGKGAGGPRGSSETVGAALLLNLLAGLGAMGARRP

SEQ ID NO: 39 - protein, 348 aa, Homo sapiens
MKTLSLVLLVALLSMERAQGLRCYRCLAVLEGASCSVVSCPFLDGVCVSQKVSVFGSKVRGENKLSLLSCQKDVGFPLLKLTSAVVDSQISCCKGDLCNAVVLAASENLYFQGASQEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 368 aa, Homo sapiens
MGSLQAMKTLSLVLLVALLSMERAQGLRCYRCLAVLEGASCSVVSCPFLDGVCVSQKVSVFGSKVRGENKLSLLSCQKDVGFPLLKLTSAVVDSQISCCKGDLCNAVVLAASSPWALCVQLLLSLGENLYFQGASQEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 3907 bp, Homo sapiens
tgagtaaattggcctggagtcacctattgctccagtagcaagttcctgtgaaatggaaattcaggaacccacccttgttctgggccagtcaggaagagcctgaggggaggagccgctgagggcagggcctctccctgccccaccttcctctctctggcaagcctgcagggcaagtggctgagaatcagccagagggaggaaagtgaggaggggtggaccaggcctgggagggtcctgggtgggggcgctaaggagaggaggtgcagtacagccatggggctctgggtgagaattgcagggaaggatgggagggaggaatggggactggggtgggggcgtgaggcagacagttctcctttgggtgactgtgctgaggacagggtgggagatggagagccggatgttctggctttgctgctgtgcaggactcattcgatccccaggagggtgaggagtccgagggctgctctgattcgctgaagtttgtctgtgcactagcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtgagcgtctttggcagtaaagtgagaggggagaacaagctctccctcctctcctgccagaaggacgtcggattccccctgctgaaacttacaagtgccgttgtggactcccagatctcttgctgcaagggagacctctgcaatgcggtggtcctggcagccagcagcccctgggccctgtgcgtacagctcctgctcagcctggggtcagtcttcctctgggccctgctgtgagggcctttcccgccctctcccccgcaggcctaccctctgtccctgtgcgtcaccagctgcttggttttgaagagctgcctcactgaattgcaggaattctggtgtggcttttcagccatgagatcaggggaaggatgtggccagggcagaggtcggggcttccctgtcagggaacatatccctgcttaaactccttgtcctctgggcccaggccctccagcccatgccccccaacaacaccacttttttcagagctgggcctcagagaccaggccccaccactggctgcttgggacctagggtcatagagaaaccgtggcccagggcgggagtccccagctccatccagcttgccactaacttgccgaggagccagggccaacccctcagctctctgggctacctgaatccaagcaaggagggaacgtggtcccagggggccctgctggcctcccacacaggatgagggcctccctctcaaggtcaacaaacagccctttgctgacccactcagcgtcagcactcggcttcttcctcaccattcctgaggccgcccacctgccactctgacctcaggcctctgcaacatcctgctggcaccatggccttggggaggtgctcctggggcacgggtgcccccccagggaccagaacccacagagtgccaggctcatggctcttctggcagaactcttgccatgtggctccagggaacatctgagtagggttgtcagagagcctctggatggtcaccaagaggcagagctcctgcggccaccagccatcctgtggtgtgggccaggcctgtcatttctgtgccctggctagggctcccatgatgccccgtgtcaggggttagactggtctcccctggtggtgggagaaaggggctccttcaggactgcactctggacagaaagtcagggctgggctcagaatggtctgggtggacggtcatctcagcctggagtcagggctgggctgagaacgccctgggtggacggtcgtctcagcctggagtcagggctgggctgagaacgccctgggtggacggccgtctcagcctggagtcagggctgggctgagaacgccctgggtggacggccgtctcagcctggagtcagggctgggctgagaacgccctgggtggacggccgtctcagcctggagtcagggctgggctgagaacgccctgggtggacggccgtctcagcctggagtcagggctgggctgagaacgccctgggtggacggccgtctcagcctggagtcagggctgggctgagaacgccctgggtggacggccgtctcagcctggagtcagggctgggctgagaacgccctgggtggacggccgtctcagcctggagtcagggctgggctcagaacagtctgggtggacggctattgtcagggaccccaaacagagggaccggctgaagccatggaagaagaacataaattgtgaagatttcatggacatttattagttccccaaattaatacttttataatttcttatgcctgtctttactgcaatctctgaacataaattgtgaagatttcatggacacttatcacttccccaatcaatacccttgtgatttcctatgcctgtctttaatcttttaatcccatcatcttcgtaagctgaggaggatgtatattgcctcaggaccctgtgatgattgtgttaactgcacaaattgtttgcagagcatgtgtgtttgaacaatatgaaatctgggtaccttgaaaaaagaacagggtaacagcaacgttcagggaacaagagagatcaccttaaactctggttgcctgtgagccaggtgaaacagagccatatttctcttctttcaaaagcaaataggagaaatatcgctgaattctttttctcagcaaggaacatccctgagaaagagaatgtatccctaaggggaggcctctgaaatgtctgctttggggacggctgccttttacagttgtagataagggatgaaataagccccagtctcccgcagtgctcccaggcttattaggatgaggaaattcccgcctaataaattttggtcagactggttgtctgctctcaaaccctgtctcctgataagatgttatcaatgacaatgtgtgcccaaaacttcattagcaattttaatttcgccccggtcccgtggtcctgtgatctcgccctgcctccgtttgccttgtgatatcttattaccttgtgaagcatgtgatctctgtgacccacatcctatttgtacactccctccccttctgaaaatcactaataaaaatttgctggttttacggctcagggggcatcacggaacctgccgacatgtgatgtctcccccggacgcccagctttgaaatttctctcttttgtactctgtccctttatttctcagactggccgacacttagggaaaatagaaaagaacctacatgaaatatcgggggtgaatttcccccgatagatgaccatctcagcctggggtcagggcagggctcagaatggtctgggtggacggccgtctcagcctggagtcagggcagggctcaaggtctgggtgaatggccatctcagcctggagtcagggctgggctcagacgaccagcagtctttgacctgccatgccgggagccacatctcgtgaccaggtcctggccttccacgctggccagacgcctgcatccaagacgtcctggtccctctgactgcacagtgtgacagccaccacccagccatgccctgtactcctggatgtgatttgctaattagggtcaatggggtaagatgcccaattaaatatgaatttcagataaacaatttttagcataagtatgtcccaaacattggtaaaaatgactatttgttgtttatctgagattcagatcaaactgggcatcttggacattttatttgctcaactagaaattcatgagtgcctaaggagctcaagggacattccagaccccccaaatctgt

SEQ ID NO: 42 - DNA, 25 bp, Artificial sequence
  Synthetic PCR Primer
catgaagaccttgtccctggtcctg

SEQ ID NO: 43 - DNA, 20 bp, Artificial sequence
  Synthetic PCR Primer
gcccagaggaagactgaccc

SEQ ID NO: 44 - DNA, 18 bp, Artificial sequence
  Synthetic PCR Primer
ggctgctggtggcgttgc

SEQ ID NO: 45 - DNA, 24 bp, Artificial sequence
  Synthetic PCR Primer
ctcgttgtaggagatccagtgctg

SEQ ID NO: 46 - DNA, 22 bp, Artificial sequence
  Synthetic PCR Primer
gacatcactgagcccactctcc

SEQ ID NO: 47 - DNA, 20 bp, Artificial sequence
  Synthetic PCR Primer
tgctggctgcctgcggagag

SEQ ID NO: 48 - DNA, 21 bp, Artificial sequence
  Synthetic PCR Primer
caagagtgggaggagcgtgag

SEQ ID NO: 49 - DNA, 21 bp, Artificial sequence
  Synthetic PCR Primer
cagtcggaggctcaagagtgg

SEQ ID NO: 50 - DNA, 22 bp, Artificial sequence
  Synthetic PCR Primer
cttctcctggtgctgctgcttc

SEQ ID NO: 51 - DNA, 22 bp, Artificial sequence
  Synthetic PCR Primer
cttctggctgctgtgtgtgctg

SEQ ID NO: 52 - DNA, 20 bp, Artificial sequence
  Synthetic PCR Primer
gctggctggctgcgatgctg

SEQ ID NO: 53 - DNA, 23 bp, Artificial sequence
  Synthetic PCR Primer
gaaggtcactagccccatgctgc

SEQ ID NO: 54 - DNA, 20 bp, Artificial sequence
  Synthetic PCR Primer
ccatgctgctgctggctgag

SEQ ID NO: 55 - DNA, 25 bp, Artificial sequence
  Synthetic PCR Primer
ggctattttcacagcaggtcatgtc

SEQ ID NO: 56 - DNA, 22 bp, Artificial sequence
  Synthetic PCR Primer
gaggctcgtcctaaccctgtgc

SEQ ID NO: 57 - DNA, 20 bp, Artificial sequence
  Synthetic BIG6-41f
ctgtggcgtctgctggctgc

SEQ ID NO: 58 - DNA, 21 bp, Artificial sequence
  Synthetic PCR Primer
ccgtccacactggcaaggaac

SEQ ID NO: 59 - DNA, 20 bp, Artificial sequence
  Synthetic PCR Primer
cttgccctcttgctgtgcgg

SEQ ID NO: 60 - DNA, 20 bp, Artificial sequence
  Synthetic PCR Primer
cccacagtttcggagctgcc

SEQ ID NO: 61 - DNA, 20 bp, Artificial sequence
  Synthetic PCR Primer
cggcaaggaggttgagcagg

SEQ ID NO: 62 - protein, 134 aa, Mus musculus
MDTSHTTKSCLLILLVALLCAERAQGLECYQCYGVPFETSCPSITCPYPDGVCVTQEAAVIVDSQTRKVKNNLCLPICPPNIESMEILGTKVNVKTSCCQEDLCNVAVPNGGSTWTMAGVLLFSLSSVLLQTLL

SEQ ID NO: 63 - protein, 134 aa, Homo sapiens; identical to SEQ ID NO: 2

SEQ ID NO: 64 - protein, 167 aa, Homo sapiens
SQARNDBCQEZGHSQILKMFPSTWYVSQTHERSMSSLQAMKTLSLVLLVALLSMERAQGLRCYRCLAVLEGASCSVVSCPFLDGVCVSQKVSVFGSKVRGENKLSLLSCQKDVGFPLLKLTSAVVDSQISCCKGDLCNAVVLAAGSPRALCVQLLLSLGSVFLWALL

SEQ ID NO: 65 - protein, 169 aa, Homo sapiens
SQARNDBCQEZGHSQILKMFPSTWYVSQTHERSMSSLQAMKTLSLVLLVALLSMERAQGLRCYRCLAVLEGASCSVVSCPFLDGVCVSQKVSLSLSKKRRKEKNKLSLLSCQKDVGFPLLKLTSAVVDSQISCCKGDLCNAVVLAASSPWALCVQLLLSLGSVFLWALL

SEQ ID NO: 66 - protein, 189 aa, Homo sapiens
SQARNDBCQEZGHSQILKMFPSTWYVSQTHERSMGVFHDYSQRVGRGVARKHVLGGSSGCTRAVAAPACSYVACVISILNLHQGCVFLPSLPAQGLRCYRCLAVLEGASCSVVSCPFLDGVCVSQKVSVLAVSPWGARAEGRLSAVVDSQISCCKGDLCNAVVLAAGSPWALCVQLLLSLGSVFLWALL

SEQ ID NO: 67 - protein, 218 aa, Homo sapiens
SQARNDBCQEZGHSQILKMFPSTWYVSQTHERSMGVFHDYSQRVGRGVARKHVLGGSSGCTRAVAAPACSYVACVISILNLHQGCVFLPSLPAQGLRCYRCLAVLEGASCSVVSCPFLDGVCVSQKVSVFGSESLGCQGRGQVKCRCGLPDLLLQGRPLQCGGPGSRQPLGPVRTAPAQPGVSLPLGPAVRAFPALSPAGLPSVPVRHQLLGFEELPH

SEQ ID NO: 68 - protein, 172 aa, Homo sapiens
SQARNDBCQEZGHSQILKMFPSTWYVSQTHERSHQGCVFLPSLPAQGLRCYRCLAVLEGASCSVVSCPFLDGVCVSQKVSVFGSESLGCQGRGQVKCRCGLPDLLLQGRPLQCGGPGSRQPLGPVRTAPAQPGVSLPLGPAVRAFPALSPAGLPSVPVRHQLLGFEELPHLE

SEQ ID NO: 69 - DNA, 44 bp, Artificial sequence
  Synthetic PCR Forward Primer
actagcggatccctcaccatgggcagtctccaggccatgaagac

SEQ ID NO: 70 - DNA, 32 bp, Artificial sequence
  Synthetic PCR Reverse Primer
ctcctggctagcgctggctgccaggaccaccg

SEQ ID NO: 71 - DNA, 35 bp, Artificial sequence
  Synthetic PCR Reverse Primer
ctcctggctagcccccaggctgagcaggagctgta

SEQ ID NO: 72 - DNA, 20 bp, Artificial sequence
  Synthetic GAPDH Forward Primer
accacagtccatgccatcac

SEQ ID NO: 73 - DNA, 20 bp, Artificial sequence
  Synthetic GAPDH Reverse Primer
tccaccaccctgttgctgta

SEQ ID NO: 74 - DNA, 22 bp, Artificial sequence
  Synthetic LY6-BIG1 PCf1 Forward Primer
ctgaagtttgtctgtgcactag

SEQ ID NO: 75 - DNA, 22 bp, Artificial sequence
  Synthetic LY6-BIG1 PCr1 Reverse Primer
gcagctcttcaaaaccaagcag

SEQ ID NO: 76 - DNA, 6 bp, Homo sapiens
aataaa

SEQ ID NO: 77 - DNA, 25 bp, Artificial sequence
  Synthetic Sca-1 PCf2 Primer
catgaagaccttgtccctggtcctg

SEQ ID NO: 78 - DNA, 22 bp, Artificial sequence
  Synthetic Sca-1 PCr1 Primer
ctgcttggttttgaagagctgc

SEQ ID NO: 79 - DNA, 24 bp, Artificial sequence
  Synthetic Sca-1 PCr2 Primer
aggcttattaggatgaggaaattc

SEQ ID NO: 80 - DNA, 26 bp, Artificial sequence
  Synthetic Sca-1 PCf3 Primer
caagagagatcaccttaaactctggt

(19) United States
(12) Patent Application Publication
(10) Pub. No.: US 2012/0064080 A1
(43) Pub. Date: Mar. 15, 2012

(54) Human LY6-Big Molecules and Methods of Use

(75) Inventors: Peter CHU, San Diego, CA (US); Mark Daniels, San Diego, CA (US); Robert Peach, San Diego, CA (US); Karen McLachlan, Encinitas, CA (US); Jaeho Jung, San Diego, CA (US); Scott Glaser, San Diego, CA (US)

(73) Assignee: Biogen Idec MA Inc.

(21) Appl. No.: 13/070,468

(22) Filed: Mar. 23, 2011

Related U.S. Application Data
(63) Continuation of application No. 11/665,986, filed on Nov. 27, 2007, now abandoned.
(60) Provisional application No. 60/620,678, filed on Oct. 22, 2004.

(51) Int. Cl. A61K 39/395; C07K 14/00; A61P 35/00; C07K 16/00; A61P 37/02; C07H 21/04; C12N 5/071

(52) U.S. Cl. 424/139.1; 536/23.1; 530/350; 530/324; 435/325; 530/387.9; 435/375

(57) ABSTRACT

The present invention is directed to human Ly6-BIG molecules and their use in diagnostic, prognostic, and treatment methods for colon, lung and other cancers, in preventing the reoccurrence of such cancers, and in diagnostic, prognostic, and treatment methods for autoimmune disorders and AIDS.

### BACKGROUND OF THE INVENTION

1. Field of the Invention

The present invention is directed to human Ly6-BIG molecules and their use in diagnostic, prognostic, and treatment methods for colon, lung and other cancers, in preventing the reoccurrence of such cancers, and in diagnostic, prognostic, and treatment methods for autoimmune disorders and AIDS.

2. Background Art

Cancer is a significant health problem throughout the world. Although advances have been made in detection and therapy of cancer, no vaccine or other universally successful method for prevention or treatment is currently available. One reason for failure of a cancer treatment is often the growth of secondary metastatic lesions in distant organs. Therapy for metastasis currently relies on a combination of early diagnosis and aggressive treatment, which may include radiotherapy, chemotherapy or hormone therapy. However, the toxicity of such treatments limits the use of presently available anticancer agents for treatment of malignant disease. The high mortality rate for many cancers indicates that improvements are needed in metastasis detection, prevention and treatment.

The development of less toxic antitumor agents would facilitate the long term treatment of latent or residual disease. Such agents could also be used prophylactically after the removal of a precancerous tumor.

In addition, the ability to detect cancer cells that are more primitive (e.g., cancer stem cells) would allow for better methods for detecting cancers at earlier stages, and for better methods of detecting (e.g., prognostic methods) or targeting (e.g., therapeutic methods) those cancer cells that are, or are more likely to become, metastatic.

Accordingly, there is a need in the art for the development of further methods for detecting, inhibiting, and treating cancer, e.g., metastasis.

In addition, cancer therapies such as bone marrow transplantation or peripheral stem cell therapy require the identification and purification of hematopoietic stem cells (HSC). However, current methods do not preferentially identify the most primitive or totipotent stem cells. Thus, a larger population of HSC is required to reconstitute the immune system than would otherwise be required.

Accordingly, there is also a need in the art for methods to detect more primitive or totipotent HSC for bone marrow transplant and peripheral stem cell transplant.

### BRIEF SUMMARY OF THE INVENTION

The present invention is directed to novel human Ly6-BIG (Ly6-BIG1-7) polypeptides, including fragments, fusions, mutants and variants thereof. The present invention also provides splice variants of human Ly6-BIG1. The present invention also provides polynucleotides encoding such polypeptides, and antibodies against such polypeptides. The present invention also provides Ly6-BIG binding molecules (e.g., antisense oligonucleotides, RNAi, siRNA).

The invention further provides methods of isolating hematopoeitic stem cells, tissue stem cells (i.e., normal, non-hematopoietic stem cells), and cancer stem cells, and provides methods of diagnosing, prognosing, and treating cancers (e.g., cancer immunotherapy), and provides methods of diagnosing, prognosing, and treating autoimmune diseases using the human Ly6-BIG polypeptides, polynucleotides, antibodies, and binding molecules of the invention. Additional uses are also described.

### BRIEF DESCRIPTION OF THE DRAWINGS/FIGURES

FIGS. 1A-1B: Design of primers to amplify human Sca-1 cDNA gene sequences. Three sets of primers were designed to detect the predicted hLy6-BIG1 cDNA sequence identified by searching ESTs from the human chromosome 8q24.3 region. Primer pair 1 (Pr1; SEQ ID NOs:______) amplifies the entire open reading frame (512 bp), primer pair 2 (Pr2; SEQ ID NOs:______) amplifies 466 bp segment of the ORF, and primer pair 3 (Pr3; SEQ ID NOs:______) amplifies a 263 bp region in the 3′ untranslated region (UTR).

FIG. 2: Alignment of the Amino Acid Sequences of cloned human Ly6-BIG1 (SEQ ID NO:______) and mouse Sca-1 (SEQ ID NO:______). Underlining indicates 6 amino acids that are added to the N-terminus. Double-underlined indicates that S in the original sequence is modified to G when cloned.

FIG. 3: hLy6-BIG1 is expressed in a human cDNA tissue panel. cDNA from human whole brain, brain temporal cortex, brain cerebral cortex, spleen, colon, small intestine, lung and leukocyte was each found to be positive for hLy6-BIG1 using all primer pairs Pr1, Pr2 and Pr3. Brain occipital cortex and skeletal muscle was faintly positive with Pr1 and Pr2, but positive using Pr3. GAPDH PCR shows that the amount of cDNA in each sample was approximately equivalent.

FIG. 4: hLy6-BIG1 is expressed in bone marrow CD133+ stem cells, and other additional tissues. Expression of hLy6-BIG1 was examined using Pr1. The expected 512 bp band was found in cDNA from liver, pancreas, lung, kidney, brain, and bone marrow CD133+ stem cells.

FIG. 5: hLy6-BIG1 is expressed in several different human stem cell compartments, as well as normal and tumor cells. Using Pr3, hLy6-BIG1 expression was detected by PCR in bone marrow CD34+, bone marrow CD133+ and cord blood CD34+ stem cells. hLy6-BIG1 expression was also seen in normal brain, thymus, testis, prostate, placenta, and ovary. hLy6-BIG1 expression was also seen in colon adenocarcinoma and lung carcinoma.

FIG. 6: hLy6-BIG1 is expressed in tumor cell lines. Using Pr3, hLy6-BIG1 expression was detected by PCR in tumor cell lines HPAF2, Su86.86, and SW620.

FIG. 7: Analysis of cloned hLy6-BIG1 sequences. PCR products amplified by Pr1 from brain, spleen, and small intestine tissue cDNA were cloned into the T Easy pGem vector, and sequenced. 5 out of 10 clones sequenced corresponded to the two predicted hLy6-BIG1 gene sequences. These two different variants are the product of alternate usage of exon 2 and exon 3 due to alternate splicing.

FIGS. 8A-8C: Clustal multiple sequence alignment of the Ly6-BIG1.1-1.13 proteins of the invention (SEQ ID NOs:2, 4, 6, 8, 10, 12, 14, 16, 18, 20, 22, 24, and 26).

FIGS. 9A-9S: Hydropathy plots of the Ly6-BIG proteins of the invention. FIGS. 10A-10G: PepPlots of the Ly6-BIG1.1 (FIG. 10A) and 2-7 (FIGS. 10B-10G) proteins (SEQ ID NOs:2, 28, 30, 32, 34, 36, and 38). PepPlot determination of protein secondary structure and hydrophobicity. (Gribskov and Devereux, Nucl. Acids Res. 14(1); 327-334 (1986)). The GCG Manual, Accelrys Inc. (1982-2002) is herein incorporated by reference. The sequence is shown in the first part of the plot. The residue schematic is shown in the second part of the plot.

The Chou and Fasman beta-Sheet forming and breaking residues are shown in the third panel (Adv. Enz. 47; 45-147 (1978)). The Chou and Fasman alpha and beta propensities are shown in the fourth panel. The Chou and Fasman alpha-helix forming and breaking residues are shown in the fifth panel (1978 cited above). The Chou and Fasman amino ends are shown in the sixth panel. The Chou and Fasman carboxyl ends are shown in the seventh panel. The Chou and Fasman turns are shown in the eighth panel. The hydrophobic moment at each position of the sequence is shown in the ninth panel. (calculation as in Eisenberg et al. (Proc. Natl. Acad. Sci. USA 81; 140-144 (1984)), except that the hydrophobic moment has been normalized in the window where the moment is being determined, as in the method described by Finer-Moore and Stroud (Proc. Natl. Acad. Sci. USA, 81; 155-159 (1984)). The Kyte and Doolittle hydropathy is indicated. The Kyte and Doolittle hydropathy measure (J. Mol. Biol. 157; 105-132 (1982)) and the Goldman, Engelman, and Steitz (GES) curve, showing transbilayer helices. (reviewed in Ann. Rev. Biophys. Biophys. Chem. 15; 321-353 (1986)) are also indicated. For both curves, hydrophobic regions are in the upper half of the frame, while hydrophilic regions are in the lower half.

FIG. 11: Expression of Ly6-BIG1 Fc fusion protein.

FIG. 12: Alignment of Ly6-BIG2-6 (SEQ ID NOs:28, 30, 32, 34, and 36) and NOV8a-8e (SEQ ID NOs:______).

FIG. 13: Summary of FACS and western blot analysis of anti-Ly6-BIG1 monoclonal staining of BIG1-CHO transfectants. The ability of anti-BIG1 mABs to specifically bind to CHO cells expressing cell surface BIG1 protein, or control CHO cells not expressing BIG1 protein, was tested by flow cytometry (FACs) analysis. All six antibodies listed were positive for specific staining of CHO-BIG1 transfectants. Furthermore, 26G6 and 29F6 mAbs also were shown to specifically bind to BIG1 protein in lysates from CHO-BIG1 cells by western analysis. FACS=Fluorescence activated cell sorting. MFI=Mean fluorescence intensity. MFIR=Mean fluorescence intensity ratio (ratio of mAb staining to experimental sample to the level of mAb staining to a negative control sample). An MFIR of 1 means that the mAb being tested does not specifically stain the experimental sample.

FIG. 14: Western analysis of CHO-BIG1 cells with anti-Ly6-BIG1 monoclonal antibodies 26G6 and 29F6. One million CHO (−lanes) or CHO-BIG1 (+lanes) cells were each pelleted, resuspended in m-PER buffer (Pierce), boiled for 10 minutes, then cleared by centrifugation for 10 minutes at 14,000 rpm. Cleared supernatant representing 1 million cell equivalents was then loaded into a 10% Nu-PAGE gel, electrophoresed at 200V for 1 hour, transferred at 100V onto PDVF membrane, blocked with blocking buffer (5% non-fat dry milk in PBS) for 1 hour, probed with either 29F6 or 26G6 primary antibody in blocking buffer for 1 hour, washed 5 times with PBS+0.05% Tween-20, then probed in blocking buffer with 1:5000 dilution of goat-anti-mouse IgG heavy and light chain secondary antibody-conjugated to horseradish peroxidase (HRP). HRP was detected using KPL ECL kit according to manufacturer's instructions, exposed for 30 minutes on Kodak film, followed by film development in an x-ray developer. Band represents expected ˜10 kDa BIG-1 protein detected by anti-BIG1 monoclonal antibodies 26G6 and 29F6 in CHO-BIG1 (+) lanes, and no band detected in CHO control (−) lanes, as labelled.

### DETAILED DESCRIPTION OF THE INVENTION

The murine Ly-6 protein family is a family of cell surface glycoproteins with distinct subfamilies having an interesting overlapping pattern of tissue expression. Some members are expressed during hematopoiesis from multipotential stem cells to lineage committed precursor cells, while some are expressed on specific leucocyte subpopulations and some are expressed in non-lymphoid tissues. In vitro studies indicate that Ly-6 proteins play a role in T cell activation (Gumley et al., Immunol Cell Biol. 73(4):277-96 (1995)); in the regulation of hematopoietic stem cell development, in the regulation of the development of committed progenitors, megakaryocytes and platelets (Ito et al., Blood 101:517-523 (2003)); and in tumor progression (Eshel et al., Cancer Biology 12:139-147 (2002) and Witz, J. Cell. Biochem. Suppl. 34:61-66 (2000)).

Murine Sca-1/Ly-6A is a glycosyl phosphatidylinositol (GPI)-linked cell surface molecule that is routinely used as a standard marker for identifying and isolating mouse hematopoietic stem cells. (Gumley, et al., Immunol. and Cell Biol. 73:277-96 (1995)). Sca-1/Ly-6A has also been established as a useful marker to identify stem cells in mammary gland epithelial cells, indicating that this protein is also expressed in non-hematopoietic tissues. (Welm, et al., Devel. Biol. 245, 42-56 (2002)). Finally, mouse tumor cells with high Ly6-A/E expression were found to have higher tumorigenicity and significantly higher metastasis in vivo, compared to tumor variants with low Ly-6A/E expression. (Katz, et al., hit. J. Cancer 59(5):684-91 (1994); Eshel R, et al., Semin. Cancer Biol. 12(2):139-47 (2002)). Isolation of normal and cancer stem cells will allow for the development of new treatments for a myriad of human diseases and conditions including cancer, autoimmunity, neurological disorders, bone disease and regenerative medicine. Currently, there are very few established stem cell markers for isolating non-hematopoietic human stem cells. In additional, a novel gene such as human Ly6-BIGA that is upregulated on highly malignant and metastatic tumors may be a new target for cancer therapy. hLy6-BIG1 and its related family members may be useful for the development of new therapies for human disease.

WO 02/018518, published Oct. 17, 2002 (application no. PCT/US02/05374, filed Feb. 21, 2002) discloses several nucleic acid and protein sequences, designated therein as NOV8a-8f, with partial identity to various mouse, rat and human Ly6 molecules.

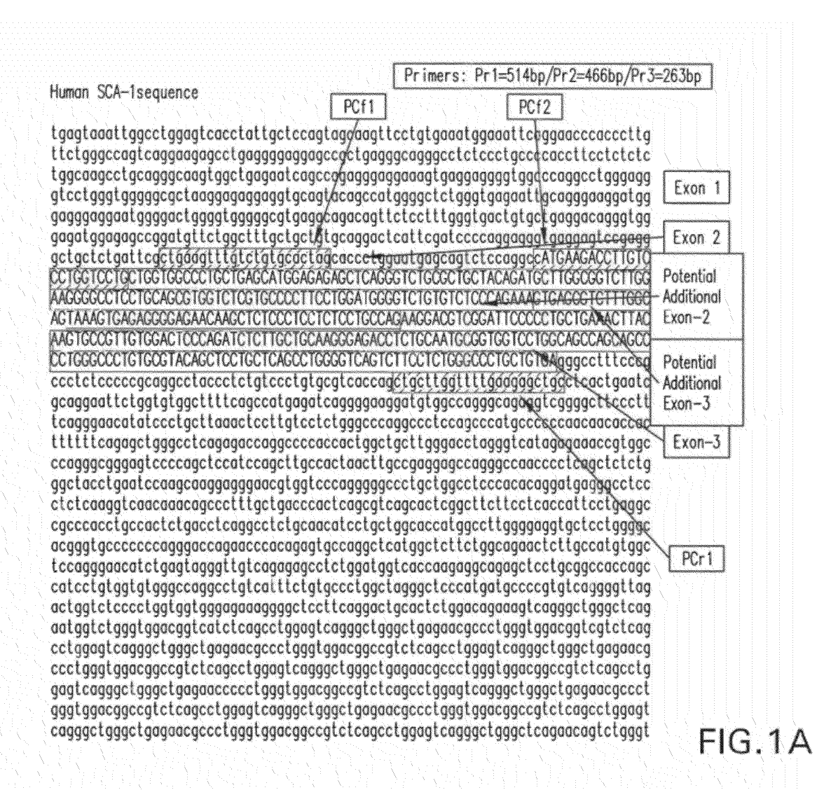

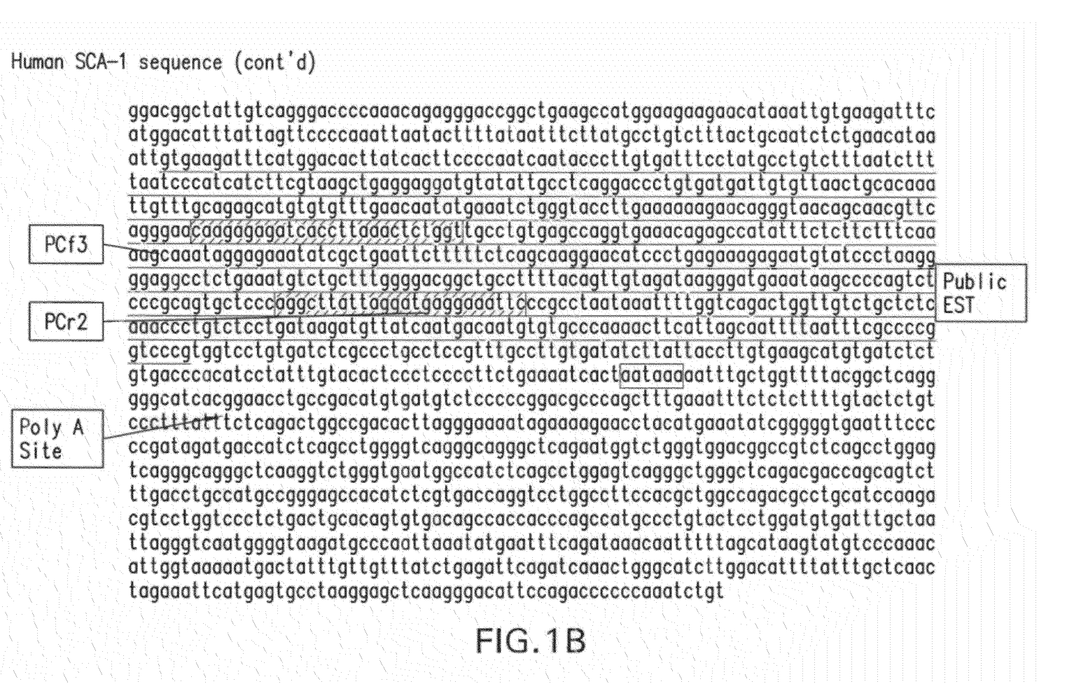

The present inventors have discovered a family of novel Ly6 genes. Seven novel human Ly6 genes, designated human Ly6-BIG1-7 (hLy6-BIG1-7) were identified (Tables 1-19 and FIG. 1) The polypeptides, genes, polynucleotides and antibodies corresponding to hLy6-BIG1-7 are collectively referred to herein as “Ly6-BIG” polypeptides (proteins)/genes/polynucleotides (nucleic acids)/antibodies/binding molecules of the invention, or “human Ly6-BIG” polypeptides (proteins)/genes/polynucleotides (nucleic acids)/antibodies/binding molecules or “hLy6-BIG” polypeptides (proteins)/genes/polynucleotides (nucleic acids)/antibodies/binding molecules or “hLy6-BIG molecules.”

The present inventors have generated a polynucleotide clone encoding the Ly6-BIG1 polypeptide, and hybridoma cell lines that express antibodies that immunospecifically bind the Ly6-BIG1 polypeptide. Thus, the invention encompasses this clone and these cell lines, all of which were deposited with the American Type Culture Collection (“ATCC”) as listed below and given the ATCC Deposit Numbers identified below. The ATCC is located at 10801 University Boulevard, Manassas, Va. 20110-2209, USA. The ATCC deposits were made pursuant to the terms of the Budapest Treaty on the international recognition of the deposit of microorganisms for purposes of patent procedure.

| Identification Reference | Deposit Date | Deposit Number |
|---|---|---|
| Hybridoma 7F6-E12-A5 | Feb. 25, 2005 | PTA-6611 |
| Hybridoma 31A3-D4-H4 | Feb. 25, 2005 | PTA-6612 |
| Hybridoma 26G6-C1-F1 | Oct. 21, 2005 | |
| Hybridoma 29F6-E7-G4 | Oct. 21, 2005 | |
| Hybridoma 5B8-1A9-E10 | Oct. 21, 2005 | |
| Ly-BIG1 Clone | | |

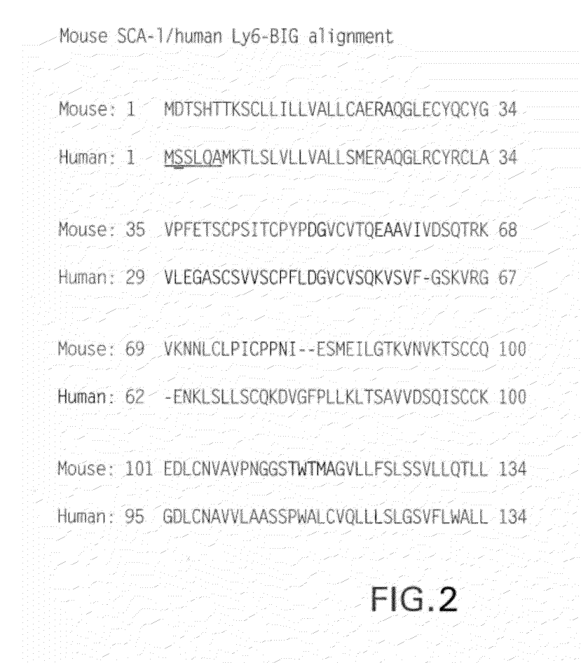

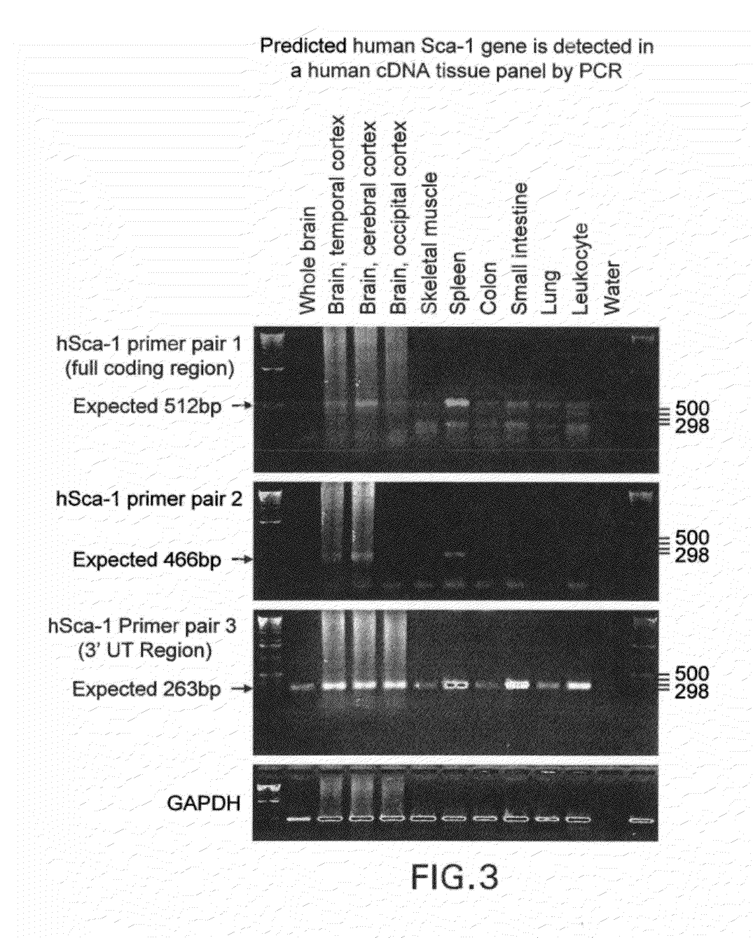

One of these novel hLy6-BIG genes, hLy6-BIG1, is the human homolog to mouse Ly6-A/Sca-1, having 42% amino acid identity, and 57% protein sequence similarity (See, e.g., Tables 1-19, FIGS. 1 and 2, and Example 1). The presence of hLy6-BIG1 was confirmed by RT-PCR analysis to be expressed in a wide variety of human tissues of hematopoietic and epithelial origin (FIGS. 3, 4, 5 and 6).

Sequencing of the complete coding region of hLy6-BIG1 amplified by RT-PCR identified at least 13 versions of the hLy6-BIG1 transcript, due to alternate splicing. The 13 splice variants are collectively referred to herein as “Ly6-BIG1” polypeptides (proteins)/genes/polynucleotides (nucleic acids)/antibodies/binding molecules of the invention, or “human Ly6-BIG1” polypeptides (proteins)/genes/polynucleotides (nucleic acids)/antibodies/binding molecules or “hLy6-BIG1” polypeptides (proteins)/genes/polynucleotides (nucleic acids)/antibodies/binding molecules or “hLy6-BIG1 molecules.” These variant hLy6-BIG1 transcripts and the polypeptides they encode are specific to different cell types, for example, version 1 is specific to stem cells, whereas version 2 is specific to differentiated progenitor cells. Expression of different hLy6-BIG1 versions on different tumors also correlates with increased or decreased malignancy and/or metastatic potential.

The Ly6 domains of the Ly6-BIG proteins of the invention are shown in Table 22. Ly6-BIG proteins of the invention also may include the native signal sequences (leader sequences), which can be identified by known methods. For example, Ly6-BIG 1.1, 1.2, 1.3, 1.5, 1.8, 1.9, 1.11, and 1.13 contain a signal sequence at about amino acids 1-26. Polypeptides of the invention (e.g., fusion proteins such a Fc fusions) may contain the native (Ly6BIG) signal sequence, or may contain no signal sequence, or may contain a replacement of the native signal sequence with a heterologous signal sequence.

The Ly6-BIG1 variants (Ly6-BIG1.1-1.13) and other Ly6-BIG molecules (Ly6-BIG2-7) allow one to distinguish between an epithelial stem cell and its more differentiated progenitors or mature progeny. hLy6-BIG1 variants may also be used to generate antibodies specific to non-stem cells, which could be used in a negative selection process to produce an enriched, or preferably, a purified stem cell population that can be used to obtain stem cell specific targets. Additionally, the expression of hLy6-BIG1 variants may be used to identify various stages of T and B cell development and/or activation, and to isolate these cells for development of immunotherapies or drugs to treat cancer, AIDS and autoimmune disorders.

Another use for hLy6-BIG (e.g., hLy6-BIG1) is for the isolation of hematopoietic stem cells (HSC), for bone marrow transplantation and for gene therapy to treat genetic diseases and cancer. Based on the ability of anti-mouse Sca-1 antibodies to highly enrich (1000-fold) for stem cells that can completely reconstitute hematopoiesis in mice, hLy6-BIG (e.g., hLy6-BIG1) antibodies, alone or in combination with other antibodies (e.g., CD34 and/or CD133 antibodies), may be used to identify a human HSC that is at least as primitive or totipotent, or even more primitive or totipotent, as HSC selected using current protocols (i.e. CD34+ and/or CD133+ isolation). This would allow for bone marrow transplantation and engraftment of human patients possibly with fewer and more pure HSCs than is currently possible.

hLy6-BIG (e.g., hLy6-BIG1) antibodies, or a ligand to hLy6-BIG (e.g., hLy6-BIG1), may be used to activate or inhibit the signaling activity of hLy6-BIG (e.g., hLy6-BIG1). Studies have shown that Sca-1 has a functional role in regulating the development of HSCs and progenitor cell populations. (Ito C Y, et al., Blood 101(2):517-23 (2003)). hLy6-BIG (e.g., hLy6-BIG1) antibodies, Ly6-BIG polypeptides (e.g., Fc fusions), or a hLy6-BIG (e.g., hLy6-BIG1) ligand may be used to activate hematopoietic or non-hematopoietic stem cells to divide, either symmetrically or asymmetrically, yielding a method to expand identical progeny stem cells from existing stem cells. This method to activate stem cells to divide either symmetrically or asymmetrically could also be achieved using antibodies, polypeptides (e.g., Fc fusions), and/or ligands to the other human Ly6 family members disclosed herein.

A differentiated progenitor cell expressing hLy6-BIG (e.g., hLy6-BIG1) may also be activated with hLy6-BIG (e.g., hLy6-BIG1) antibodies, Ly6-BIG polypeptides (e.g., Fc fusions), or a hLy6-BIG (e.g., hLy6-BIG1) ligand, causing it to de-differentiate, and return to functional stem cell status.

Murine Sca-1 is expressed on activated T cells, and hLy6-BIG (e.g., hLy6-BIG1) antibodies may be used to modulate activation of T cells and other cells. Modulation of lymphocytes may be useful for generating cancer immunotherapies or treatments for autoimmune disorders.

Sca-1 is required for self-renewal of mesenchymal progenitors involved in bone formation in mice, and hLy6-BIG (e.g., hLy6-BIG1) may be used to develop therapies for bone disease such as osteoporosis.

Signaling through cell surface hLy6-BIG (e.g., hLy6-BIG1) using hLy6-BIG (e.g., hLy6-BIG1) antibodies or a hLy6-BIG (e.g., hLy6-BIG1) ligand may be used as a cancer treatment by inhibiting tumor cell growth, or inducing apoptosis of tumor cells.

hLy6-BIG (e.g., hLy6-BIG1) may be used as a marker for hair follicle stem cells, which would allow the development of therapies for hair loss.

Human Sca-1/Ly-6 genes of the invention may be used as therapeutic targets, as research tools to generate additional novel therapeutic targets, and/or as biomarkers, e.g., markers of human disease. One use of hLy6-BIG (e.g., hLy6-BIG1) will be as a marker for stem cells, including hematopoietic stem cells and non-hematopoietic stem cells such as normal stem cells (e.g., epithelial stem cells) and cancer stem cells. Isolation of normal stem cells may be used as a tool for developing therapies for tissue regeneration. hLy6-BIG (e.g., hLy6-BIG1) may also be used as a marker for cancer stem cells, and detected by using hLy6-BIG (e.g., hLy6-BIG1) antibodies or polynucleotides. Cancer stem cells isolated using hLy6-BIG (e.g., hLy6-BIG1) antibodies, either alone or in combination with other cancer stem cell markers, will then be subjected to genomics and proteomics analysis to generate additional cancer stem cell specific therapeutic targets. Colon, breast and lung stem cells are known to exist, and cancer stem cells from these tissues may be identified using hLy6-BIG antibodies (e.g., hLy6-BIG1 antibodies). hLy6-BIG antibodies (e.g., hLy6-BIG1 antibodies) may also be used to directly target tumor stem cells for cancer therapy. Based on previous studies of Ly-6 expression on mouse tumors, hLy6-BIG antibodies (e.g., hLy6-BIG1 antibodies) may be used to specifically target specific populations within a tumor that are highly malignant and/or highly metastatic, and that are normally resistant to standard cancer chemotherapies. hLy6-BIG (e.g., hLy6-BIG1) may also have cell-cell adhesion properties that promote tumor growth or metastasis, either through tumor cell-tumor cell binding or tumor cell-tumor microenvironment binding; hLy6-BIG antibodies (e.g., hLy6-BIG1 antibodies) could be used to disrupt these cellular interactions. hLy6-BIG (e.g., hLy6-BIG1) may also be used as a biomarker for detection of cancer, or detection of residual cancer following treatment using Ly6-BIG antibodies.

Other uses of the Ly6-BIG molecules of the invention include the following.

### Autoimmunity

Target/eliminate Ly6-BIG1 expressing activated/autoreactive T cells: Murine Sca-1 is expressed on most T cells, and is upregulated on activated CD4+ and CD8+ T cells. hLy6-BIG binding molecules such as antibodies (e.g., hLy6-BIG1 mAb) may be used to modulate, inhibit activity, or cause the destruction of CD4+ and/or CD8+ T cells that either directly or indirectly contribute to autoimmune disease such as Type 1 diabetes, Rheumatoid arthritis, Autoimmune thyroid diseases, Graves Disease, Hashimoto's Thyroiditis, Systemic Lupus Erythematosus, Multiple Sclerosis, Crohn's disease, Psoriasis, Psoriatic Arthritis, Sympathetic ophthalmitis, Autoimmune neuropathies, Autoimmune oophoritis, Autoimmune orchitis, Autoimmune Lymphoproliferative Syndrome, Antiphospholipid syndrome, Sjogren's Syndrome, Rheumatoid arthritis, Scleroderma, Lupus, Addison's Disease, Polyendocrine deficiency syndrome, Polyendocrine deficiency syndrome type 1, Polyendocrine deficiency syndrome type 2, Guillain-Barre Syndrome, Immune Thrombocytopenic Purpura, Pernicious anemia, Myasthenia Gravis, Primary biliary cirrhosis, Mixed connective tissue disease, Primary Glomerulonephritis, Vitiligo, Autoimmune uveitis, Autoimmune Hemolytic Anemia, Autoimmune Thrombocytopenia, Celiac Disease, Dermatitis herpetiformis, Autoimmune Hepatitis, Pemphigus, Pemphigus Vulgaris, Pemphigus Foliaceus, Bullous Pemphigoid, Autoimmune Myocarditis, Autoimmune Vasculitis, Autoimmune eye diseases, Alopecia Areata, Autoimmune Atherosclerosis, Behcet's Disease, Autoimmune Myelopathy, Autoimmune Hemophilia, Autoimmune Interstitial Cystitis, Autoimmune Diabetes Insipidus, Autoimmune Endometriosis, Relapsing Polychondritis, Ankylosing Spondylitis, Autoimmune Urticaria, Paraneoplastic Autoimmune Syndromes, Dermatomyositis, Miller Fisher Syndrome, IgA nephropathy, Goodpasture syndrome, Herpes gestationis.

Thus, Ly6-BIG molecules of the invention (e.g., Ly6-BIG binding molecules including antibodies) may be used to diagnose, prognose, and/or treat autoimmune disorders such as those described above.

Upregulate Ly6-BIG1 on regulatory T cells, to increase regulatory T cell function: Upregulation of Ly6-BIG1 may be used as an immunosuppresive therapy, to treat autoimmune disease (such as those listed above or below) or to prevent organ rejection after organ transplantation. Mouse CD3+/CD4−/CD8− double negative (DN) T cells highly express Ly-6A/Sca-1. Downregulation of Ly-6A on these DN T cells with IL-10 significantly reduced the function of DN T cells by blocking DN T cell-mediated killing. Ly-6A deficient mice showed accelerated allograft rejection compared to wild type controls (Zhang et al., Eur. J. Immunol., 32(6):1584-1592 (2002)), suggesting that ms Ly6A is critical for the immunosuppressive function of regulatory T cells. As such, human Ly6-BIG genes such as Ly6-BIG1 may also be critical in function of regulatory T cells in humans. Upregulation of Ly6-BIG1 by cytokine stimulation (eg. IFN, TNF or IL-1), by gene transfer, or by injection of Ly6-BIG1 transduced T cells could be used to increase immunosuppression to treat autoimmune diseases or to prevent organ rejection after organ transplantation.

Downregulation of Ly6-BIG1 expression on activated/autoreactive T cells by gene knockdown using Ly6-BIG1 binding molecules, e.g. RNAi, anti-sense: Mutations/antisense oligonucleotides that decrease Ly6A expression diminish T cell responsiveness (Flood et al., J. Exp. Med. 172: 115-120 (1990)). Therefore, inhibiting the expression of Ly6-BIG genes (e.g. Ly6-BIG1) with binding molecules, e.g., anti-sense oligonucleotides or RNAi may also be used to develop therapies against autoimmune disorders or other diseases caused by hyperresponsive or activated T cells.

### Cancer/Infectious Disease/AIDS:

Activating T cells as an immunotherapy against tumor or viral targets, either alone or in combination with anti-CD3/TCR antibodies and/or vaccination with tumor/viral antigens: Mouse Ly-6A functions as a co-stimulatory receptor during T cell activation via the CD3/TCR. (McGrew J T, Rock K L, Cell. Immunol. 1991, 137(1):118-26). Authors in this study showed that human Jurkat T cells transfected with mouse Ly-6A/Sca-1 could be activated by anti-ms Ly-6A specific antibody crosslinking. This study demonstrates that T cell activation via the Ly6A receptor is conserved in mouse and human, and indicates that antibody crosslinking of a human Ly6A receptor such as hLy6-BIG1 would lead to T cell activation. This could be used to develop immunotherapies to treat diseases such as cancer and infectious disease, or to boost the immune system in patients suffering from immunodeficiencies such as ME's. Antibody crosslinking of T cells may be used in combination with other T cell activating agents, such as anti-CD3/TCR and also in combination with antigenic stimulation, i.e. vaccination with tumor or viral antigens.

Upregulating or increasing Ly6-BIG1 expression on T cells, leading to enhanced activation of T cells to target tumor/viral targets: Alternatively, increasing Ly6A expression may be used to increase T cell responsiveness, and lead to the development of immunotherapies for cancer and infectious diseases. Ly6A is upregulated upon treatment with IFN-γ and IFN-α/β, TNF-α and IL-1 (Dumont, J. Immunol. 139:4088, 1987; Dumont: Eur. J. Immunol. 16: 735, 1986; Altmeyer, Cell. Immunol. 138:94, 1991). Therefore therapies using antibodies, Fc fusion proteins or ligands to Ly6-BIG1 or Ly6-BIG variants may be combined in an additive, synergistic or modulatory manner with cytokines such as IFNα,-β,-γ, TNF or IL-1 treatment to activate T cells to develop immunotherapies for cancer and infectious disease.

The Ly6-BIG molecules of the invention (e.g., polynucleotides, polypeptides, antibodies) are useful in potential diagnostic, prognostic, and therapeutic applications implicated in various diseases and disorders described herein and/or other pathologies, e.g., autoimmune disorders, cancer, and AIDS.

For example, compositions comprising the molecules of the invention will have efficacy for treatment of patients suffering from: adrenoleukodystrophy, congenital adrenal hyperplasia, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, growth and reproductive disorders, systemic lupus erythematosus, autoimmunme disease, asthma, emphysema, scleroderma, allergy, AIDS, ARDS and other diseases, disorders and conditions of the like.

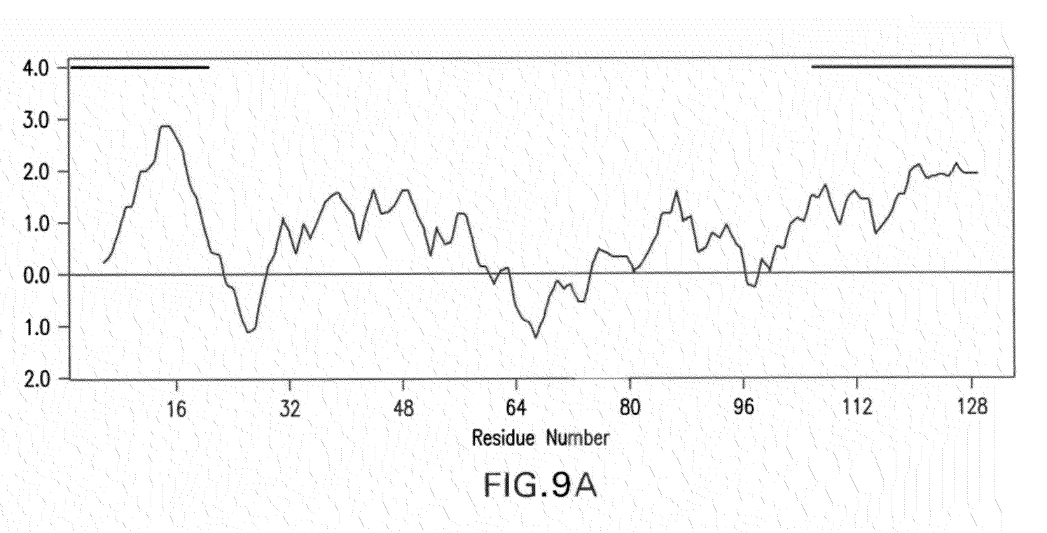

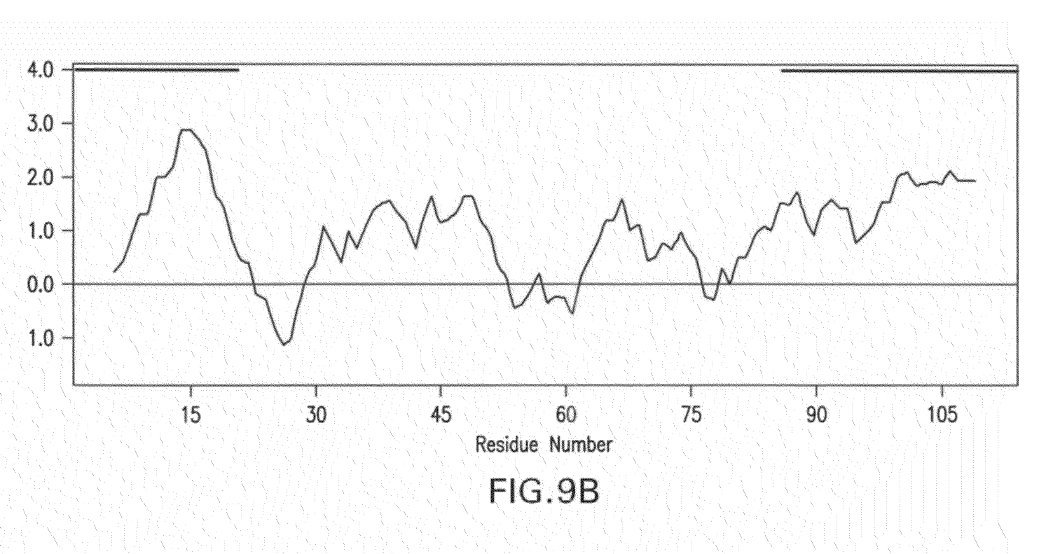

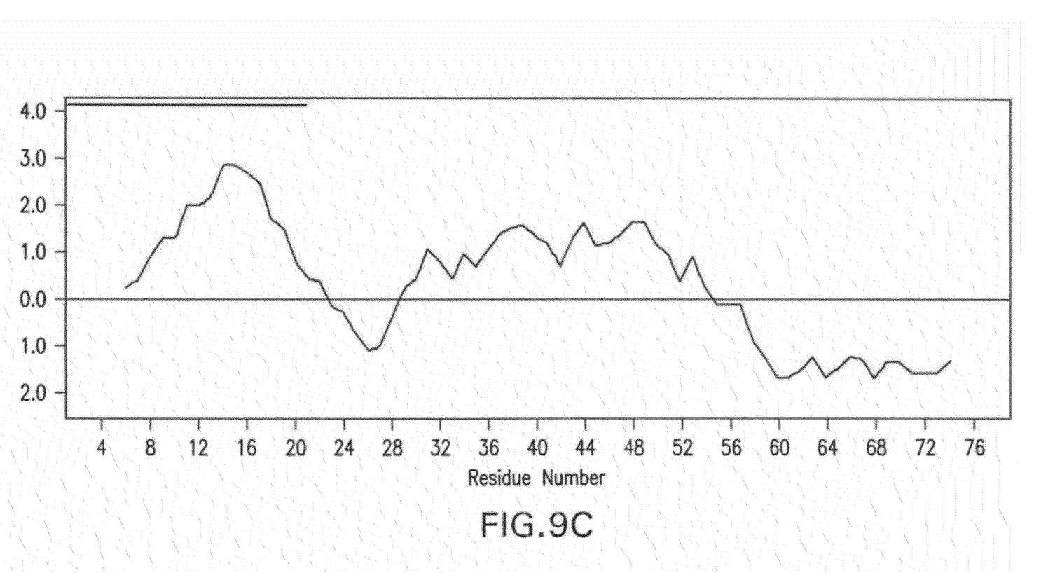

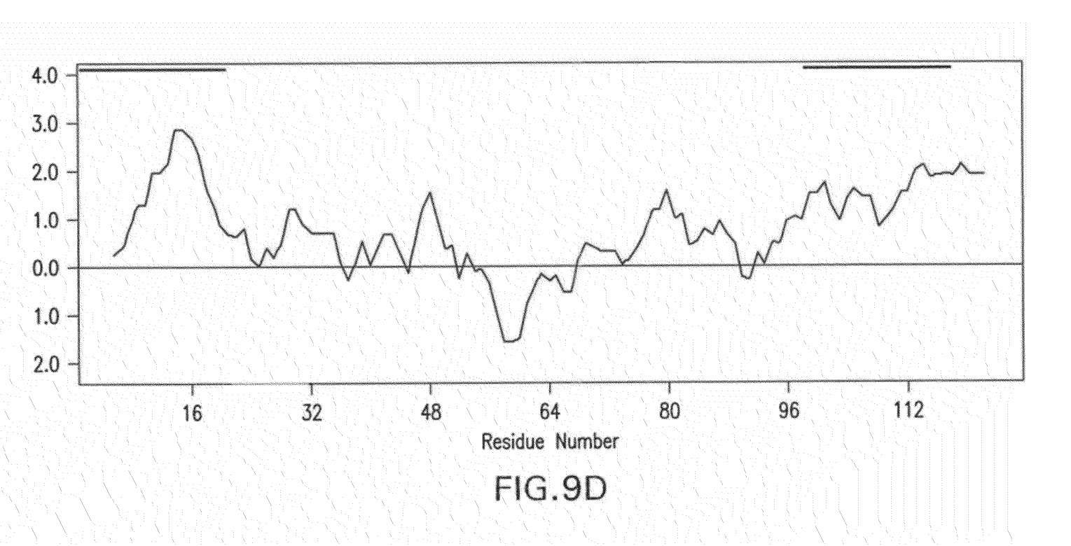

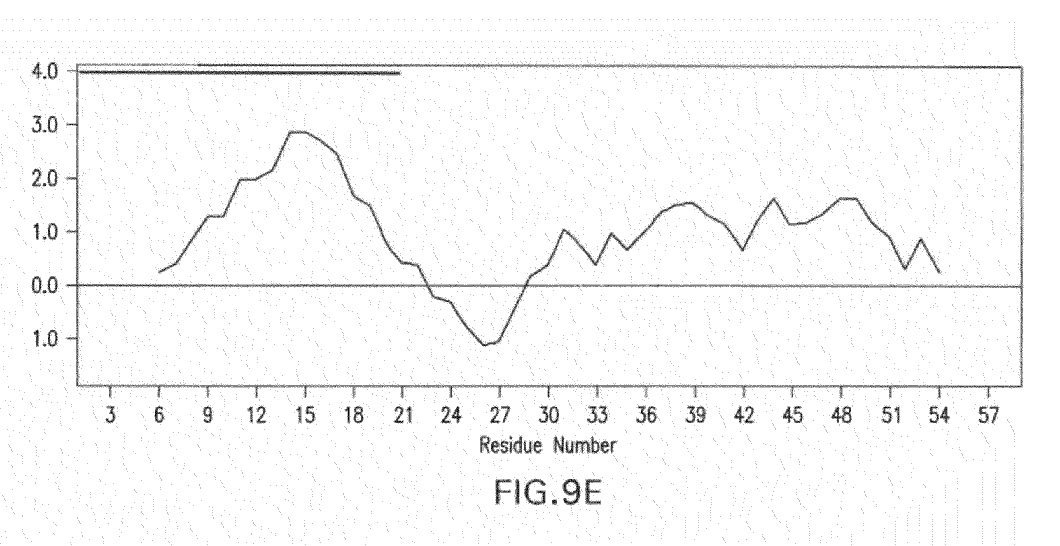

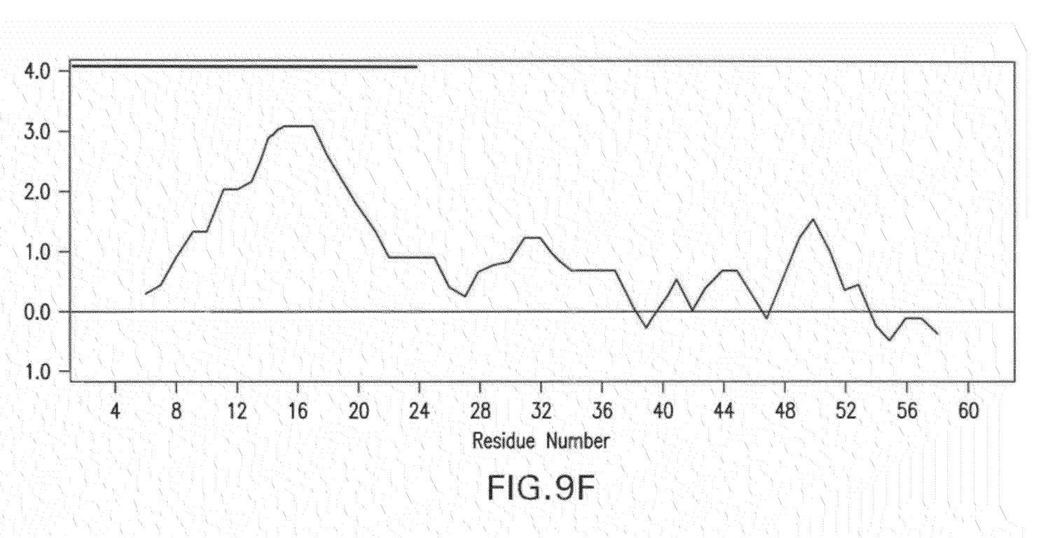

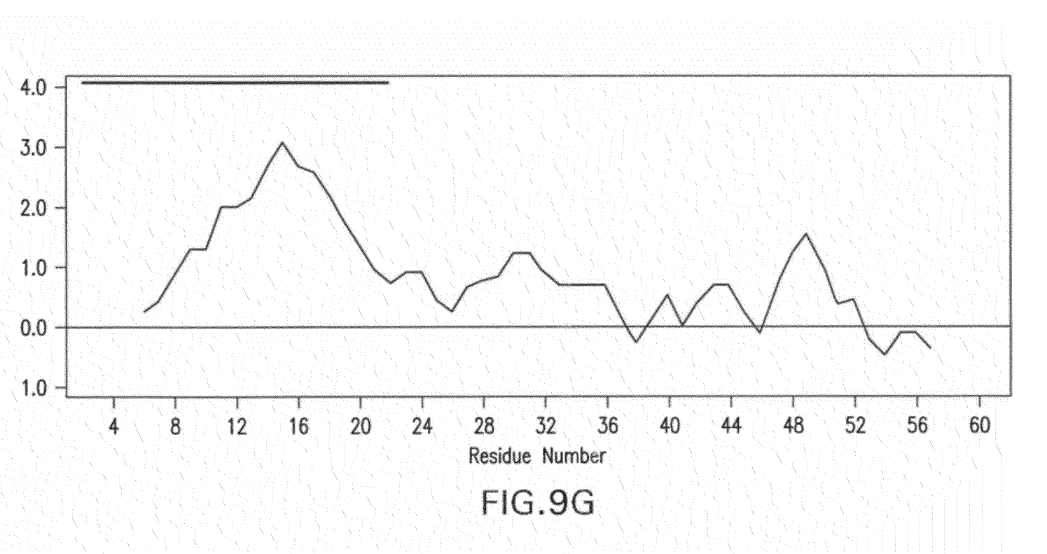

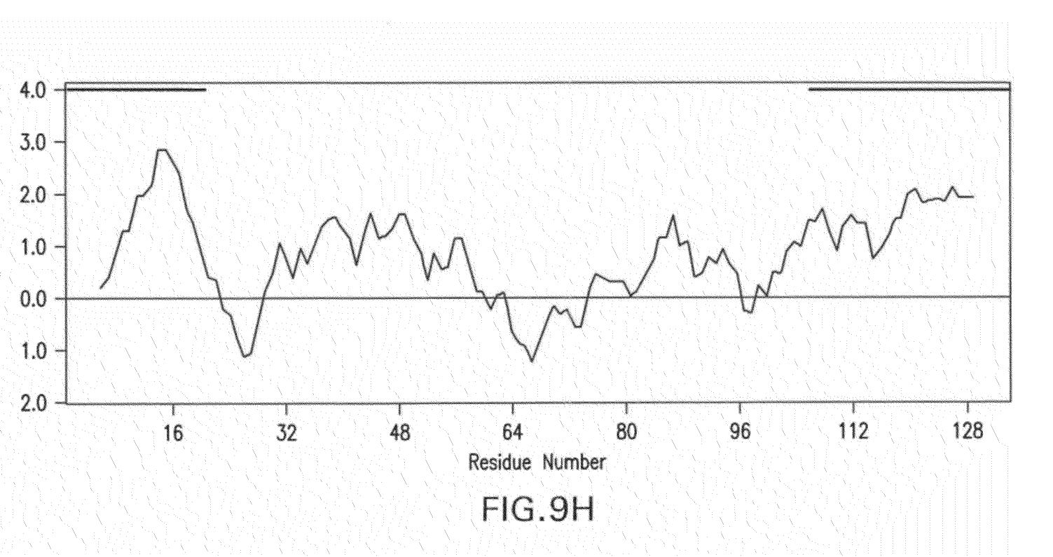

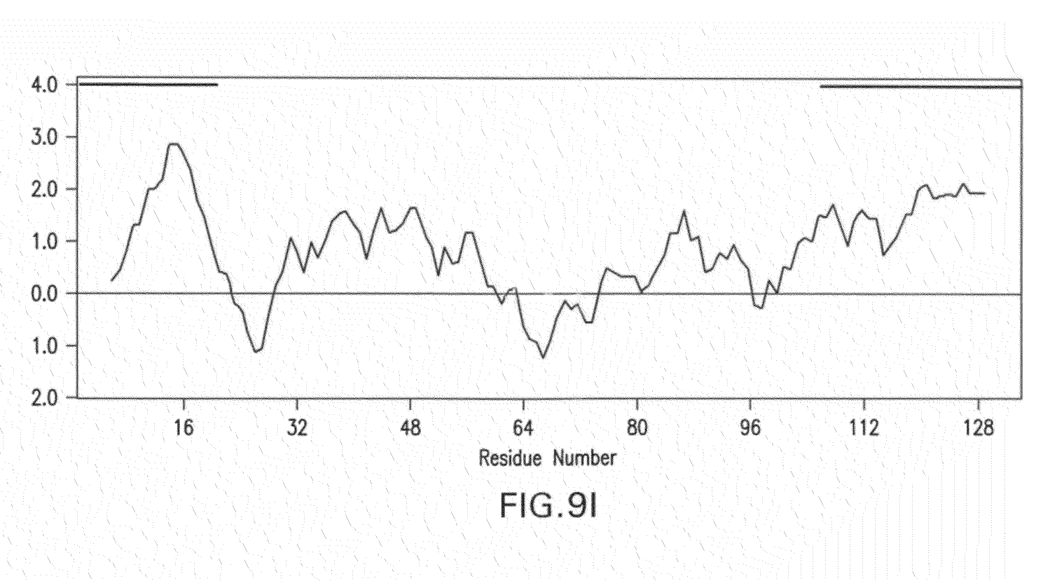

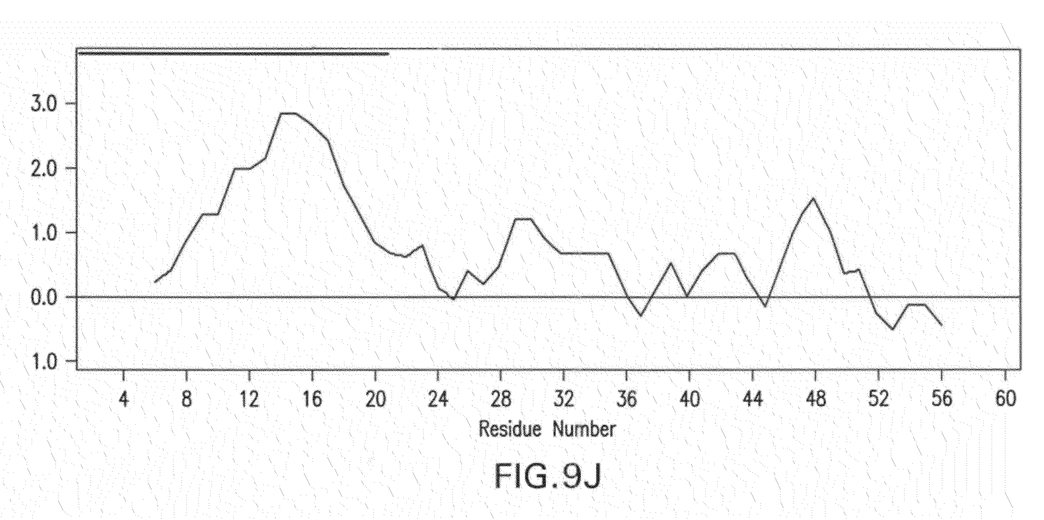

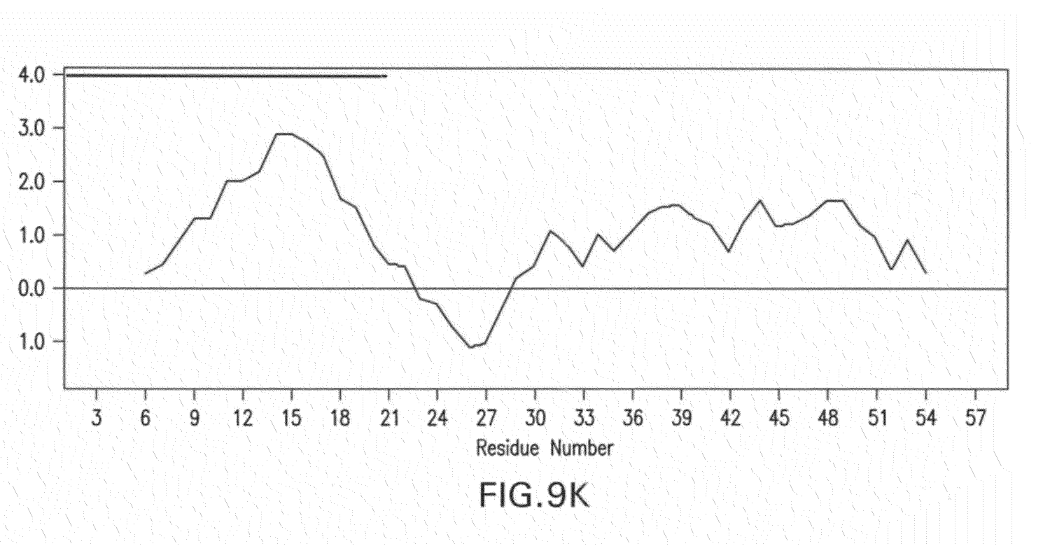

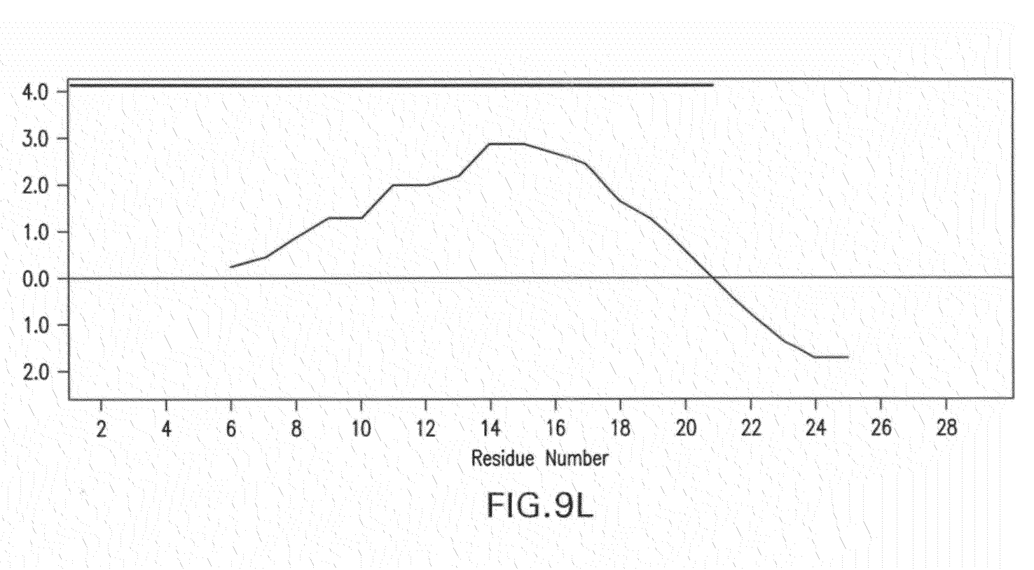

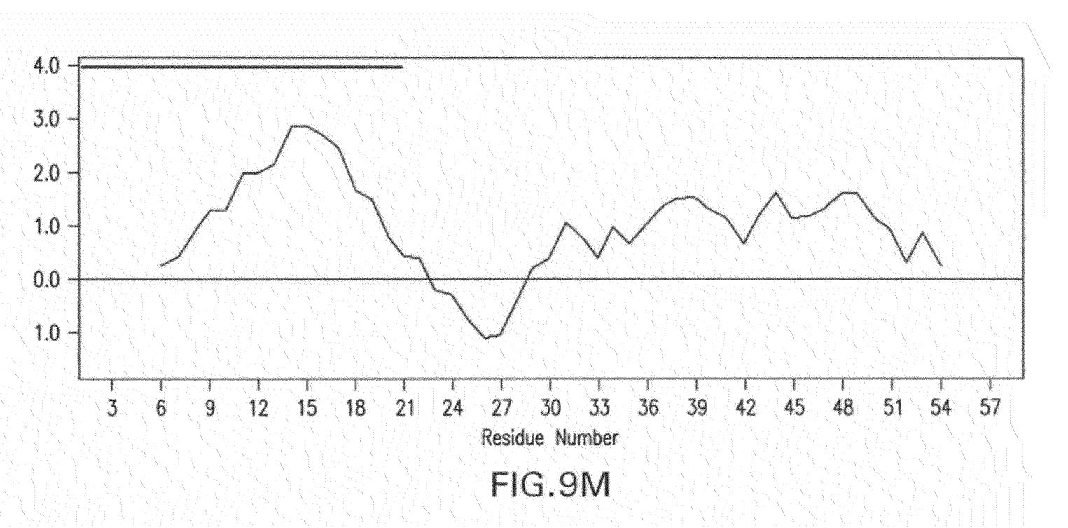

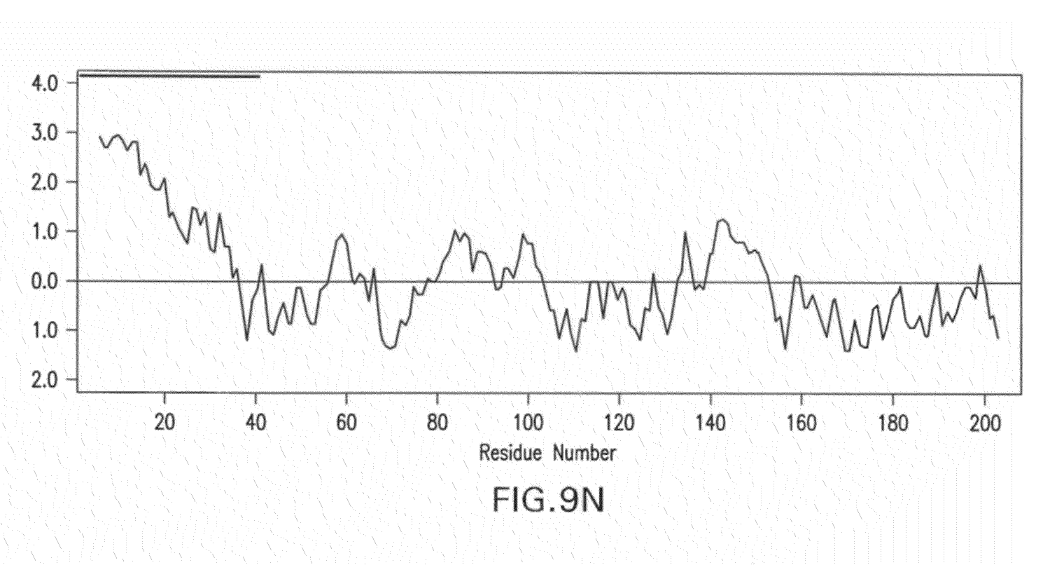

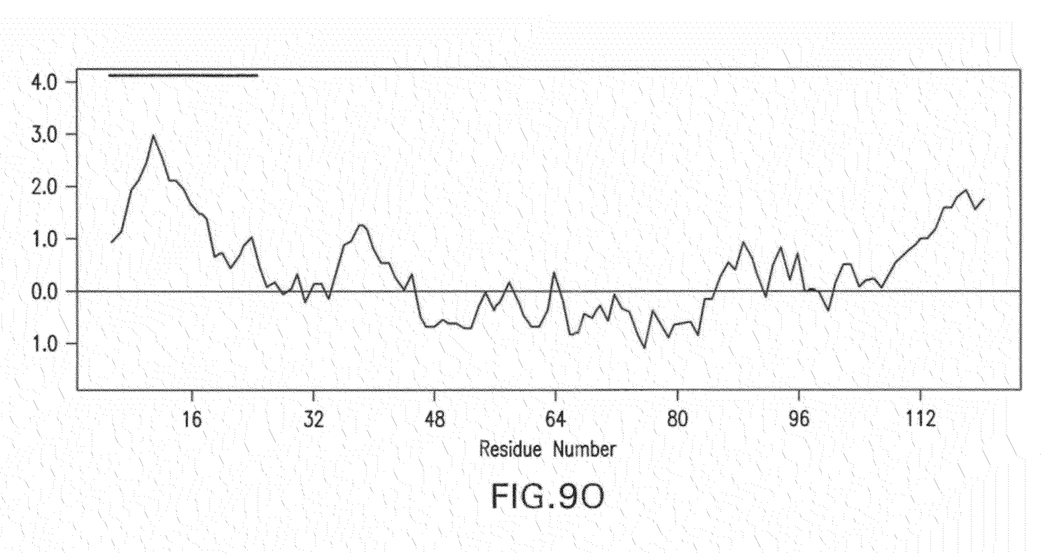

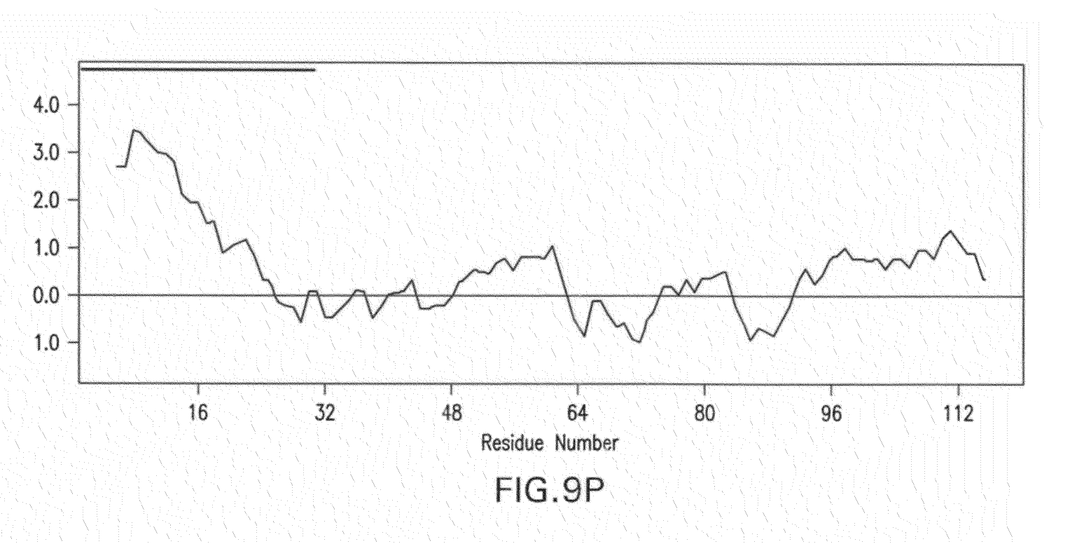

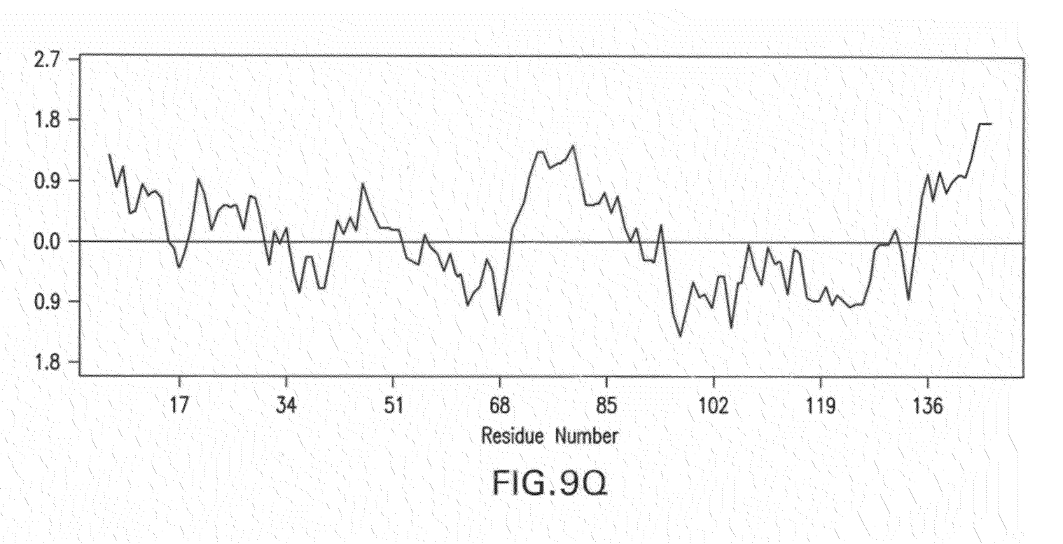

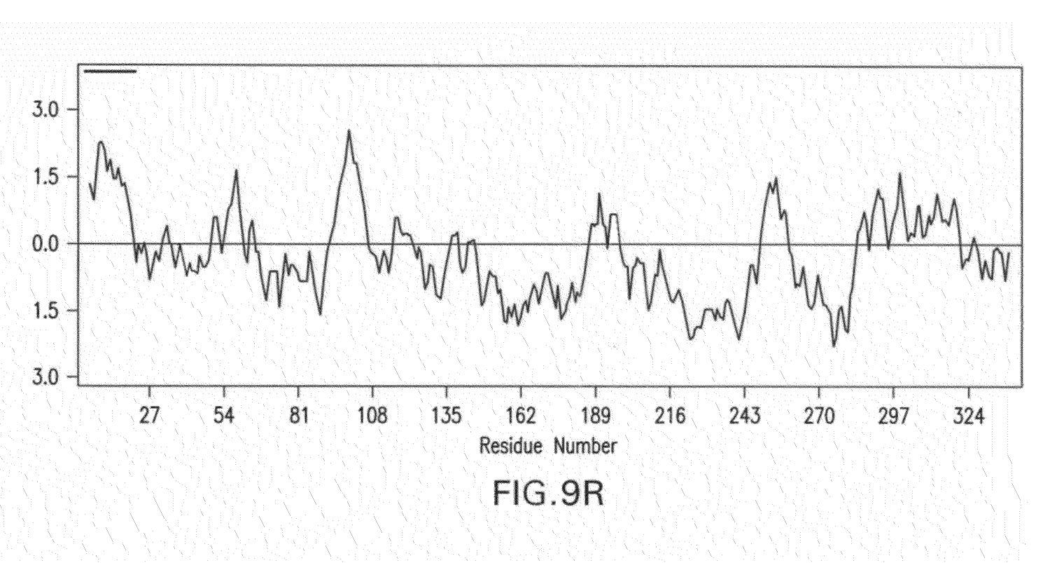

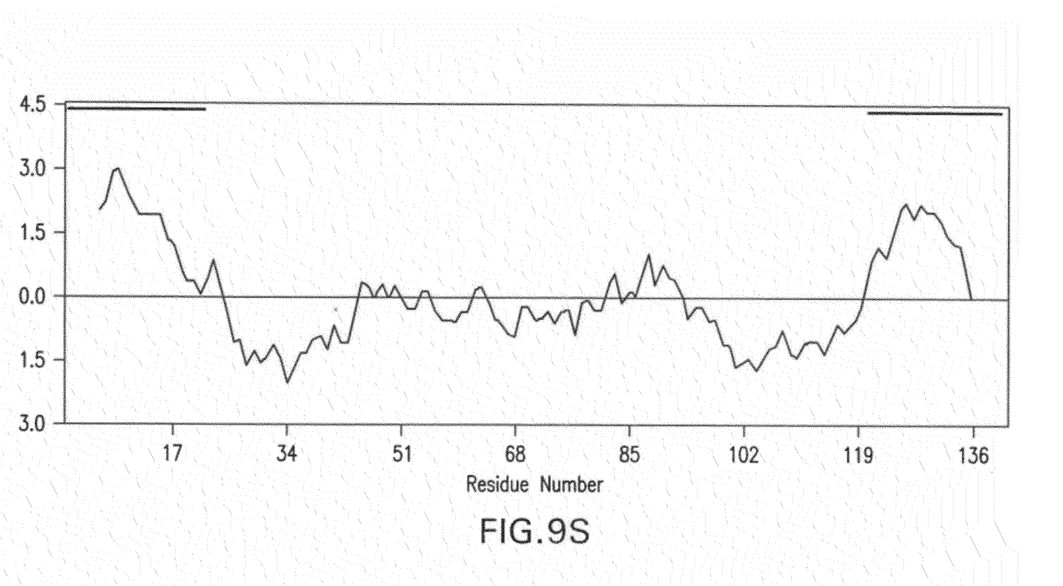

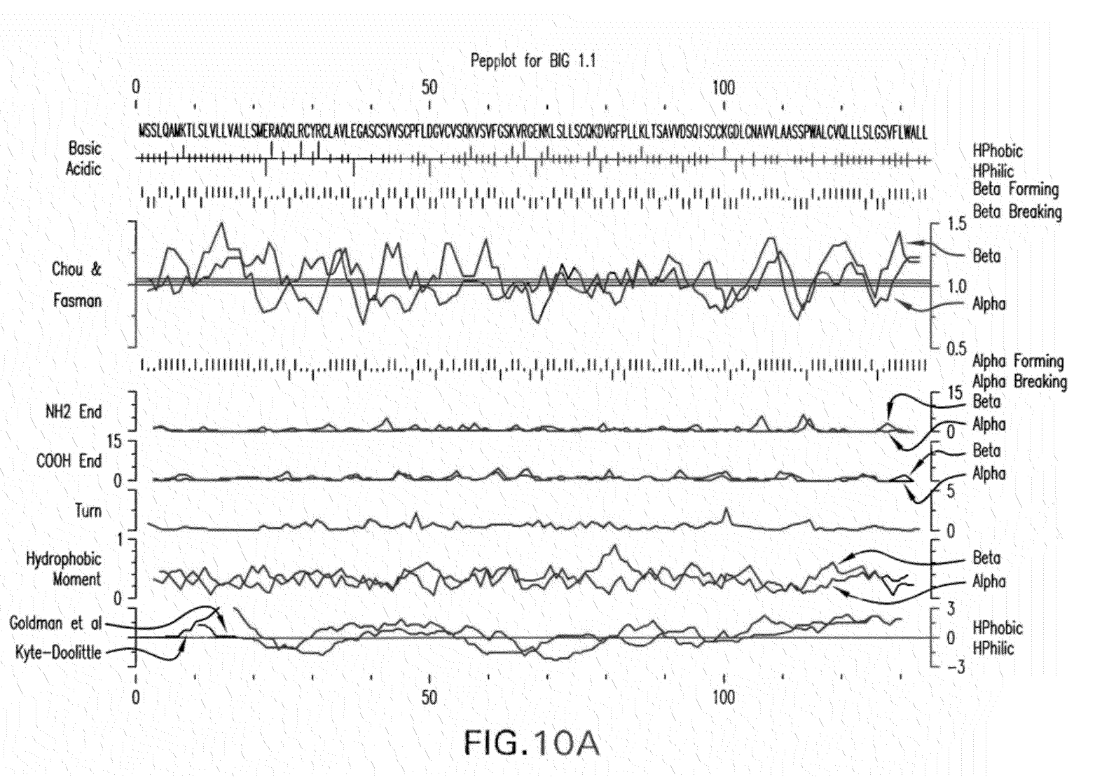

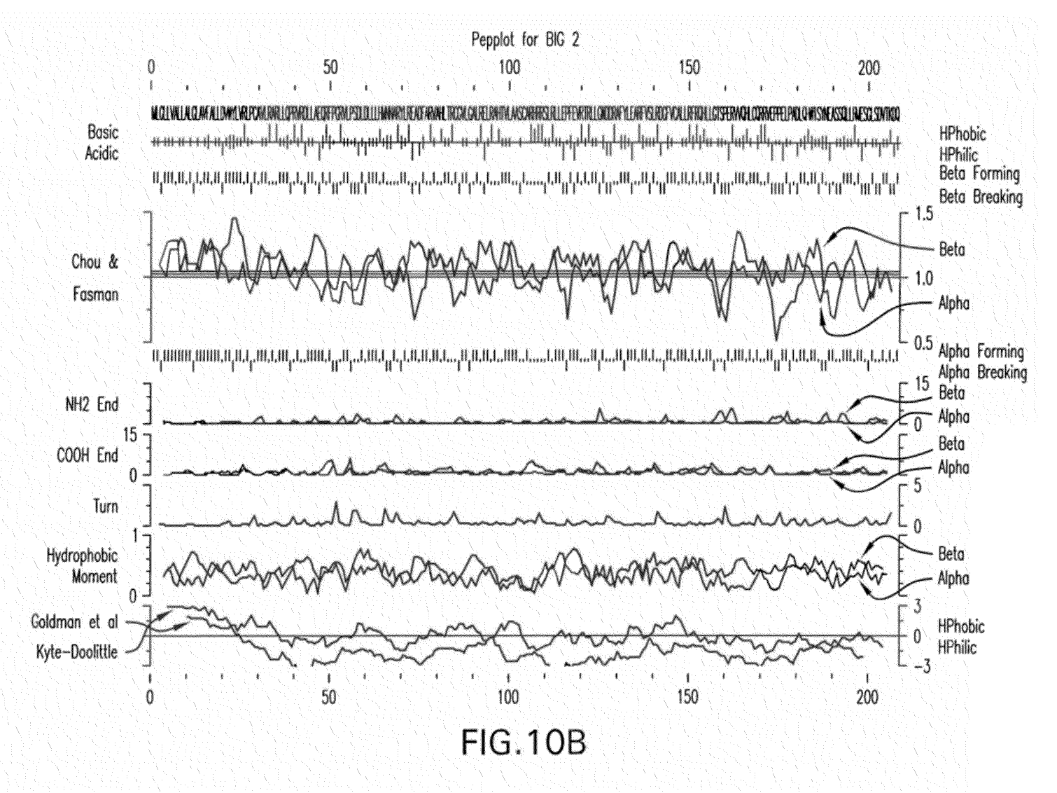

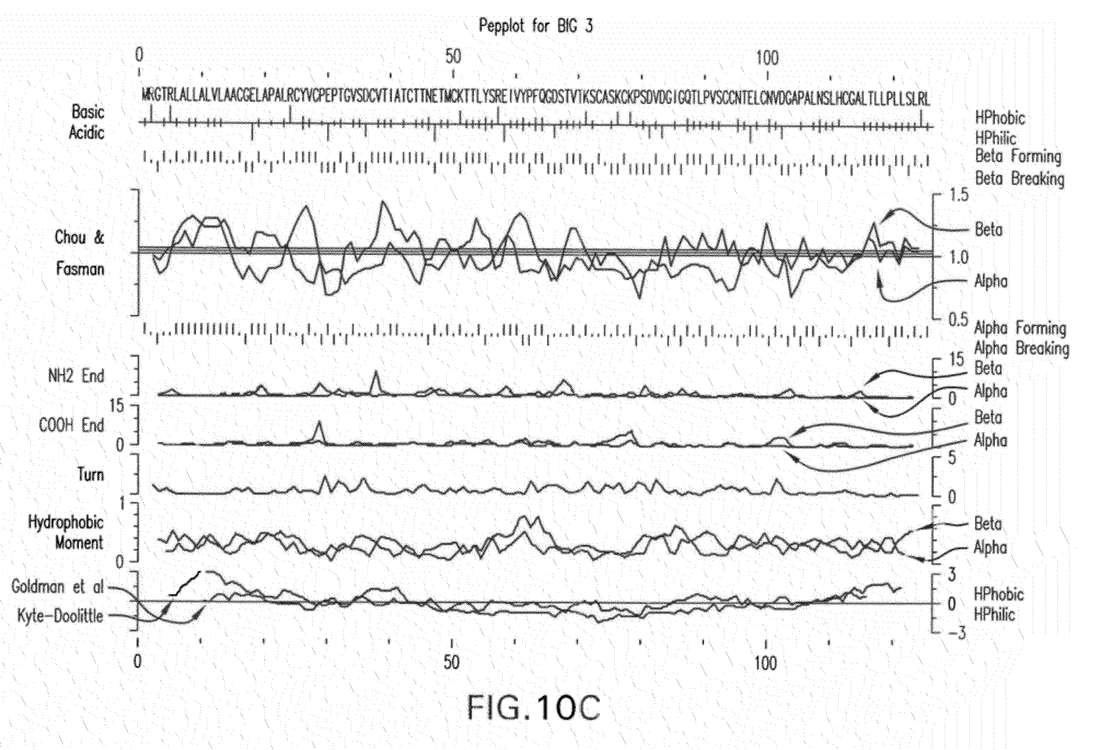

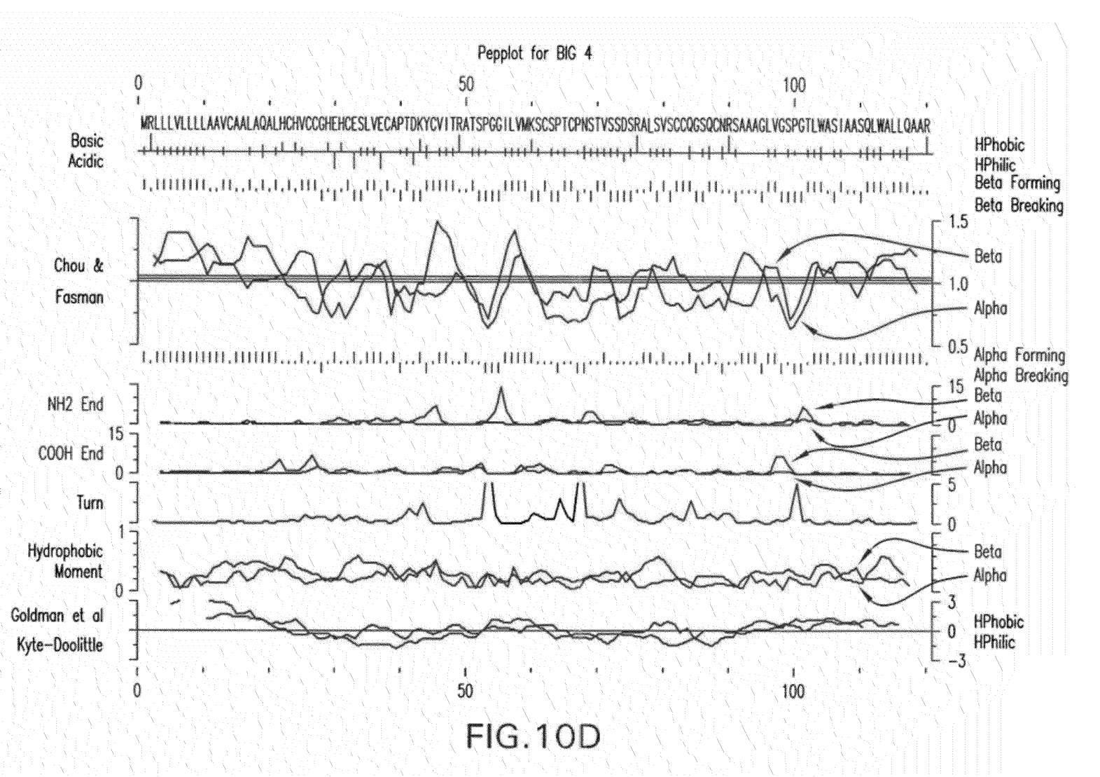

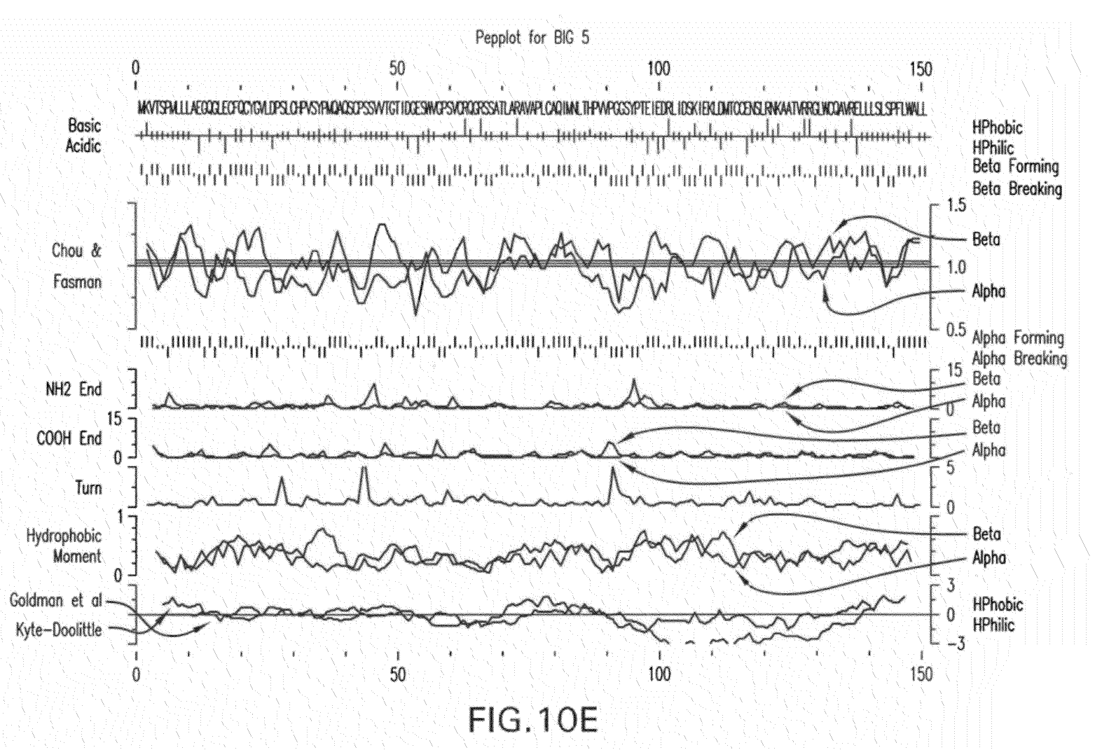

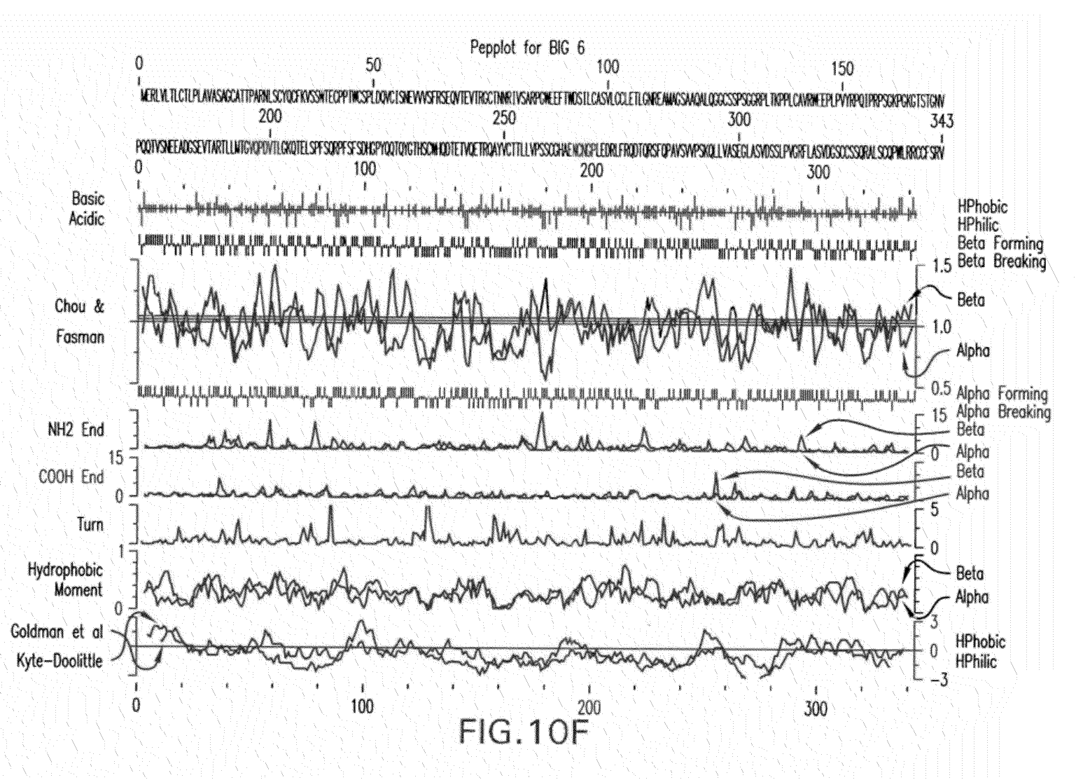

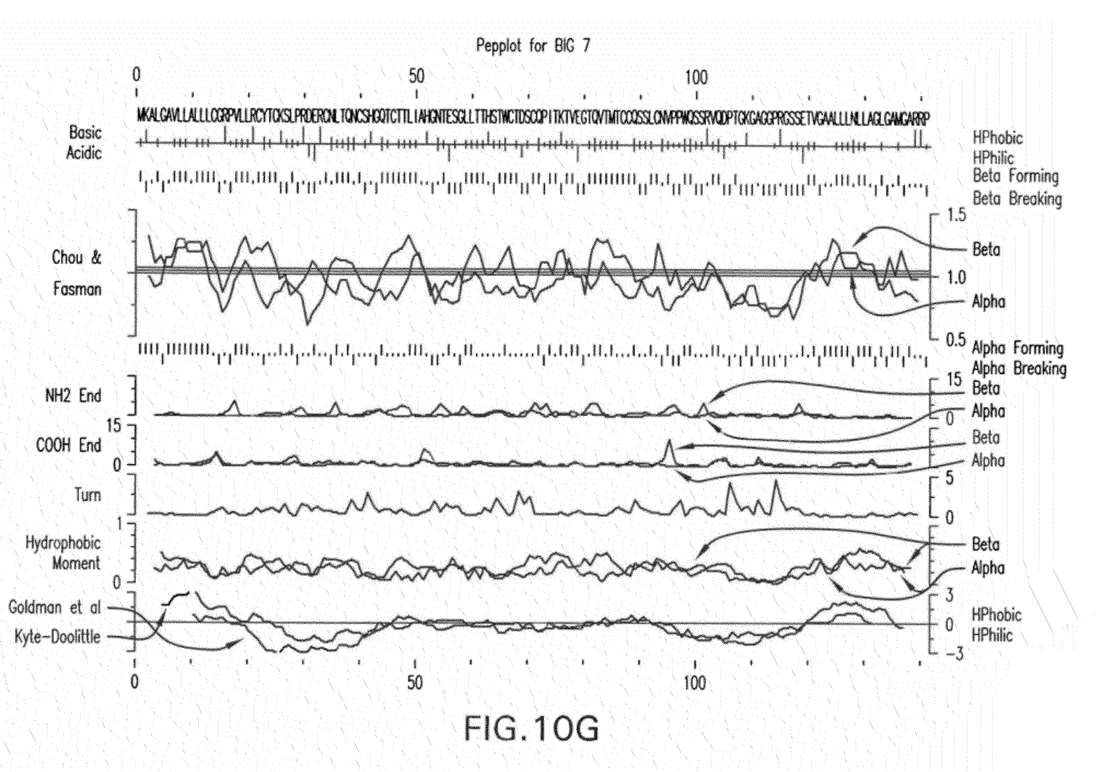

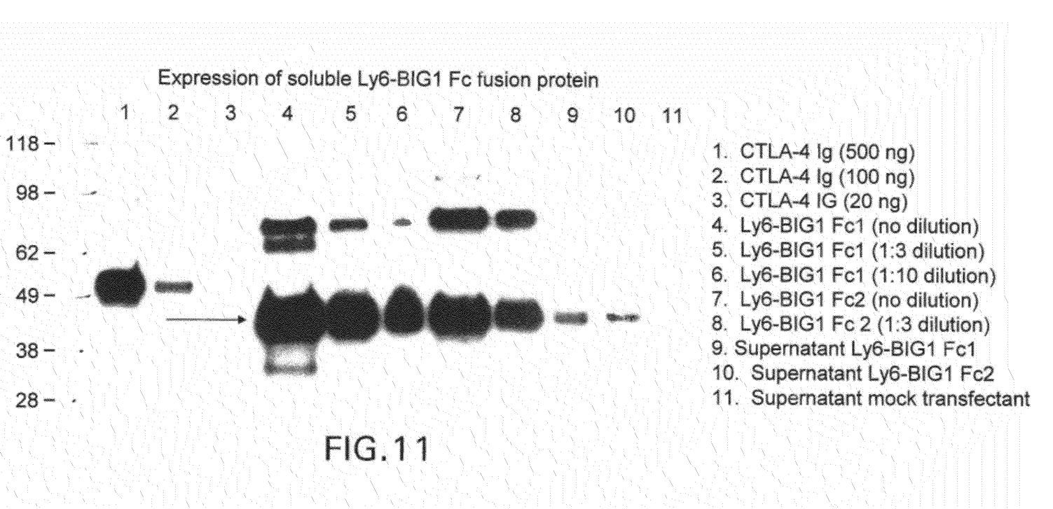

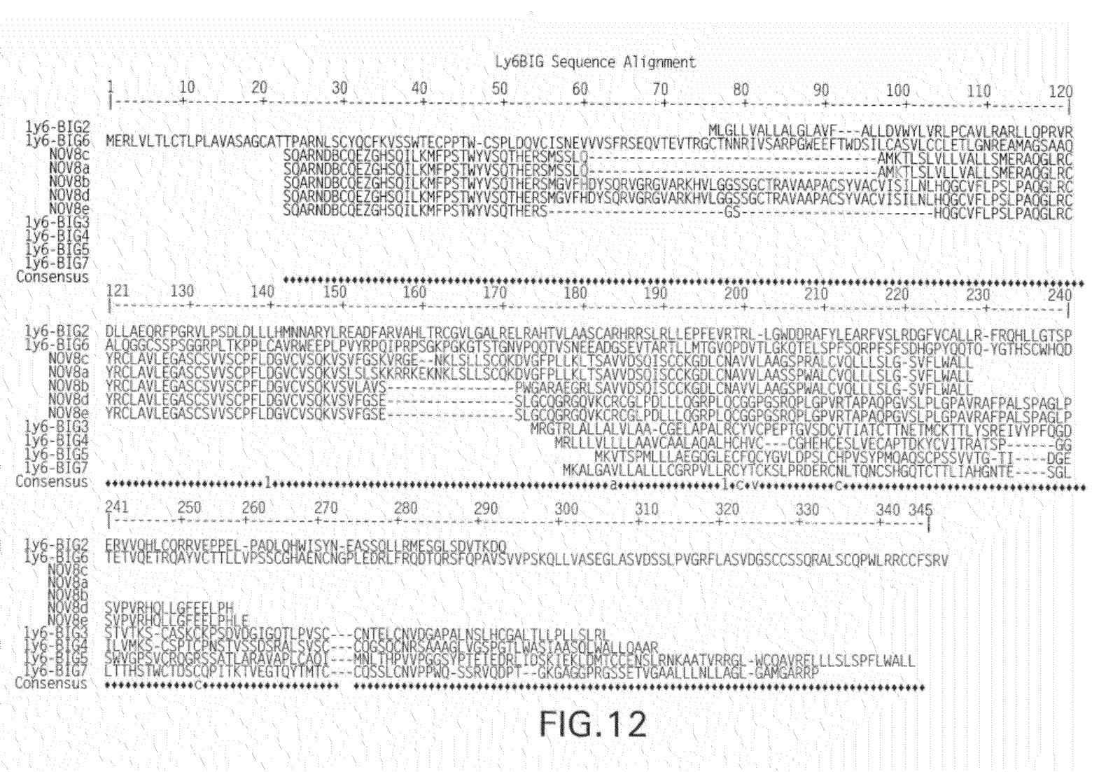

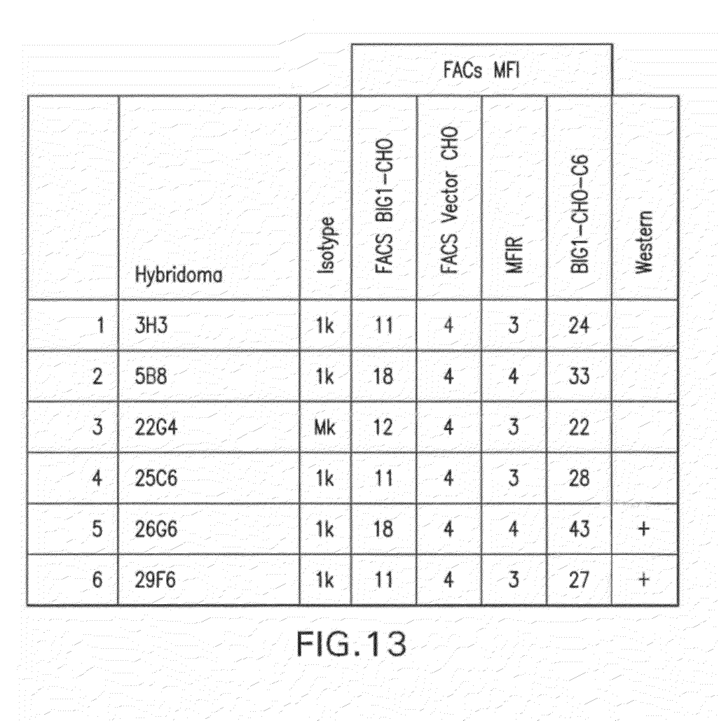

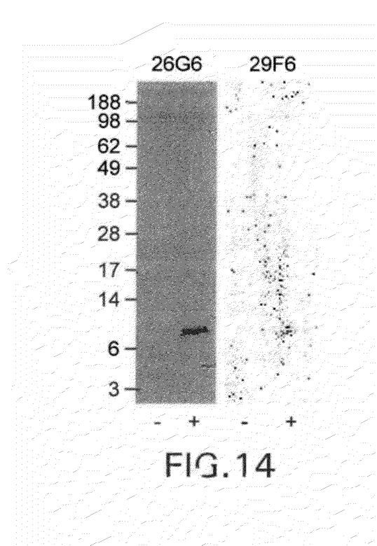

The Ly6 nucleic acids and polypeptides of the invention are further useful in the generation of antibodies that bind immunospecifically to the novel substances of the invention for use in therapeutic or diagnostic methods. These antibodies may be generated according to methods known in the art, for example, using prediction from hydrophobicity charts, as described below. For example the disclosed Ly6-BIG proteins have multiple hydrophilic regions, each of which can be used as an immunogen. See, e.g., Table 22 and FIGS. 9A-9S and 10A-10G. The novel proteins also are useful in the development of powerful assay systems for functional analysis of various human disorders, which will help in understanding of pathology of the disease and in developing new drug targets for various disorders.

For each of the uses for the human Ly6-BIG molecules described herein, hLy6-BIG molecules (e.g., antibodies) may be used in combination with each other, and/or with other therapies, and/or with stem cell specific antibodies, and/or with stem cell detecting dyes such as Hoechst 33342.

### Polypeptides of the Invention

Polypeptides of the invention include, but are not limited to, polypeptides comprising, or alternatively consisting of, an amino acid sequence of any of Tables 1-19 (SEQ ID NOS: 2, 4, 6, 8, 10, 12, 14, 16, 18, 20, 22, 24, 26, 28, 30, 32, 34, 36, or 38), polypeptides comprising, or alternatively consisting of, a polypeptide encoded by a nucleotide sequence of any of Tables 1-19 (SEQ ID NOS:1, 3, 5, 7, 9, 11, 13, 15, 17, 19, 21, 23, 25, 27, 29, 31, 33, 35, or 37), polypeptides comprising, or alternatively consisting of, a polypeptide encoded by a nucleotide sequence of one of the deposited clones, and/or mutants, fragments (e.g., portions), and variants thereof. As described below, the invention also includes polynucleotides encoding such polypeptides.

As described above, and further described below, polypeptides of the invention also include, but are not limited to, polypeptides comprising, or alternatively consisting of, mutant hLy6-BIG proteins which comprise one or more substitutions corresponding to an amino acid residue(s) of an amino acid sequence of any of Tables 1-19 (SEQ ID NOS: 2, 4, 6, 8, 10, 12, 14, 16, 18, 20, 22, 24, 26, 28, 30, 32, 34, 36, or 38), polypeptides comprising, or alternatively consisting of, mutant hLy6-BIG proteins which comprise one or more substitutions (e.g., one, two, three, four, five, six, seven, eight, nine, ten, etc.) corresponding to an amino acid residue(s) of a polypeptide encoded by a nucleotide sequence of any of Tables 1-19 (SEQ ID NOS:1, 3, 5, 7, 9, 11, 13, 15, 17, 19, 21, 23, 25, 27, 29, 31, 33, 35, or 37), polypeptides comprising, or alternatively consisting of, mutant hLy6-BIG proteins which comprise one or more substitutions (e.g., one, two, three, four, five, six, seven, eight, nine, ten, etc.) corresponding to an amino acid residue(s) of a polypeptide encoded by a nucleotide sequence of one of the deposited clones, and/or mutants, fragments (e.g., portions), and variants thereof. As described below, the invention also includes polynucleotides encoding such polypeptides.

The nucleotide sequences of Tables 1-19 (SEQ ID NOS:1, 3, 5, 7, 9, 11, 13, 15, 17, 19, 21, 23, 25, 27, 29, 31, 33, 35, or 37) and the translated amino acid sequences of Tables 1-19 (SEQ ID NOS: 2, 4, 6, 8, 10, 12, 14, 16, 18, 20, 22, 24, 26, 28, 30, 32, 34, 36, or 38) are sufficiently accurate and otherwise suitable for a variety of uses well known in the art and described further below. For instance, the nucleotide sequences of Tables 1-19 (SEQ ID NOS:1, 3, 5, 7, 9, 11, 13, 15, 17, 19, 21, 23, 25, 27, 29, 31, 33, 35, or 37) are useful for designing nucleic acid hybridization probes/primers that will detect and/or amplify nucleic acid sequences contained in SEQ ID NOS: 1, 3, 5, 7, 9, 11, 13, 15, 17, 19, 21, 23, 25, 27, 29, 31, 33, 35, or 37, respectively, or the DNAs contained in the respective deposited clone. These probes/primers will also hybridize to/amplify nucleic acid molecules in tissue or cell samples, thereby enabling detection of cells expressing SEQ ID NOS:1, 3, 5, 7, 9, 11, 13, 15, 17, 19, 21, 23, 25, 27, 29, 31, 33, 35, or 37. Similarly, polypeptides identified from SEQ ID NOS: 2, 4, 6, 8, 10, 12, 14, 16, 18, 20, 22, 24, 26, 28, 30, 32, 34, 36, or 38 may be used, for example, to generate antibodies which bind specifically to the polypeptides of the invention.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NOS:1, 3, 5, 7, 9, 11, 13, 15, 17, 19, 21, 23, 25, 27, 29, 31, 33, 35, or 37 and the predicted translated amino acid sequence identified as SEQ ID NOS: 2, 4, 6, 8, 10, 12, 14, 16, 18, 20, 22, 24, 26, 28, 30, 32, 34, 36, or 38, but also a sample of plasmid DNA containing a DNA clone encoding the hLy6-BIG proteins of the invention deposited with the American Type Culture Collection (ATCC). The nucleotide sequence of the deposited clones can readily be determined by sequencing the deposited clones in accordance with known methods. The predicted amino acid sequences can then be verified from such deposits. Moreover, the amino acid sequence of the protein encoded by the deposited clone can also be directly determined by peptide sequencing or by expressing the protein in a suitable host cell containing the deposited DNA, collecting the protein, and determining its sequence.

Polypeptides of the invention include polypeptides comprising or consisting of fragments of the polypeptides of hLy6-BIGTables 1-19 (SEQ ID NOS: 2, 4, 6, 8, 10, 12, 14, 16, 18, 20, 22, 24, 26, 28, 30, 32, 34, 36, or 38), and fragments of the hLy6-BIG proteins encoded by the deposited clones. Polypeptide fragments of the invention may be employed for producing the corresponding full-length polypeptide by peptide synthesis, therefore, the fragments may be employed as intermediates for producing the full-length polypeptides. Polypeptide fragments of the invention may also be employed for generating antibody, as described herein.

Polypeptide fragments of the invention may be from 6 to 342 amino acids in length. Thus, fragments may be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, or 342 amino acids in length. In many instances, these polypeptides fragments comprise or consist of amino acid sequences set out in one or more of Tables 1-19 with or without the N-terminal Met residue and with or without the signal sequence and with or without the signal sequence.

Polypeptide fragments of the invention may be, for example, at least 10 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, or 334 of the full length hLy6-BIG protein (e.g., the polypeptides of Tables 1-19 (SEQ ID NOS:2, 4, 6, 8, 10, 12, 14, 16, 18, 20, 22, 24, 26, 28, 30, 32, 34, 36, or 38) with or without the N-terminal Met residue and with or without the signal sequence and with or without the signal sequence, or the hLy6-BIG proteins encoded by the deposited clones). Thus, polypeptides of the invention may comprise or consist of 10 amino acid long fragments including amino acid residues 1-10, 2-11, 3-12, . . . , 125-134 of the hLy6-BIG polypeptide of Table 1 (SEQ ID NO:2); residues 1-10, 2-11, 3-12, . . . , 105-114 of the hLy6-BIG protein of Table 2 (SEQ ID NO:4); residues 1-10, 2-11, 3-12, . . . , 70-79 of the hLy6-BIG protein of Table 3 (SEQ ID NO:6); residues 1-10, 2-11, 3-12, . . . , 118-127 of the polypeptide or hLy6-BIG protein of Table 4 (SEQ ID NO:8); residues 1-10, 2-11, 3-12, . . . , 50-59 of the hLy6-BIG polypeptide of Table 5 (SEQ ID NO:10); residues 1-10, 2-11, 3-12, . . . , 54-63 of the hLy6-BIG polypeptide of Table 6 (SEQ ID NO:12); residues 1-10, 2-11, 3-12, . . . , 53-62 of the hLy6-BIG polypeptide of Table 7 (SEQ ID NO:14); residues 1-10, 2-11, 3-12, . . . , 125-134 of the hLy6-BIG polypeptide of Table 8 (SEQ ID NO:16); residues 1-10, 2-11, 3-12, . . . , 125-134 of the hLy6-BIG polypeptide of Table 9 (SEQ ID NO:18); residues 1-10, 2-11, 3-12, . . . , 52-61 of the hLy6-BIG polypeptide of Table 10 (SEQ ID NO:20); residues 1-10, 2-11, 3-12, . . . , 50-59 of the hLy6-BIG polypeptide of Table 11 (SEQ ID NO:22); residues 1-10, 2-11, 3-12, . . . , 21-30 of the hLy6-BIG polypeptide of Table 12 (SEQ ID NO:24); residues 1-10, 2-11, 3-12, . . . , 50-59 of the hLy6-BIG polypeptide of Table 13 (SEQ ID NO:26); residues 1-10, 2-11, 3-12, . . . , 199-208 of the hLy6-BIG polypeptide of Table 14 (SEQ ID NO:28); residues 1-10, 2-11, 3-12, . . . , 116-125 of the hLy6-BIG polypeptide of Table 15 (SEQ ID NO:30); residues 1-10, 2-11, 3-12, . . . , 111-120 of the hLy6-BIG polypeptide of Table 16 (SEQ ID NO:32); residues 1-10, 2-11, 3-12, . . . , 142-151 of the hLy6-BIG polypeptide of Table 17 (SEQ ID NO:34); residues 1-10, 2-11, 3-12, . . . , 334-343 of the hLy6-BIG polypeptide of Table 18 (SEQ ID NO:36); residues 1-10, 2-11, 3-12, . . . , 132-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 11 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 11 amino acid long fragments including amino acid residues 1-11, 2-12, 3-13, . . . , 124-134 of the hLy6-BIG polypeptide of Table 1 (SEQ ID NO:2); residues 1-11, 2-12, 3-13, . . . , 104-114 of the hLy6-BIG polypeptide of Table 2 (SEQ ID NO:4); residues 1-11, 2-12, 3-13, . . . , 69-79 of the hLy6-BIG polypeptide of Table 3 (SEQ ID NO:6); residues 1-11, 2-12, 3-13, . . . , 117-127 of the hLy6-BIG polypeptide of Table 4 (SEQ ID NO:8); residues 1-11, 2-12, 3-13, . . . , 49-59 of the hLy6-BIG polypeptide of Table 5 (SEQ ID NO:10); residues 1-11, 2-12, 3-13, . . . , 53-63 of the hLy6-BIG polypeptide of Table 6 (SEQ ID NO:12); residues 1-11, 2-12, 3-13, . . . , 52-62 of the hLy6-BIG polypeptide of Table 7 (SEQ ID NO:14); residues 1-11, 2-12, 3-13, . . . , 124-134 of the hLy6-BIG polypeptide of Table 8 (SEQ ID NO:16); residues 1-11, 2-12, 3-13, . . . , 124-134 of the hLy6-BIG polypeptide of Table 9 (SEQ ID NO:18); residues 1-11, 2-12, 3-13, . . . , 51-61 of the hLy6-BIG polypeptide of Table 10 (SEQ ID NO:20); residues 1-11, 2-12, 3-13, . . . , 49-59 of the hLy6-BIG polypeptide of Table 11 (SEQ ID NO:22); residues 1-11, 2-12, 3-13, . . . , 20-30 of the hLy6-BIG polypeptide of Table 12 (SEQ ID NO:24); residues 1-11, 2-12, 3-13, . . . , 49-59 of the hLy6-BIG polypeptide of Table 13 (SEQ ID NO:26); residues 1-11, 2-12, 3-13, . . . , 198-208 of the hLy6-BIG polypeptide of Table 14 (SEQ ID NO:28); residues 1-11, 2-12, 3-13, . . . , 115-125 of the hLy6-BIG polypeptide of Table 15 (SEQ ID NO:30); residues 1-11, 2-12, 3-13, . . . , 110-120 of the hLy6-BIG polypeptide of Table 16 (SEQ ID NO:32); residues 1-11, 2-12, 3-13, . . . , 141-151 of the hLy6-BIG polypeptide of Table 17 (SEQ ID NO:34); residues 1-11, 2-12, 3-13, . . . , 333-343 of the hLy6-BIG polypeptide of Table 18 (SEQ ID NO:36); residues 1-11, 2-12, 3-13, . . . , 131-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 12 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, or 332 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 12 amino acid long fragments including amino acid residues 1-12, 2-13, 3-14, . . . , 123-134 of the hLy6-BIG polypeptide of Table 1 (SEQ ID NO:2); residues 1-12, 2-13, 3-14, . . . , 103-114 of the hLy6-BIG polypeptide of Table 2 (SEQ ID NO:4); residues 1-12, 2-13, 3-14, . . . , 68-79 of the hLy6-BIG polypeptide of Table 3 (SEQ ID NO:6); residues 1-12, 2-13, 3-14, . . . , 116-127 of the hLy6-BIG polypeptide of Table 4 (SEQ ID NO:8); residues 1-12, 2-13, 3-14, . . . , 48-59 of the hLy6-BIG polypeptide of Table 5 (SEQ ID NO:10); residues 1-12, 2-13, 3-14, . . . , 52-63 of the hLy6-BIG polypeptide of Table 6 (SEQ ID NO:12); residues 1-12, 2-13, 3-14, . . . , 51-62 of the hLy6-BIG polypeptide of Table 7 (SEQ ID NO:14); residues 1-12, 2-13, 3-14, . . . , 123-134 of the hLy6-BIG polypeptide of Table 8 (SEQ ID NO:16); residues 1-12, 2-13, 3-14, . . . , 123-134 of the hLy6-BIG polypeptide of Table 9 (SEQ ID NO:18); residues 1-12, 2-13, 3-14, . . . , 50-61 of the hLy6-BIG polypeptide of Table 10 (SEQ ID NO:20); residues 1-12, 2-13, 3-14, . . . , 48-59 of the hLy6-BIG polypeptide of Table 11 (SEQ ID NO:22); residues 1-12, 2-13, 3-14, . . . , 19-30 of the hLy6-BIG polypeptide of Table 12 (SEQ ID NO:24); residues 1-12, 2-13, 3-14, . . . , 48-59 of the hLy6-BIG polypeptide of Table 13 (SEQ ID NO:26); residues 1-12, 2-13, 3-14, . . . , 197-208 of the hLy67BIG polypeptide of Table 14 (SEQ ID NO:28); residues 1-12, 2-13, 3-14, . . . , 114-125 of the hLy6-BIG polypeptide of Table 15 (SEQ ID NO:30); residues 1-12, 2-13, 3-14, . . . , 109-120 of the hLy6-BIG polypeptide of Table 16 (SEQ ID NO:32); residues 1-12, 2-13, 3-14, . . . , 140-151 of the hLy6-BIG polypeptide of Table 17 (SEQ ID NO:34); residues 1-12, 2-13, 3-14, . . . , 332-343 of the hLy6-BIG polypeptide of Table 18 (SEQ ID NO:36); residues 1-12, 2-13, 3-14, . . . , 130-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 13 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, or 331 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 13 amino acid long fragments including amino acid residues 1-13, 2-14, 3-15, . . . , 122-134 of the hLy6-BIG polypeptide of Table 1 (SEQ ID NO:2); residues 1-13, 2-14, 3-15, . . . , 102-114 of the hLy6-BIG polypeptide of Table 2 (SEQ ID NO:4); residues 1-13, 2-14, 3-15, . . . , 67-79 of the hLy6-BIG polypeptide of Table 3 (SEQ ID NO:6); residues 1-13, 2-14, 3-15, . . . , 115-127 of the hLy6-BIG polypeptide of Table 4 (SEQ ID NO:8); residues 1-13, 2-14, 3-15, . . . , 47-59 of the hLy6-BIG polypeptide of Table 5 (SEQ ID NO:10); residues 1-13, 2-14, 3-15, . . . , 51-63 of the hLy6-BIG polypeptide of Table 6 (SEQ ID NO:12); residues 1-13, 2-14, 3-15, . . . , 50-62 of the hLy6-BIG polypeptide of Table 7 (SEQ ID NO:14); residues 1-13, 2-14, 3-15, . . . , 122-143 of the hLy6-BIG polypeptide of Table 8 (SEQ ID NO:16); residues 1-13, 2-14, 3-15, . . . , 122-143 of the hLy6-BIG polypeptide of Table 9 (SEQ ID NO:18); residues 1-13, 2-14, 3-15, . . . , 49-61 of the hLy6-BIG polypeptide of Table 10 (SEQ ID NO:20); residues 1-13, 2-14, 3-15, . . . , 47-59 of the hLy6-BIG polypeptide of Table 11 (SEQ ID NO:22); residues 1-13, 2-14, 3-15, . . . , 18-30 of the hLy6-BIG polypeptide of Table 12 (SEQ ID NO:24); residues 1-13, 2-14, 3-15, . . . , 47-59 of the hLy6-BIG polypeptide of Table 13 (SEQ ID NO:26); residues 1-13, 2-14, 3-15, . . . , 196-208 of the hLy6-BIG polypeptide of Table 14 (SEQ ID NO:28); residues 1-13, 2-14, 3-15, . . . , 113-125 of the hLy6-BIG polypeptide of Table 15 (SEQ ID NO:30); residues 1-13, 2-14, 3-15, . . . , 108-120 of the hLy6-BIG polypeptide of Table 16 (SEQ ID NO:32); residues 1-13, 2-14, 3-15, . . . , 139-151 of the hLy6-BIG polypeptide of Table 17 (SEQ ID NO:34); residues 1-13, 2-14, 3-15, . . . , 331-343 of the hLy6-BIG polypeptide of Table 18 (SEQ ID NO:36); residues 1-13, 2-14, 3-15, . . . , 129-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 14 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294; 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, or 330 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 14 amino acid long fragments including amino acid residues 1-14, 2-15, 3-16, . . . , 121-134 of the hLy6-BIG polypeptide of Table 1 (SEQ ID NO:2); residues 1-14, 2-15, 3-16, . . . , 101-114 of the hLy6-BIG polypeptide of Table 2 (SEQ ID NO:4); residues 1-14, 2-15, 3-16, . . . , 66-79 of the hLy6-BIG polypeptide of Table 3 (SEQ ID NO:6); residues 1-14, 2-15, 3-16, . . . , 114-127 of the hLy6-BIG polypeptide of Table 4 (SEQ ID NO:8); residues 1-14, 2-15, 3-16, . . . , 46-59 of the hLy6-BIG polypeptide of Table 5 (SEQ ID NO:10); residues 1-14, 2-15, 3-16, . . . , 50-63 of the hLy6-BIG polypeptide of Table 6 (SEQ ID NO:12); residues 1-14, 2-15, 3-16, . . . , 49-62 of the hLy6-BIG polypeptide of Table 7 (SEQ ID NO:14); residues 1-14, 2-15, 3-16, . . . , 121-134 of the hLy6-BIG polypeptide of Table 8 (SEQ ID NO:16); residues 1-14, 2-15, 3-16, . . . , 121-134 of the hLy6-BIG polypeptide of Table 9 (SEQ ID NO:18); residues 1-14, 2-15, 3-16, . . . , 48-61 of the hLy6-BIG polypeptide of Table 10 (SEQ ID NO:20); residues 1-14, 2-15, 3-16, . . . , 46-59 of the hLy6-BIG polypeptide of Table 11 (SEQ ID NO:22); residues 1-14, 2-15, 3-16, . . . , 17-30 of the hLy6-BIG polypeptide of Table 12 (SEQ ID NO:24); residues 1-14, 2-15, 3-16, . . . , 46-59 of the hLy6-BIG polypeptide of Table 13 (SEQ ID NO:26); residues 1-14, 2-15, 3-16, . . . , 195-208 of the hLy6-BIG polypeptide of Table 14 (SEQ ID NO:28); residues 1-14, 2-15, 3-16, . . . , 112-125 of the hLy6-BIG polypeptide of Table 15 (SEQ ID NO:30); residues 1-14, 2-15, 3-16, . . . , 107-120 of the hLy6-BIG polypeptide of Table 16 (SEQ ID NO:32); residues 1-14, 2-15, 3-16, . . . , 138-151 of the hLy6-BIG polypeptide of Table 17 (SEQ ID NO:34); residues 1-14, 2-15, 3-16, . . . , 330-343 of the hLy6-BIG polypeptide of Table 18 (SEQ ID NO:36); residues 1-14, 2-15, 3-16, . . . , 128-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 15 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, or 329 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 15 amino acid long fragments including amino acid residues 1-15, 2-16, 3-17, . . . , 120-134 of the hLy6-BIG polypeptide of Table 1 (SEQ ID NO:2); residues 1-15, 2-16, 3-17, . . . , 100-114 of the hLy6-BIG polypeptide of Table 2 (SEQ ID NO:4); residues 1-15, 2-16, 3-17, . . . , 65-79 of the hLy6-BIG polypeptide of Table 3 (SEQ ID NO:6); residues 1-15, 2-16, 3-17, . . . , 113-127 of the hLy6-BIG polypeptide of Table 4 (SEQ ID NO:8); residues 1-15, 2-16, 3-17, . . . , 45-59 of the hLy6-BIG polypeptide of Table 5 (SEQ ID NO:10); residues 1-15, 2-16, 3-17, . . . , 49-63 of the hLy6-BIG polypeptide of Table 6 (SEQ ID NO:12); residues 1-15, 2-16, 3-17, . . . , 48-62 of the hLy6-BIG polypeptide of Table 7 (SEQ ID NO:14); residues 1-15, 2-16, 3-17, . . . , 120-134 of the hLy6-BIG polypeptide of Table 8 (SEQ ID NO:16); residues 1-15, 2-16, 3-17, . . . , 120-134 of the hLy6-BIG polypeptide of Table 9 (SEQ ID NO:18); residues 1-15, 2-16, 3-17, . . . , 47-61 of the hLy6-BIG polypeptide of Table 10 (SEQ ID NO:20); residues 1-15, 2-16, 3-17, . . . , 45-59 of the hLy6-BIG polypeptide of Table 11 (SEQ ID NO:22); residues 1-15, 2-16, 3-17, . . . , 16-30 of the hLy6-BIG polypeptide of Table 12 (SEQ ID NO:24); residues 1-15, 2-16, 3-17, . . . , 45-59 of the hLy6-BIG polypeptide of Table 13 (SEQ ID NO:26); residues 1-15, 2-16, 3-17, . . . , 194-208 of the hLy6-BIG polypeptide of Table 14 (SEQ ID NO:28); residues 1-15, 2-16, 3-17, . . . . , 111-125 of the hLy6-BIG polypeptide of Table 15 (SEQ ID NO:30); residues 1-15, 2-16, 3-17, . . . , 106-120 of the hLy6-BIG polypeptide of Table 16 (SEQ ID NO:32); residues 1-15, 2-16, 3-17, . . . , 137-151 of the hLy6-BIG polypeptide of Table 17 (SEQ ID NO:34); residues 1-15, 2-16, 3-17, . . . , 329-343 of the hLy6-BIG polypeptide of Table 18 (SEQ ID NO:36); residues 1-15, 2-16, 3-17, . . . , 127-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 16 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, or 328 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 16 amino acid long fragments including amino acid residues 1-16, 2-17, 3-18, . . . , 119-134 of the hLy6-BIG polypeptide of Table 1 (SEQ ID NO:2); residues 1-16, 2-17, 3-18, . . . , 99-114 of the hLy6-BIG polypeptide of Table 2 (SEQ ID NO:4); residues 1-16, 2-17, 3-18, . . . , 64-79 of the hLy6-BIG polypeptide of Table 3 (SEQ ID NO:6); residues 1-16, 2-17, 3-18, . . . , 112-127 of the hLy6-BIG polypeptide of Table 4 (SEQ ID NO:8); residues 1-16, 2-17, 3-18, . . . , 44-59 of the hLy6-BIG polypeptide of Table 5 (SEQ ID NO:10); residues 1-16, 2-17, 3-18, . . . , 48-63 of the hLy6-BIG polypeptide of Table 6 (SEQ ID NO:12); residues 1-16, 2-17, 3-18, . . . , 47-62 of the hLy6-BIG polypeptide of Table 7 (SEQ ID NO:14); residues 1-16, 2-17, 3-18, . . . , 119-134 of the hLy6-BIG polypeptide of Table 8 (SEQ ID NO:16); residues 1-16, 2-17, 3-18, . . . , 119-134 of the hLy6-BIG polypeptide of Table 9 (SEQ ID NO:18); residues 1-16, 2-17, 3-18, . . . , 46-61 of the hLy6-BIG polypeptide of Table 10 (SEQ ID NO:20); residues 1-16, 2-17, 3-18, . . . , 44-59 of the hLy6-BIG polypeptide of Table 11 (SEQ ID NO:22); residues 1-16, 2-17, 3-18, . . . , 15-30 of the hLy6-BIG polypeptide of Table 12 (SEQ ID NO:24); residues 1-16, 2-17, 3-18 . . . , 44-59 of the hLy6-BIG polypeptide of Table 13 (SEQ ID NO:26); residues 1-16, 2-17, 3-18, . . . , 193-208 of the hLy6-BIG polypeptide of Table 14 (SEQ ID NO:28); residues 1-16, 2-17, 3-18, . . . , 110-125 of the hLy6-BIG polypeptide of Table 15 (SEQ ID NO:30); residues 1-16, 2-17, 3-18, . . . , 105-120 of the hLy6-BIG polypeptide of Table 16 (SEQ ID NO:32); residues 1-16, 2-17, 3-18, . . . , 136-151 of the hLy6-BIG polypeptide of Table 17 (SEQ ID NO:34); residues 1-16, 2-17, 3-18, . . . , 328-343 of the hLy6-BIG polypeptide of Table 18 (SEQ ID NO:36); residues 1-16, 2-17, 3-18, . . . , 126-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 17 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, or 327 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 17 amino acid long fragments including amino acid residues 1-17, 2-18, 3-19, . . . , 118-134 of the hLy6-BIG polypeptide of Table 1 (SEQ ID NO:2); residues 1-17, 2-18, 3-19, . . . , 98-114 of the hLy6-BIG polypeptide of Table 2 (SEQ ID NO:4); residues 1-17, 2-18, 3-19, . . . , 63-79 of the hLy6-BIG polypeptide of Table 3 (SEQ ID NO:6); residues 1-17, 2-18, 3-19, . . . , 111-127 of the hLy6-BIG polypeptide of Table 4 (SEQ ID NO:8); residues 1-17, 2-18, 3-19, . . . , 43-59 of the hLy6-BIG polypeptide of Table 5 (SEQ ID NO:10); residues 1-17, 2-18, 3-19, . . . , 47-63 of the hLy6-BIG polypeptide of Table 6 (SEQ ID NO:12); residues 1-17, 2-18, 3-19, . . . , 46-62 of the hLy6-BIG polypeptide of Table 7 (SEQ ID NO:14); residues 1-17, 2-18, 3-19, . . . , 118-134 of the hLy6-BIG polypeptide of Table 8 (SEQ ID NO:16); residues 1-17, 2-18, 3-19, . . . , 118-134 of the hLy6-BIG polypeptide of Table 9 (SEQ ID NO:18); residues 1-17, 2-18, 3-19, . . . , 45-61 of the hLy6-BIG polypeptide of Table 10 (SEQ ID NO:20); residues 1-17, 2-18, 3-19, . . . , 43-59 of the hLy6-BIG polypeptide of Table 11 (SEQ ID NO:22); residues 1-17, 2-18, 3-19, . . . , 14-30 of the hLy6-BIG polypeptide of Table 12 (SEQ ID NO:24); residues 1-17, 2-18, 3-19, . . . , 43-59 of the hLy6-BIG polypeptide of Table 13 (SEQ ID NO:26); residues 1-17, 2-18, 3-19, . . . , 192-208 of the hLy6-BIG polypeptide of Table 14 (SEQ ID NO:28); residues 1-17, 2-18, 3-19, . . . , 109-125 of the hLy6-BIG polypeptide of Table 15 (SEQ ID NO:30); residues 1-17, 2-18, 3-19, . . . , 104-120 of the hLy6-BIG polypeptide of Table 16 (SEQ ID NO:32); residues 1-17, 2-18, 3-19, . . . , 135-151 of the hLy6-BIG polypeptide of Table 17 (SEQ ID NO:34); residues 1-17, 2-18, 3-19, . . . , 327-343 of the hLy6-BIG polypeptide of Table 18 (SEQ ID NO:36); residues 1-17, 2-18, 3-19, . . . , 125-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 18 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, or 326 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 18 amino acid long fragments including amino acid residues 1-18, 2-19, 3-20, . . . , 117-134 of the hLy6-BIG polypeptide of Table 1 (SEQ ID NO:2); residues 1-18, 2-19, 3-20, . . . , 97-114 of the hLy6-BIG polypeptide of Table 2 (SEQ ID NO:4); residues 1-18, 2-19, 3-20, . . . , 62-79 of the hLy6-BIG polypeptide of Table 3 (SEQ ID NO:6); residues 1-18, 2-19, 3-20, . . . , 110-127 of the hLy6-BIG polypeptide of Table 4 (SEQ ID NO:8); residues 1-18, 2-19, 3-20, . . . , 42-59 of the hLy6-BIG polypeptide of Table 5 (SEQ ID NO:10); residues 1-18, 2-19, 3-20, . . . , 46-63 of the hLy6-BIG polypeptide of Table 6 (SEQ ID NO:12); residues 1-18, 2-19, 3-20, . . . , 45-62 of the hLy6-BIG polypeptide of Table 7 (SEQ ID NO:14); residues 1-18, 2-19, 3-20, . . . , 117-134 of the hLy6-BIG polypeptide of Table 8 (SEQ ID NO:16); residues 1-18, 2-19, 3-20, . . . , 117-134 of the hLy6-BIG polypeptide of Table 9 (SEQ ID NO:18); residues 1-18, 2-19, 3-20, . . . , 44-61 of the hLy6-BIG polypeptide of Table 10 (SEQ ID NO:20); residues 1-18, 2-19, 3-20, . . . , 42-59 of the hLy6-BIG polypeptide of Table 11 (SEQ ID NO:22); residues 1-18, 2-19, 3-20, . . . , 13-30 of the hLy6-BIG polypeptide of Table 12 (SEQ ID NO:24); residues 1-18, 2-19, 3-20, . . . , 42-59 of the hLy6-BIG polypeptide of Table 13 (SEQ ID NO:26); residues 1-18, 2-19, 3-20, . . . , 191-208 of the hLy6-BIG polypeptide of Table 14 (SEQ ID NO:28); residues 1-18, 2-19, 3-20, . . . , 108-125 of the hLy6-BIG polypeptide of Table 15 (SEQ ID NO:30); residues 1-18, 2-19, 3-20, . . . , 103-120 of the hLy6-BIG polypeptide of Table 16 (SEQ ID NO:32); residues 1-18, 2-19, 3-20, . . . , 134-151 of the hLy6-BIG polypeptide of Table 17 (SEQ ID NO:34); residues 1-18, 2-19, 3-20, . . . , 326-343 of the hLy6-BIG polypeptide of Table 18 (SEQ ID NO:36); residues 1-18, 2-19, 3-20, . . . , 124-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 19 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, or 325 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 19 amino acid long fragments including amino acid residues 1-19, 2-20, 3-21, . . . , 116-134 of the hLy6-BIG polypeptide of Table 1 (SEQ ID NO:2); residues 1-19, 2-20, 3-21, . . . , 96-114 of the hLy6-BIG polypeptide of Table 2 (SEQ ID NO:4); residues 1-19, 2-20, 3-21, . . . , 61-79 of the hLy6-BIG polypeptide of Table 3 (SEQ ID NO:6); residues 1-19, 2-20, 3-21, . . . , 109-127 of the hLy6-BIG polypeptide of Table 4 (SEQ ID NO:8); residues 1-19, 2-20, 3-21, . . . , 41-59 of the hLy6-BIG polypeptide of Table 5 (SEQ ID NO:10); residues 1-19, 2-20, 3-21, . . . , 45-63 of the hLy6-BIG polypeptide of Table 6 (SEQ ID NO:12); residues 1-19, 2-20, 3-21, . . . , 44-62 of the hLy6-BIG polypeptide of Table 7 (SEQ ID NO:14); residues 1-19, 2-20, 3-21, . . . , 116-134 of the hLy6-BIG polypeptide of Table 8 (SEQ ID NO:16); residues 1-19, 2-20, 3-21, . . . , 116-134 of the hLy6-BIG polypeptide of Table 9 (SEQ ID NO:18); residues 1-19, 2-20, 3-21, . . . , 43-61 of the hLy6-BIG polypeptide of Table 10 (SEQ ID NO:20); residues 1-19, 2-20, 3-21, . . . , 41-59 of the hLy6-BIG polypeptide of Table 11 (SEQ ID NO:22); residues 1-19, 2-20, 3-21, . . . , 12-30 of the hLy6-BIG polypeptide of Table 12 (SEQ ID NO:24); residues 1-19, 2-20, 3-21, . . . , 41-59 of the hLy6-BIG polypeptide of Table 13 (SEQ ID NO:26); residues 1-19, 2-20, 3-21, . . . , 190-208 of the hLy6-BIG polypeptide of Table 14 (SEQ ID NO:28); residues 1-19, 2-20, 3-21, . . . , 107-125 of the hLy6-BIG polypeptide of Table 15 (SEQ ID NO:30); residues 1-19, 2-20, 3-21, . . . , 102-120 of the hLy6-BIG polypeptide of Table 16 (SEQ ID NO:32); residues 1-19, 2-20, 3-21, . . . , 133-151 of the hLy6-BIG polypeptide of Table 17 (SEQ ID NO:34); residues 1-19, 2-20, 3-21, . . . , 325-343 of the hLy6-BIG polypeptide of Table 18 (SEQ ID NO:36); residues 1-19, 2-20, 3-21, . . . , 123-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 20 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, or 324 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 20 amino acid long fragments including amino acid residues 1-20, 2-21, 3-22, . . . , 115-134 of the hLy6-BIG polypeptide of Table 1 (SEQ ID NO:2); residues 1-20, 2-21, 3-22, . . . , 95-114 of the hLy6-BIG polypeptide of Table 2 (SEQ ID NO:4); residues 1-20, 2-21, 3-22, . . . , 60-79 of the hLy6-BIG polypeptide of Table 3 (SEQ ID NO:6); residues 1-20, 2-21, 3-22, . . . , 108-127 of the hLy6-BIG polypeptide of Table 4 (SEQ ID NO:8); residues 1-20, 2-21, 3-22, . . . , 40-59 of the hLy6-BIG polypeptide of Table 5 (SEQ ID NO:10); residues 1-20, 2-21, 3-22, . . . , 44-63 of the hLy6-BIG polypeptide of Table 6 (SEQ ID NO:12); residues 1-20, 2-21, 3-22, . . . , 43-62 of the hLy6-BIG polypeptide of Table 7 (SEQ ID NO:14); residues 1-20, 2-21, 3-22, . . . , 115-134 of the hLy6-BIG polypeptide of Table 8 (SEQ ID NO:16); residues 1-20, 2-21, 3-22, . . . , 115-134 of the hLy6-BIG polypeptide of Table 9 (SEQ ID NO:18); residues 1-20, 2-21, 3-22, . . . , 42-61 of the hLy6-BIG polypeptide of Table 10 (SEQ ID NO:20); residues 1-20, 2-21, 3-22, . . . , 40-59 of the hLy6-BIG polypeptide of Table 11 (SEQ ID NO:22); residues 1-20, 2-21, 3-22, . . . , 11-30 of the hLy6-BIG polypeptide of Table 12 (SEQ ID NO:24); residues 1-20, 2-21, 3-22, . . . , 40-59 of the hLy6-BIG polypeptide of Table 13 (SEQ ID NO:26); residues 1-20, 2-21, 3-22, . . . , 189-208 of the hLy6-BIG polypeptide of Table 14 (SEQ ID NO:28); residues 1-20, 2-21, 3-22, . . . , 106-125 of the hLy6-BIG polypeptide of Table 15 (SEQ ID NO:30); residues 1-20, 2-21, 3-22, . . . , 101-120 of the hLy6-BIG polypeptide of Table 16 (SEQ ID NO:32); residues 1-20, 2-21, 3-22, . . . , 132-151 of the hLy6-BIG polypeptide of Table 17 (SEQ ID NO:34); residues 1-20, 2-21, 3-22, . . . , 324-343 of the hLy6-BIG polypeptide of Table 18 (SEQ ID NO:36); residues 1-20, 2-21, 3-22, . . . , 122-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 21 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, or 323 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 21 amino acid long fragments including amino acid residues 1-21, 2-22, 3-23, . . . , 114-134 of the hLy6-BIG polypeptide of Table 1 (SEQ ID NO:2); residues 1-21, 2-22, 3-23, . . . , 94-114 of the hLy6-BIG polypeptide of Table 2 (SEQ ID NO:4); residues 1-21, 2-22, 3-23, . . . , 59-79 of the hLy6-BIG polypeptide of Table 3 (SEQ ID NO:6); residues 1-21, 2-22, 3-23, . . . , 107-127 of the hLy6-BIG polypeptide of Table 4 (SEQ ID NO:8); residues 1-21, 2-22, 3-23, . . . , 39-59 of the hLy6-BIG polypeptide of Table 5 (SEQ ID NO:10); residues 1-21, 2-22, 3-23, . . . , 43-63 of the hLy6-BIG polypeptide of Table 6 (SEQ ID NO:12); residues 1-21, 2-22, 3-23, . . . , 42-62 of the hLy6-BIG polypeptide of Table 7 (SEQ ID NO:14); residues 1-21, 2-22, 3-23, . . . , 114-134 of the hLy6-BIG polypeptide of Table 8 (SEQ ID NO:16); residues 1-21, 2-22, 3-23, . . . , 114-134 of the hLy6-BIG polypeptide of Table 9 (SEQ ID NO:18); residues 1-21, 2-22, 3-23, . . . , 41-61 of the hLy6-BIG polypeptide of Table 10 (SEQ ID NO:20); residues 1-21, 2-22, 3-23, . . . , 39-59 of the hLy6-BIG polypeptide of Table 11 (SEQ ID NO:22); residues 1-21, 2-22, 3-23, . . . , 10-30 of the hLy6-BIG polypeptide of Table 12 (SEQ ID NO:24); residues 1-21, 2-22, 3-23, . . . , 39-59 of the hLy6-BIG polypeptide of Table 13 (SEQ ID NO:26); residues 1-21, 2-22, 3-23, . . . , 188-208 of the hLy6-BIG polypeptide of Table 14 (SEQ ID NO:28); residues 1-21, 2-22, 3-23, . . . , 105-125 of the hLy6-BIG polypeptide of Table 15 (SEQ ID NO:30); residues 1-21, 2-22, 3-23, . . . , 100-120 of the hLy6-BIG polypeptide of Table 16 (SEQ ID NO:32); residues 1-21, 2-22, 3-23, . . . , 131-151 of the hLy6-BIG polypeptide of Table 17 (SEQ ID NO:34); residues 1-21, 2-22, 3-23, . . . , 323-343 of the hLy6-BIG polypeptide of Table 18 (SEQ ID NO:36); residues 1-21, 2-22, 3-23, . . . , 121-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 22 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, or 322 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 22 amino acid long fragments including amino acid residues 1-22, 2-23, 3-24, . . . , 113-134 of the hLy6-BIG polypeptide of Table 1 (SEQ ID NO:2); residues 1-22, 2-23, 3-24, . . . , 93-114 of the hLy6-BIG polypeptide of Table 2 (SEQ ID NO:4); residues 1-22, 2-23, 3-24, . . . , 58-79 of the hLy6-BIG polypeptide of Table 3 (SEQ ID NO:6); residues 1-22, 2-23, 3-24, . . . , 106-127 of the hLy6-BIG polypeptide of Table 4 (SEQ ID NO:8); residues 1-22, 2-23, 3-24, . . . , 38-59 of the hLy6-BIG polypeptide of Table 5 (SEQ ID NO:10); residues 1-22, 2-23, 3-24, . . . , 42-63 of the hLy6-BIG polypeptide of Table 6 (SEQ ID NO:12); residues 1-22, 2-23, 3-24, . . . , 41-62 of the hLy6-BIG polypeptide of Table 7 (SEQ ID NO:14); residues 1-22, 2-23, 3-24, . . . , 113-134 of the hLy6-BIG polypeptide of Table 8 (SEQ ID NO:16); residues 1-22, 2-23, 3-24, . . . , 113-134 of the hLy6-BIG polypeptide of Table 9 (SEQ ID NO:18); residues 1-22, 2-23, 3-24, . . . , 40-61 of the hLy6-BIG polypeptide of Table 10 (SEQ ID NO:20); residues 1-22, 2-23, 3-24, . . . , 38-59 of the hLy6-BIG polypeptide of Table II (SEQ ID NO:22); residues 1-22, 2-23, 3-24, . . . , 9-30 of the hLy6-BIG polypeptide of Table 12 (SEQ ID NO:24); residues 1-22, 2-23, 3-24, . . . , 38-59 of the hLy6-BIG polypeptide of Table 13 (SEQ ID NO:26); residues 1-22, 2-23, 3-24, . . . , 187-208 of the hLy6-BIG polypeptide of Table 14 (SEQ ID NO:28); residues 1-22, 2-23, 3-24, . . . , 104-125 of the hLy6-BIG polypeptide of Table 15 (SEQ ID NO:30); residues 1-22, 2-23, 3-24, . . . , 99-120 of the hLy6-BIG polypeptide of Table 16 (SEQ ID NO:32); residues 1-22, 2-23, 3-24, . . . , 130-151 of the hLy6-BIG polypeptide of Table 17 (SEQ ID NO:34); residues 1-22, 2-23, 3-24, . . . , 322-343 of the hLy6-BIG polypeptide of Table 18 (SEQ ID NO:36); residues 1-22, 2-23, 3-24, . . . , 120-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 23 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, or 321 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 23 amino acid long fragments including amino acid residues 1-23, 2-24, 3-25, . . . , 112-134 of the hLy6-BIG polypeptide of Table 1 (SEQ ID NO:2); residues 1-23, 2-24, 3-25, . . . , 92-114 of the hLy6-BIG polypeptide of Table 2 (SEQ ID NO:4); residues 1-23, 2-24, 3-25, . . . , 57-79 of the hLy6-BIG polypeptide of Table 3 (SEQ ID NO:6); residues 1-23, 2-24, 3-25, . . . , 105-127 of the hLy6-BIG polypeptide of Table 4 (SEQ ID NO:8); residues 1-23, 2-24, 3-25, . . . , 37-59 of the hLy6-BIG polypeptide of Table 5 (SEQ ID NO:10); residues 1-23, 2-24, 3-25, . . . , 41-63 of the hLy6-BIG polypeptide of Table 6 (SEQ ID NO:12); residues 1-23, 2-24, 3-25, . . . , 40-62 of the hLy6-BIG polypeptide of Table 7 (SEQ ID NO:14); residues 1-23, 2-24, 3-25, . . . , 112-134 of the hLy6-BIG polypeptide of Table 8 (SEQ ID NO:16); residues 1-23, 2-24, 3-25, . . . , 112-134 of the hLy6-BIG polypeptide of Table 9 (SEQ ID NO:18); residues 1-23, 2-24, 3-25, . . . , 39-61 of the hLy6-BIG polypeptide of Table 10 (SEQ ID NO:20); residues 1-23, 2-24, 3-25, 37-59 of the hLy6-BIG polypeptide of Table 11 (SEQ ID NO:22); residues 1-23, 2-24, 3-25, . . . , 8-30 of the hLy6-BIG polypeptide of Table 12 (SEQ ID NO:24); residues 1-23, 2-24, 3-25, . . . , 37-59 of the hLy6-BIG polypeptide of Table 13 (SEQ ID NO:26); residues 1-23, 2-24, 3-25, . . . , 186-208 of the hLy6-BIG polypeptide of Table 14 (SEQ ID NO:28); residues 1-23, 2-24, 3-25, . . . , 103-125 of the hLy6-BIG polypeptide of Table 15 (SEQ ID NO:30); residues 1-23, 2-24, 3-25, . . . , 98-120 of the hLy6-BIG polypeptide of Table 16 (SEQ ID NO:32); residues 1-23, 2-24, 3-25, . . . , 129-151 of the hLy6-BIG polypeptide of Table 17 (SEQ ID NO:34); residues 1-23, 2-24, 3-25, . . . , 321-343 of the hLy6-BIG polypeptide of Table 18 (SEQ ID NO:36); residues 1-23, 2-24, 3-25, . . . , 119-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 24 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 24 amino acid long fragments including amino acid residues 1-24, 2-25, 3-26, . . . , 111-134 of the hLy6-BIG polypeptide of Table 1 (SEQ ID NO:2); residues 1-24, 2-25, 3-26, . . . , 91-114 of the hLy6-BIG polypeptide of Table 2 (SEQ ID NO:4); residues 1-24, 2-25, 3-26, . . . , 56-79 of the hLy6-BIG polypeptide of Table 3 (SEQ ID NO:6); residues 1-24, 2-25, 3-26, . . . , 104-127 of the hLy6-BIG polypeptide of Table 4 (SEQ ID NO:8); residues 1-24, 2-25, 3-26, . . . , 36-59 of the hLy6-BIG polypeptide of Table 5 (SEQ ID NO:10); residues 1-24, 2-25, 3-26, . . . , 40-63 of the hLy6-BIG polypeptide of Table 6 (SEQ ID NO:12); residues 1-24, 2-25, 3-26, . . . , 39-62 of the hLy6-BIG polypeptide of Table 7 (SEQ ID NO:14); residues 1-24, 2-25, 3-26, . . . , 111-134 of the hLy6-BIG polypeptide of Table 8 (SEQ ID NO:16); residues 1-24, 2-25, 3-26, . . . , 111-134 of the hLy6-BIG polypeptide of Table 9 (SEQ ID NO:18); residues 1-24, 2-25, 3-26, . . . , 38-61 of the hLy6-BIG polypeptide of Table 10 (SEQ ID NO:20); residues 1-24, 2-25, 3-26, . . . , 36-59 of the hLy6-BIG polypeptide of Table 11 (SEQ ID NO:22); residues 1-24, 2-25, 3-26, . . . , 7-30 of the hLy6-BIG polypeptide of Table 12 (SEQ ID NO:24); residues 1-24, 2-25, 3-26, . . . , 36-59 of the hLy6-BIG polypeptide of Table 13 (SEQ ID NO:26); residues 1-24, 2-25, 3-26, . . . , 185-208 of the hLy6-BIG polypeptide of Table 14 (SEQ ID NO:28); residues 1-24, 2-25, 3-26, . . . , 102-125 of the hLy6-BIG polypeptide of Table 15 (SEQ ID NO:30); residues 1-24, 2-25, 3-26, . . . , 97-120 of the hLy6-BIG polypeptide of Table 16 (SEQ ID NO:32); residues 1-24, 2-25, 3-26, . . . , 128-151 of the hLy6-BIG polypeptide of Table 17 (SEQ ID NO:34); residues 1-24, 2-25, 3-26, . . . , 320-343 of the hLy6-BIG polypeptide of Table 18 (SEQ ID NO:36); residues 1-24, 2-25, 3-26, . . . , 118-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 25 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, or 319 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 25 amino acid long fragments including amino acid residues 1-25, 2-26, 3-27, . . . , 110-134 of the hLy6-BIG polypeptide of Table 1 (SEQ ID NO:2); residues 1-25, 2-26, 3-27, . . . , 90-114 of the hLy6-BIG polypeptide of Table 2 (SEQ ID NO:4); residues 1-25, 2-26, 3-27, . . . , 55-79 of the hLy6-BIG polypeptide of Table 3 (SEQ ID NO:6); residues 1-25, 2-26, 3-27, . . . , 103-127 of the hLy6-BIG polypeptide of Table 4 (SEQ ID NO:8); residues 1-25, 2-26, 3-27, . . . , 35-59 of the hLy6-BIG polypeptide of Table 5 (SEQ ID NO:10); residues 1-25, 2-26, 3-27, . . . , 39-63 of the hLy6-BIG polypeptide of Table 6 (SEQ ID NO:12); residues 1-25, 2-26, 3-27, . . . , 38-62 of the hLy6-BIG polypeptide of Table 7 (SEQ ID NO:14); residues 1-25, 2-26, 3-27, . . . , 110-134 of the hLy6-BIG polypeptide of Table 8 (SEQ ID NO:16); residues 1-25, 2-26, 3-27, . . . , 110-134 of the hLy6-BIG polypeptide of Table 9 (SEQ ID NO:18); residues 1-25, 2-26, 3-27, . . . , 37-61 of the hLy6-BIG polypeptide of Table 10 (SEQ ID NO:20); residues 1-25, 2-26, 3-27, . . . , 35-59 of the hLy6-BIG polypeptide of Table 11 (SEQ ID NO:22); residues 1-25, 2-26, 3-27, . . . , 6-30 of the hLy6-BIG polypeptide of Table 12 (SEQ ID NO:24); residues 1-25, 2-26, 3-27, . . . , 35-59 of the hLy6-BIG polypeptide of Table 13 (SEQ ID NO:26); residues 1-25, 2-26, 3-27, . . . , 184-208 of the hLy6-BIG polypeptide of Table 14 (SEQ ID NO:28); residues 1-25, 2-26, 3-27, . . . , 101-125 of the hLy6-BIG polypeptide of Table 15 (SEQ ID NO:30); residues 1-25, 2-26, 3-27, . . . , 96-120 of the hLy6-BIG polypeptide of Table 16 (SEQ ID NO:32); residues 1-25, 2-26, 3-27, . . . , 127-151 of the hLy6-BIG polypeptide of Table 17 (SEQ ID NO:34); residues 1-25, 2-26, 3-27, . . . , 319-343 of the hLy6-BIG polypeptide of Table 18 (SEQ ID NO:36); residues 1-25, 2-26, 3-27, . . . , 117-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 26 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, or 318 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 26 amino acid long fragments including amino acid residues 1-26, 2-27, 3-28, . . . , 109-134 of the hLy6-BIG polypeptide of Table 1 (SEQ ID NO:2); residues 1-26, 2-27, 3-28, . . . , 89-114 of the hLy6-BIG polypeptide of Table 2 (SEQ ID NO:4); residues 1-26, 2-27, 3-28, . . . , 54-79 of the hLy6-BIG polypeptide of Table 3 (SEQ ID NO:6); residues 1-26, 2-27, 3-28, . . . , 102-127 of the hLy6-BIG polypeptide of Table 4 (SEQ ID NO:8); residues 1-26, 2-27, 3-28, . . . , 34-59 of the hLy6-BIG polypeptide of Table 5 (SEQ ID NO:10); residues 1-26, 2-27, 3-28, . . . , 38-63 of the hLy6-BIG polypeptide of Table 6 (SEQ ID NO:12); residues 1-26, 2-27, 3-28, . . . , 37-62 of the hLy6-BIG polypeptide of Table 7 (SEQ ID NO:14); residues 1-26, 2-27, 3-28, . . . , 109-134 of the hLy6-BIG polypeptide of Table 8 (SEQ ID NO:16); residues 1-26, 2-27, 3-28, . . . , 109-134 of the hLy6-BIG polypeptide of Table 9 (SEQ ID NO:18); residues 1-26, 2-27, 3-28, . . . , 36-61 of the hLy6-BIG polypeptide of Table 10 (SEQ ID NO:20); residues 1-26, 2-27, 3-28, . . . , 34-59 of the hLy6-BIG polypeptide of Table 11 (SEQ ID NO:22); residues 1-26, 2-27, 3-28, . . . , 5-30 of the hLy6-BIG polypeptide of Table 12 (SEQ ID NO:24); residues 1-26, 2-27, 3-28, . . . , 34-59 of the hLy6-BIG polypeptide of Table 13 (SEQ ID NO:26); residues 1-26, 2-27, 3-28, . . . , 183-208 of the hLy6-BIG polypeptide of Table 14 (SEQ ID NO:28); residues 1-26, 2-27, 3-28, . . . , 100-125 of the hLy6-BIG polypeptide of Table 15 (SEQ ID NO:30); residues 1-26, 2-27, 3-28, . . . , 95-120 of the hLy6-BIG polypeptide of Table 16 (SEQ ID NO:32); residues 1-26, 2-27, 3-28, . . . , 126-151 of the hLy6-BIG polypeptide of Table 17 (SEQ ID NO:34); residues 1-26, 2-27, 3-28, . . . , 318-343 of the hLy6-BIG polypeptide of Table 18 (SEQ ID NO:36); residues 1-26, 2-27, 3-28, . . . , 116-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 27 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, or 317 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 27 amino acid long fragments including amino acid residues 1-27, 2-28, 3-29, . . . , 108-134 of the hLy6-BIG polypeptide of Table 1 (SEQ ID NO:2); residues 1-27, 2-28, 3-29, . . . , 88-114 of the hLy6-BIG polypeptide of Table 2 (SEQ ID NO:4); residues 1-27, 2-28, 3-29, . . . , 53-79 of the hLy6-BIG polypeptide of Table 3 (SEQ ID NO:6); residues 1-27, 2-28, 3-29, . . . , 101-127 of the hLy6-BIG polypeptide of Table 4 (SEQ ID NO:8); residues 1-27, 2-28, 3-29, . . . , 33-59 of the hLy6-BIG polypeptide of Table 5 (SEQ ID NO:10); residues 1-27, 2-28, 3-29, . . . , 37-63 of the hLy6-BIG polypeptide of Table 6 (SEQ ID NO:12); residues 1-27, 2-28, 3-29, . . . , 36-62 of the hLy6-BIG polypeptide of Table 7 (SEQ ID NO:14); residues 1-27, 2-28, 3-29, . . . , 108-134 of the hLy6-BIG polypeptide of Table 8 (SEQ ID NO:16); residues 1-27, 2-28, 3-29, . . . , 108-134 of the hLy6-BIG polypeptide of Table 9 (SEQ ID NO:18); residues 1-27, 2-28, 3-29, . . . , 35-61 of the hLy6-BIG polypeptide of Table 10 (SEQ ID NO:20); residues 1-27, 2-28, 3-29, . . . , 33-59 of the hLy6-BIG polypeptide of Table 11 (SEQ ID NO:22); residues 1-27, 2-28, 3-29, 4-30 of the hLy6-BIG polypeptide of Table 12 (SEQ ID NO:24); residues 1-27, 2-28, 3-29, . . . , 33-59 of the hLy6-BIG polypeptide of Table 13 (SEQ ID NO:26); residues 1-27, 2-28, 3-29, . . . , 182-208 of the hLy6-BIG polypeptide of Table 14 (SEQ ID NO:28); residues 1-27, 2-28, 3-29, . . . , 99-125 of the hLy6-BIG polypeptide of Table 15 (SEQ ID NO:30); residues 1-27, 2-28, 3-29, . . . , 94-120 of the hLy6-BIG polypeptide of Table 16 (SEQ ID NO:32); residues 1-27, 2-28, 3-29, . . . , 125-151 of the hLy6-BIG polypeptide of Table 17 (SEQ ID NO:34); residues 1-27, 2-28, 3-29, . . . , 317-343 of the hLy6-BIG polypeptide of Table 18 (SEQ ID NO:36); residues 1-27, 2-28, 3-29, . . . , 115-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 28 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, or 316 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 28 amino acid long fragments including amino acid residues 1-28, 2-29, 3-30, . . . , 107-134 of the hLy6-BIG polypeptide of Table 1 (SEQ ID NO:2); residues 1-28, 2-29, 3-30, . . . , 87-114 of the hLy6-BIG polypeptide of Table 2 (SEQ ID NO:4); residues 1-28, 2-29, 3-30, . . . , 52-79 of the hLy6-BIG polypeptide of Table 3 (SEQ ID NO:6); residues 1-28, 2-29, 3-30, . . . , 100-127 of the hLy6-BIG polypeptide of Table 4 (SEQ ID NO:8); residues 1-28, 2-29, 3-30, . . . , 32-59 of the hLy6-BIG polypeptide of Table 5 (SEQ ID NO:10); residues 1-28, 2-29, 3-30, . . . , 36-63 of the hLy6-BIG polypeptide of Table 6 (SEQ ID NO:12); residues 1-28, 2-29, 3-30, . . . , 35-62 of the hLy6-BIG polypeptide of Table 7 (SEQ ID NO:14); residues 1-28, 2-29, 3-30, . . . , 107-134 of the hLy6-BIG polypeptide of Table 8 (SEQ ID NO:16); residues 1-28, 2-29, 3-30, . . . , 107-134 of the hLy6-BIG polypeptide of Table 9 (SEQ ID NO:18); residues 1-28, 2-29, 3-30, . . . , 34-61 of the hLy6-BIG polypeptide of Table 10 (SEQ ID NO:20); residues 1-28, 2-29, 3-30, . . . , 32-59 of the hLy6-BIG polypeptide of Table 11 (SEQ ID NO:22); residues 1-28, 2-29, 3-30 of the hLy6-BIG polypeptide of Table 12 (SEQ ID NO:24); residues 1-28, 2-29, 3-30, . . . , 32-59 of the hLy6-BIG polypeptide of Table 13 (SEQ ID NO:26); residues 1-28, 2-29, 3-30, . . . , 181-208 of the hLy6-BIG polypeptide of Table 14 (SEQ ID NO:28); residues 1-28, 2-29, 3-30, . . . , 98-125 of the hLy6-BIG polypeptide of Table 15 (SEQ ID NO:30); residues 1-28, 2-29, 3-30, . . . , 93-120 of the hLy6-BIG polypeptide of Table 16 (SEQ ID NO:32); residues 1-28, 2-29, 3-30, . . . , 124-151 of the hLy6-BIG polypeptide of Table 17 (SEQ ID NO:34); residues 1-28, 2-29, 3-30, . . . , 316-343 of the hLy6-BIG polypeptide of Table 18 (SEQ ID NO:36); residues 1-28, 2-29, 3-30, . . . , 114-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 29 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, or 315 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 29 amino acid long fragments including amino acid residues 1-29, 2-30, 3-31, . . . , 106-134 of the hLy6-BIG polypeptide of Table 1 (SEQ ID NO:2); residues 1-29, 2-30, 3-31, . . . , 86-114 of the hLy6-BIG polypeptide of Table 2 (SEQ ID NO:4); residues 1-29, 2-30, 3-31, . . . , 51-79 of the hLy6-BIG polypeptide of Table 3 (SEQ ID NO:6); residues 1-29, 2-30, 3-31, . . . , 99-127 of the hLy6-BIG polypeptide of Table 4 (SEQ ID NO:8); residues 1-29, 2-30, 3-31, . . . , 31-59 of the hLy6-BIG polypeptide of Table 5 (SEQ ID NO:10); residues 1-29, 2-30, 3-31, . . . , 35-63 of the hLy6-BIG polypeptide of Table 6 (SEQ ID NO:12); residues 1-29, 2-30, 3-31, . . . , 34-62 of the hLy6-BIG polypeptide of Table 7 (SEQ ID NO:14); residues 1-29, 2-30, 3-31, . . . , 106-134 of the hLy6-BIG polypeptide of Table 8 (SEQ ID NO:16); residues 1-29, 2-30, 3-31, . . . , 106-134 of the hLy6-BIG polypeptide of Table 9 (SEQ ID NO:18); residues 1-29, 2-30, 3-31, . . . , 33-61 of the hLy6-BIG polypeptide of Table 10 (SEQ ID NO:20); residues 1-29, 2-30, 3-31, . . . , 31-59 of the hLy6-BIG polypeptide of Table 11 (SEQ ID NO:22); residues 1-29, 2-30 of the hLy6-BIG polypeptide of Table 12 (SEQ ID NO:24); residues 1-29, 2-30, 3-31, . . . , 31-59 of the hLy6-BIG polypeptide of Table 13 (SEQ ID NO:26); residues 1-29, 2-30, 3-31, . . . , 180-208 of the hLy6-BIG polypeptide of Table 14 (SEQ ID NO:28); residues 1-29, 2-30, 3-31, . . . , 97-125 of the hLy6-BIG polypeptide of Table 15 (SEQ ID NO:30); residues 1-29, 2-30, 3-31, . . . , 92-120 of the hLy6-BIG polypeptide of Table 16 (SEQ ID NO:32); residues 1-29, 2-30, 3-31, . . . , 123-151 of the hLy6-BIG polypeptide of Table 17 (SEQ ID NO:34); residues 1-29, 2-30, 3-31, . . . , 315-343 of the hLy6-BIG polypeptide of Table 18 (SEQ ID NO:36); residues 1-29, 2-30, 3-31, . . . , 113-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 30 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, or 314 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 30 amino acid long fragments including amino acid residues 1-30, 2-31, 3-32, . . . , 105-134 of the hLy6-BIG polypeptide of Table 1 (SEQ ID NO:2); residues 1-30, 2-31, 3-32, . . . , 85-114 of the hLy6-BIG polypeptide of Table 2 (SEQ ID NO:4); residues 1-30, 2-31, 3-32, . . . , 50-79 of the hLy6-BIG polypeptide of Table 3 (SEQ ID NO:6); residues 1-30, 2-31, 3-32, . . . , 98-127 of the hLy6-BIG polypeptide of Table 4 (SEQ ID NO:8); residues 1-30, 2-31, 3-32, . . . , 30-59 of the hLy6-BIG polypeptide of Table 5 (SEQ ID NO:10); residues 1-30, 2-31, 3-32, . . . , 34-63 of the hLy6-BIG polypeptide of Table 6 (SEQ ID NO:12); residues 1-30, 2-31, 3-32, . . . , 33-62 of the hLy6-BIG polypeptide of Table 7 (SEQ ID NO:14); residues 1-30, 2-31, 3-32, . . . , 105-134 of the hLy6-BIG polypeptide of Table 8 (SEQ ID NO:16); residues 1-30, 2-31, 3-32, . . . , 105-134 of the hLy6-BIG polypeptide of Table 9 (SEQ ID NO:18); residues 1-30, 2-31, 3-32, . . . , 32-61 of the hLy6-BIG polypeptide of Table 10 (SEQ ID NO:20); residues 1-30, 2-31, 3-32, . . . , 30-59 of the hLy6-BIG polypeptide of Table 11 (SEQ ID NO:22); residues 1-30, 2-31, 3-32, . . . , 30-59 of the hLy6-BIG polypeptide of Table 13 (SEQ ID NO:26); residues 1-30, 2-31, 3-32, . . . , 179-208 of the hLy6-BIG polypeptide of Table 14 (SEQ ID NO:28); residues 1-30, 2-31, 3-32, . . . , 96-125 of the hLy6-BIG polypeptide of Table 15 (SEQ ID NO:30); residues 1-30, 2-31, 3-32, . . . , 91-120 of the hLy6-BIG polypeptide of Table 16 (SEQ ID NO:32); residues 1-30, 2-31, 3-32, . . . , 122-151 of the hLy6-BIG polypeptide of Table 17 (SEQ ID NO:34); residues 1-30, 2-31, 3-32, . . . , 314-343 of the hLy6-BIG polypeptide of Table 18 (SEQ ID NO:36); residues 1-30, 2-31, 3-32, . . . , 112-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 31 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, or 313 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 31 amino acid long fragments including amino acid residues 1-31, 2-32, 3-33, . . . , 104-134 of the hLy6-BIG polypeptide of Table 1 (SEQ ID NO:2); residues 1-31, 2-32, 3-33, . . . , 84-114 of the hLy6-BIG polypeptide of Table 2 (SEQ ID NO:4); residues 1-31, 2-32, 3-33, . . . , 49-79 of the hLy6-BIG polypeptide of Table 3 (SEQ ID NO:6); residues 1-31, 2-32, 3-33, . . . , 97-127 of the hLy6-BIG polypeptide of Table 4 (SEQ ID NO:8); residues 1-31, 2-32, 3-33, . . . , 29-59 of the hLy6-BIG polypeptide of Table 5 (SEQ ID NO:10); residues 1-31, 2-32, 3-33, . . . , 33-63 of the hLy6-BIG polypeptide of Table 6 (SEQ ID NO:12); residues 1-31, 2-32, 3-33, . . . , 32-62 of the hLy6-BIG polypeptide of Table 7 (SEQ ID NO:14); residues 1-31, 2-32, 3-33, . . . , 104-134 of the hLy6-BIG polypeptide of Table 8 (SEQ ID NO:16); residues 1-31, 2-32, 3-33, . . . , 104-134 of the hLy6-BIG polypeptide of Table 9 (SEQ ID NO:18); residues 1-31, 2-32, 3-33, . . . , 31-61 of the hLy6-BIG polypeptide of Table 10 (SEQ ID NO:20); residues 1-31, 2-32, 3-33, . . . , 29-59 of the hLy6-BIG polypeptide of Table 11 (SEQ ID NO:22); residues 1-31, 2-32, 3-33, . . . , 29-59 of the hLy6-BIG polypeptide of Table 13 (SEQ ID NO:26); residues 1-31, 2-32, 3-33, . . . , 178-208 of the hLy6-BIG polypeptide of Table 14 (SEQ ID NO:28); residues 1-31, 2-32, 3-33, . . . , 95-125 of the hLy6-BIG polypeptide of Table 15 (SEQ ID NO:30); residues 1-31, 2-32, 3-33, . . . , 90-120 of the hLy6-BIG polypeptide of Table 16 (SEQ ID NO:32); residues 1-31, 2-32, 3-33, . . . , 121-151 of the hLy6-BIG polypeptide of Table 17 (SEQ ID NO:34); residues 1-31, 2-32, 3-33, . . . , 313-343 of the hLy6-BIG polypeptide of Table 18 (SEQ ID NO:36); residues 1-31, 2-32, 3-33, . . . , 111-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 32 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, or 312 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 32 amino acid long fragments including amino acid residues 1-32, 2-33, 3-34, . . . , 103-134 of the hLy6-BIG polypeptide of Table 1 (SEQ ID NO:2); residues 1-32, 2-33, 3-34, . . . , 83-114 of the 111,376-BIG polypeptide of Table 2 (SEQ ID NO:4); residues 1-32, 2-33, 3-34, . . . , 48-79 of the hLy6-BIG polypeptide of Table 3 (SEQ ID NO:6); residues 1-32, 2-33, 3-34, . . . , 96-127 of the hLy6-BIG polypeptide of Table 4 (SEQ ID NO:8); residues 1-32, 2-33, 3-34, . . . . , 28-59 of the hLy6-BIG polypeptide of Table 5 (SEQ ID NO:10); residues 1-32, 2-33, 3-34, . . . , 32-63 of the hLy6-BIG polypeptide of Table 6 (SEQ ID NO:12); residues 1-32, 2-33, 3-34, . . . , 31-62 of the hLy6-BIG polypeptide of Table 7 (SEQ ID NO:14); residues 1-32, 2-33, 3-34, . . . , 103-134 of the hLy6-BIG polypeptide of Table 8 (SEQ ID NO:16); residues 1-32, 2-33, 3-34, . . . , 103-134 of the hLy6-BIG polypeptide of Table 9 (SEQ ID NO:18); residues 1-32, 2-33, 3-34, . . . , 30-61 of the hLy6-BIG polypeptide of Table 10 (SEQ ID NO:20); residues 1-32, 2-33, 3-34, . . . , 28-59 of the hLy6-BIG polypeptide of Table 11 (SEQ ID NO:22); residues 1-32, 2-33, 3-34, . . . , 28-59 of the hLy6-BIG polypeptide of Table 13 (SEQ ID NO:26); residues 1-32, 2-33, 3-34, . . . , 177-208 of the hLy6-BIG polypeptide of Table 14 (SEQ ID NO:28); residues 1-32, 2-33, 3-34, . . . , 94-125 of the hLy6-BIG polypeptide of Table 15 (SEQ ID NO:30); residues 1-32, 2-33, 3-34, . . . , 89-120 of the hLy6-BIG polypeptide of Table 16 (SEQ ID NO:32); residues 1-32, 2-33, 3-34, . . . , 120-151 of the hLy6-BIG polypeptide of Table 17 (SEQ ID NO:34); residues 1-32, 2-33, 3-34, . . . , 312-343 of the hLy6-BIG polypeptide of Table 18 (SEQ ID NO:36); residues 1-32, 2-33, 3-34, . . . , 110-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 33 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, or 311 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 33 amino acid long fragments including amino acid residues 1-33, 2-34, 3-35, . . . , 102-134 of the hLy6-BIG polypeptide of Table 1 (SEQ ID NO:2); residues 1-33, 2-34, 3-35, . . . , 82-114 of the hLy6-BIG polypeptide of Table 2 (SEQ ID NO:4); residues 1-33, 2-34, 3-35, . . . , 47-79 of the hLy6-BIG polypeptide of Table 3 (SEQ ID NO:6); residues 1-33, 2-34, 3-35, . . . , 95-127 of the hLy6-BIG polypeptide of Table 4 (SEQ ID NO:8); residues 1-33, 2-34, 3-35, . . . , 27-59 of the hLy6-BIG polypeptide of Table 5 (SEQ ID NO:10); residues 1-33, 2-34, 3-35, . . . , 31-63 of the hLy6-BIG polypeptide of Table 6 (SEQ ID NO:12); residues 1-33, 2-34, 3-35, . . . , 30-62 of the hLy6-BIG polypeptide of Table 7 (SEQ ID NO:14); residues 1-33, 2-34, 3-35, . . . , 102-134 of the hLy6-BIG polypeptide of Table 8 (SEQ ID NO:16); residues 1-33, 2-34, 3-35, . . . , 102-134 of the hLy6-BIG polypeptide of Table 9 (SEQ ID NO:18); residues 1-33, 2-34, 3-35, . . . , 29-61 of the hLy6-BIG polypeptide of Table 10 (SEQ ID NO:20); residues 1-33, 2-34, 3-35, . . . , 27-59 of the hLy6-BIG polypeptide of Table 11 (SEQ ID NO:22); residues 1-33, 2-34, 3-35, . . . , 27-59 of the hLy6-BIG polypeptide of Table 13 (SEQ ID NO:26); residues 1-33, 2-34, 3-35, . . . , 176-208 of the hLy6-BIG polypeptide of Table 14 (SEQ ID NO:28); residues 1-33, 2-34, 3-35, . . . , 93-125 of the hLy6-BIG polypeptide of Table 15 (SEQ ID NO:30); residues 1-33, 2-34, 3-35, . . . , 88-120 of the hLy6-BIG polypeptide of Table 16 (SEQ ID NO:32); residues 1-33, 2-34, 3-35, . . . , 119-151 of the hLy6-BIG polypeptide of Table 17 (SEQ ID NO:34); residues 1-33, 2-34, 3-35, . . . , 311-343 of the hLy6-BIG polypeptide of Table 18 (SEQ ID NO:36); residues 1-33, 2-34, 3-35, . . . , 109-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 34 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, or 310 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 34 amino acid long fragments including amino acid residues 1-34, 2-35, 3-36, . . . , 101-134 of the hLy6-BIG polypeptide of Table 1 (SEQ ID NO:2); residues 1-34, 2-35, 3-36, . . . , 81-114 of the hLy6-BIG polypeptide of Table 2 (SEQ ID NO:4); residues 1-34, 2-35, 3-36, . . . , 46-79 of the hLy6-BIG polypeptide of Table 3 (SEQ ID NO:6); residues 1-34, 2-35, 3-36, . . . , 94-127 of the hLy6-BIG polypeptide of Table 4 (SEQ ID NO:8); residues 1-34, 2-35, 3-36, . . . , 26-59 of the hLy6-BIG polypeptide of Table 5 (SEQ ID NO:10); residues 1-34, 2-35, 3-36, . . . , 30-63 of the hLy6-BIG polypeptide of Table 6 (SEQ ID NO:12); residues 1-34, 2-35, 3-36, . . . , 29-62 of the hLy6-BIG polypeptide of Table 7 (SEQ ID NO:14); residues 1-34, 2-35, 3-36, . . . , 101-134 of the hLy6-BIG polypeptide of Table 8 (SEQ ID NO:16); residues 1-34, 2-35, 3-36, . . . , 101-134 of the hLy6-BIG polypeptide of Table 9 (SEQ ID NO:18); residues 1-34, 2-35, 3-36, . . . , 28-61 of the hLy6-BIG polypeptide of Table 10 (SEQ ID NO:20); residues 1-34, 2-35, 3-36, . . . , 26-59 of the hLy6-BIG polypeptide of Table 11 (SEQ ID NO:22); residues 1-34, 2-35, 3-36, . . . , 26-59 of the hLy6-BIG polypeptide of Table 13 (SEQ ID NO:26); residues 1-34, 2-35, 3-36, . . . , 175-208 of the hLy6-BIG polypeptide of Table 14 (SEQ ID NO:28); residues 1-34, 2-35, 3-36, . . . , 92-125 of the hLy6-BIG polypeptide of Table 15 (SEQ ID NO:30); residues 1-34, 2-35, 3-36, . . . , 87-120 of the hLy6-BIG polypeptide of Table 16 (SEQ ID NO:32); residues 1-34, 2-35, 3-36, . . . , 118-151 of the hLy6-BIG polypeptide of Table 17 (SEQ ID NO:34); residues 1-34, 2-35, 3-36, . . . , 310-343 of the hLy6-BIG polypeptide of Table 18 (SEQ ID NO:36); residues 1-34, 2-35, 3-36, . . . , 108-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may be at least 35 amino acids in length, and may begin at amino acid residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, or 309 of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). Thus, polypeptides of the invention may comprise or consist of 35 amino acid long fragments including amino acid residues 1-34, 2-35, 3-36, . . . , 100-134 of the hLy6-BIG polypeptide of Table 1 (SEQ ID NO:2); residues 1-34, 2-35, 3-36, . . . , 80-114 of the hLy6-BIG polypeptide of Table 2 (SEQ ID NO:4); residues 1-34, 2-35, 3-36, . . . , 45-79 of the hLy6-BIG polypeptide of Table 3 (SEQ ID NO:6); residues 1-34, 2-35, 3-36, . . . , 93-127 of the hLy6-BIG polypeptide of Table 4 (SEQ ID NO:8); residues 1-34, 2-35, 3-36, . . . , 25-59 of the hLy6-BIG polypeptide of Table 5 (SEQ ID NO:10); residues 1-34, 2-35, 3-36, . . . , 29-63 of the hLy6-BIG polypeptide of Table 6 (SEQ ID NO:12); residues 1-34, 2-35, 3-36, . . . , 28-62 of the hLy6-BIG polypeptide of Table 7 (SEQ ID NO:14); residues 1-34, 2-35, 3-36, . . . , 100-134 of the hLy6-BIG polypeptide of Table 8 (SEQ ID NO:16); residues 1-34, 2-35, 3-36, . . . , 100-134 of the hLy6-BIG polypeptide of Table 9 (SEQ ID NO:18); residues 1-34, 2-35, 3-36, . . . , 27-61 of the hLy6-BIG polypeptide of Table 10 (SEQ ID NO:20); residues 1-34, 2-35, 3-36, . . . , 25-59 of the hLy6-BIG polypeptide of Table 11 (SEQ ID NO:22); residues 1-34, 2-35, 3-36, . . . , 25-59 of the hLy6-BIG polypeptide of Table 13 (SEQ ID NO:26); residues 1-34, 2-35, 3-36, . . . , 174-208 of the hLy6-BIG polypeptide of Table 14 (SEQ ID NO:28); residues 1-34, 2-35, 3-36, . . . , 91-125 of the hLy6-BIG polypeptide of Table 15 (SEQ ID NO:30); residues 1-34, 2-35, 3-36, . . . , 86-120 of the hLy6-BIG polypeptide of Table 16 (SEQ ID NO:32); residues 1-34, 2-35, 3-36, . . . , 117-151 of the hLy6-BIG polypeptide of Table 17 (SEQ ID NO:34); residues 1-34, 2-35, 3-36, . . . , 309-343 of the hLy6-BIG polypeptide of Table 18 (SEQ ID NO:36); residues 1-34, 2-35, 3-36, . . . , 107-141 of the hLy6-BIG polypeptide of Table 19 (SEQ ID NO:38). An antibody of the invention may specifically bind one of the above fragments, or more than one fragments which overlap. Thus, the invention also includes antibodies which bind one or more polypeptides of the invention as well as methods for making such antibodies and compositions comprising such antibodies. The invention also includes polynucleotides encoding such polypeptides and such antibodies.

Polypeptide fragments of the invention may contain a continuous series of deleted residues from the amino (N)- or the carboxyl (C)-terminus, or both. For example, any number of amino acids, ranging from 1 to 338, can be deleted from the N-terminus. Polypeptides of the invention may comprise or consist of fragments containing a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the N-terminus of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). The invention also includes polynucleotides encoding such polypeptides.

Additionally, N-terminal deletion fragments of the invention may contain a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, or 338 amino acids from the N-terminus of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). The invention also includes polynucleotides encoding such polypeptides.

As another example, any number of amino acids, ranging from 1 to 338, can be deleted from the C-terminus. Polypeptides of the invention may comprise or consist of fragments containing a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). The invention also includes polynucleotides encoding such polypeptides.

Additionally, C-terminal deletion fragments of the invention may contain a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, or 338 amino acids from the C-terminus of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or the hLy6-BIG proteins encoded by the deposited clones). The invention also includes polynucleotides encoding such polypeptides.

Furthermore, polypeptides of the invention may comprise or consist of fragments which contain combinations of N- and C-terminal deletions such as the N-terminal and C-terminal deletions described above. Combined N- and C-terminal deletion fragments of the invention may contain a deletion of Ito 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230 amino acids from the N-terminus and may also contain a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230 amino acids from the C-terminus. The invention also includes polynucleotides encoding such polypeptides.

Thus, exemplary polypeptides of the invention include polypeptides which comprise or consist of amino acids 23 to 130, 55 to 121, 73 to 111, 5 to 93, 110 to 128, 118 to 130, 45 to 120, 31 to 121, 41 to 93, 35 to 98 of the hLy6-BIG protein in Table 1, 8, 9, 17, or 19. Additional exemplary of polypeptides of the invention include polypeptides which comprise or consist of amino acids 4-79, 18-111, 12-53, 57-103, 78-109, 44-100 of the hLy6-BIG protein in Table 2, 4, 15, or 16. Other exemplary of polypeptides of the invention include polypeptides which comprise or consist of amino acids 6-52, 20-36, 12-55, 40-50 of the hLy6-BIG protein in Table 3, 5, 6, 7, 10, 11, or 13. Other exemplary of polypeptides of the invention include polypeptides which comprise or consist of amino acids 6-23, 10-18, 18-26, 9-27 of the hLy6-BIG protein in Table 12. Other exemplary of polypeptides of the invention include polypeptides which comprise or consist of amino acids 4-200, 60-120, 87-95, 141-199, 10-23, 32-145 of the hLy6-BIG protein in Table 14. Other exemplary of polypeptides of the invention include polypeptides which comprise or consist of amino acids 10-321, 15-38, 44-118, 12-99, 320-338 of the hLy6-BIG protein in Table 18. The invention further includes nucleic acid molecules which encodes these polypeptides of the invention, as well as other polypeptides described herein, and host cells which contain such nucleic acid molecules. The invention further includes methods for making polypeptides of the invention (e.g., methods for producing polypeptides using nucleic acid molecules of the invention). In particular embodiments, polypeptides of the invention are provided in (1) isolated, (2) substantially pure, and/or (3) essentially pure forms. The invention further includes compositions and mixtures (e.g., reaction mixtures) which contain one or more polypeptides and/or polynucleotides of the invention. The invention also includes polynucleotides encoding such polypeptides.

Combined N- and C-terminal deletion fragments of the invention may contain combinations of deletions such as a deletion of 1 to 10 (e.g., 1, 2, 3, 4, 5, 6, 7, 8, 9, or 10) amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 10 to 20 (e.g., 11, 12, 13, 14, 15, 16, 17, 18, 19, or 20) amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 20 to 30 (e.g., 20, 21, 22, 23, 24, 25, 26, 27, 28, 29, or 30) amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 30 to 40 (e.g., 30, 31, 32, 33, 34, 35, 36, 37, 38, 39, or 40) amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 40 to 50 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 50 to 60 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus.

Combined N- and C-terminal deletion fragments of the invention may contain combinations of deletions such as a deletion of 60 to 70 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 70 to 80 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 80 to 90 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 90 to 100 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus The invention also includes polynucleotides encoding such polypeptides.

Combined N- and C-terminal deletion fragments of the invention may contain combinations of deletions such as a deletion of 100 to 110 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 110 to 120 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 120 to 130 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 130 to 140 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 140 to 150 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 150 to 160 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 160 to 170 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 170 to 180 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 180 to 190 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 190 to 200 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 200 to 210 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 210 to 220 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus The invention also includes polynucleotides encoding such polypeptides.

Combined N- and C-terminal deletion fragments of the invention may contain combinations of deletions such as a deletion of 220 to 230 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Combined N- and C-terminal deletion fragments of the invention may contain combinations of deletions such as a deletion of 230 to 240 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, or 220 to 230 amino acids from the C-terminus. The invention also includes polynucleotides encoding such polypeptides.

Even if deletion of one or more amino acids from the N- and/or C-terminus of a protein results in modification of loss of one or more biological functions of the protein, other functional activities (e.g., enzymatic activities, antigenic activity, immunogenic activity) may still be retained. For example, the ability of shortened polypeptides to induce and/or bind to antibodies which recognize the complete forms of the polypeptides generally will be retained when less than the majority of the residues of the complete or mature polypeptide are removed from the N- and/or C-terminus. Whether a particular polypeptide lacking N- and/or C-terminal residues of a complete polypeptide retains such immunologic activities can readily be determined by routine methods described herein and otherwise known in the art. It is not unlikely that a fragment with a large number of deleted N- and/or C-terminal amino acid residues may retain some antigenic or immunogenic activities. In fact, peptides composed of as few as six amino acid residues may often evoke an immune response, as discussed below.

Polypeptide fragments of the invention may include unique regions, i.e., stretches of amino acids of the hLy6-BIG proteins of Tables 1-19 (SEQ ID NOS: 2, 4, 6, 8, 10, 12, 14, 16, 18, 20, 22, 24, 26, 28, 30, 32, 34, 36, or 38) that are less than 100% identical to corresponding stretches of amino acids in other proteins such the polypeptides of FIGS. 2 and 12 (SEQ ID NOS:______). Unique regions of each polypeptide (hLy6-BIG protein) of the invention are shown in the alignment in FIGS. 2 and 12, which indicates the identical and non-identical amino acids of the hLy6-BIG proteins of Tables 1-19 (SEQ ID NOS: 2, 4, 6, 8, 10, 12, 14, 16, 18, 20, 22, 24, 26, 28, 30, 32, 34, 36, or 38) (or the hLy6-BIG proteins encoded by a deposited clones) as compared to the related polypeptides. Polypeptide fragments of the invention containing unique regions are useful for generating highly specific antibodies of the invention, as discussed below, and for conferring upon a protein a particular activity, such as an activity described herein. Thus, fragments containing unique regions are preferred antigenic fragments of the invention. Additionally, fragments containing unique regions are also useful for producing fusion proteins such as proteins produced by DNA shuffling, described in more detail below. Using DNA shuffling, fusion proteins are constructed which comprise fragments from one or more hLy6-BIG proteins and which preferably have an activity of a hLy6-BIG protein of any of Tables 1-19 (SEQ ID NOS: 2, 4, 6, 8, 10, 12, 14, 16, 18, 20, 22, 24, 26, 28, 30, 32, 34, 36, or 38) or the hLy6-BIG proteins encoded by a deposited clone.

Other fragments of the invention are fragments characterized by structural or functional attributes of the polypeptides of the invention. See Table 22, and FIGS. 9A-9S and 10A-10G.

Such fragments include amino acid residues that comprise alpha helix and alpha helix forming regions (“alpha regions”), beta sheet and beta sheet forming regions (“beta regions”), turn and turn forming regions (“turn regions”), coil and coil forming regions (“coil regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson Wolf program) of polypeptides of the invention (e.g., hLy6-BIG proteins of Tables 1-19 (SEQ ID NOS: 2, 4, 6, 8, 10, 12, 14, 16, 18, 20, 22, 24, 26, 28, 30, 32, 34, 36, or 38)). Certain preferred regions include, but are not limited to, regions of the aforementioned types identified by analysis of the amino acid sequence depicted in Tables 1-19 (SEQ ID NOS: 2, 4, 6, 8, 10, 12, 14, 16, 18, 20, 22, 24, 26, 28, 30, 32, 34, 36, or 38), such preferred regions include; Garnier Robson predicted alpha regions, beta regions, turn regions, and coil regions; Chou Fasman predicted alpha regions, beta regions, turn regions, and coil regions; Kyte Doolittle predicted hydrophilic and hydrophobic regions; Eisenberg alpha and beta amphipathic regions; Emini surface forming regions; and Jameson Wolf high antigenic index regions, as predicted using the default parameters of these computer programs. These structural or functional attributes can be generated using the various modules and algorithms of the DNA*STAR program set on default parameters. The invention also includes polynucleotides encoding such polypeptides.

Other preferred regions of the invention include those defined in FIGS. 9A-9S and 10A-10G and in Table 22.

Among preferred polypeptide fragments of the invention in this regard are those that comprise regions of the polypeptides that combine several structural features, such as several of the features set out above or below. The invention also includes polynucleotides encoding such polypeptides.

In another embodiment, the polypeptide may comprise or consist of one or more polypeptide fragments (e.g., regions) such as a polypeptide fragment of the invention described herein. For a polypeptide comprising or consisting of the amino acid sequence of two or more fragments (e.g., regions), the fragments (e.g., regions) may be contiguous with one another. In one embodiment, the fragments (e.g., regions) are not contiguous with one another, i.e., they are separated by one or more amino acid residues.

Preferably, the fragments (e.g., regions) align with the corresponding regions of the full length polypeptide such that they are separated by the same number of amino acid residues as separate them in the hLy6-BIG protein (e.g., the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met, (or the hLy6-BIG proteins encoded by the deposited clones).

Polypeptide fragments of the invention may contain antigenic regions (i.e., regions to which an antibody will bind; epitopes) of the polypeptides of the invention. Antigenic regions may be as small as 6 amino acids. Polypeptide fragments of the invention which function as antigenic epitopes may be produced by any conventional means. See, e.g., Houghten, R. A., Proc. Natl. Acad. Sci. USA 82:5131 5135 (1985) further described in U.S. Pat. No. 4,631,211.

As to the selection of fragments bearing an antigenic region, it is well known in that art that relatively short synthetic peptides that mimic part of a protein sequence are routinely capable of eliciting an antiserum that reacts with the partially mimicked protein. See, e.g., Sutcliffe, J. G., Shinnick, T. M., Green, N. and Learner, R. A., Science 219:660 666 (1983).

Polypeptide fragments of the invention capable of eliciting protein reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of fewer than six residues generally are ineffective at inducing antibodies that bind to the mimicked protein; longer, peptides, especially those containing proline residues, usually are effective. Sutcliffe et al., supra, at 661. For instance, 18 of 20 peptides designed according to these guidelines, containing 8 39 residues covering 75% of the sequence of the influenza virus hemagglutinin HA1 polypeptide chain, induced antibodies that reacted with the HA1 protein or intact virus; and 12/12 peptides from a MuLV protein and 18/18 from the rabies glycoprotein induced antibodies that precipitated the respective proteins. Thus, the invention includes polypeptides comprising or consisting of fragments of the full length hLy6-BIG protein (the polypeptides of Tables 1-19 (SEQ ID NOS:2, 4, 6, 8, 10, 12, 14, 16, 18, 20, 22, 24, 26, 28, 30, 32, 34, 36, or 38) with or without the N-terminal Met, (or the hLy6-BIG proteins encoded by the deposited clones) which are at least 6, 10, 12, 14, 18, or 20 amino acids in length and have one or more of the following features: (1) is not extremely hydrophobic, and/or (2) contains one or more proline residues.

Antigenic fragments of the invention, and polypeptides comprising them, are therefore useful to raise antibodies, including monoclonal antibodies, that bind specifically to a polypeptide of the invention. Thus, a high proportion of hybridomas obtained by fusion of spleen cells from donors immunized with an antigen epitope bearing peptide generally secrete antibody that binds the native protein. Sutcliffe et al., supra, at 663. The antibodies raised by antigenic fragments or polypeptides comprising them are useful to detect the polypeptides of the invention, and antibodies to different fragments may be used for tracking the fate of various regions of a protein precursor which undergoes post translational processing. The fragments and anti fragment antibodies may be used in a variety of qualitative or quantitative assays for the mimicked protein, for instance in competition assays since it has been shown that even short peptides (e.g. about 9 amino acids) can bind and displace the larger peptides in immunoprecipitation assays. See, for instance, Wilson et al., Cell 37:767 778 (1984) at 777. The antibodies of the invention also are useful for purification of the polypeptides of the invention, for instance, by adsorption chromatography using methods well known in the art.

Antigenic fragments and polypeptides of the invention designed according to the above guidelines preferably contain a sequence of at least seven, more preferably at least nine and most preferably between about 15 to about 30 amino acids contained within the amino acid sequence of a polypeptide of the invention. However, fragments and polypeptides comprising, or alternatively consisting of, a larger portion such as about 30 to about 50 amino acids, or any length up to and including the entire amino acid sequence of a polypeptide of the invention, also are considered antigenic fragments or polypeptides of the invention and also are useful for inducing antibodies that react with the full length polypeptide. Preferably, the amino acid sequence of the antigenic fragment is selected to provide substantial solubility in aqueous solvents (i.e., the sequence includes relatively hydrophilic residues and highly hydrophobic sequences are preferably avoided); and sequences containing proline residues are particularly preferred.

In the present invention, antigenic fragments preferably contain (comprise or consist of) a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising antigenic fragments are at least 10, 15, 20, 25, 30, 35, 40, 45, 50, 55, 60, 65, 70, 75, 80, 85, 90, 95, or 100 amino acid residues in length. Additional non exclusive preferred antigenic fragments include the fragments disclosed herein, as well as portions thereof. Antigenic fragments are useful, for example, to raise antibodies, including monoclonal antibodies, that specifically bind the epitope. Preferred antigenic fragments include the fragments disclosed herein, as well as any combination of two, three, four, five or more of these fragments. Antigenic fragments can be used as the target molecules in immunoassays. (See, for instance, Wilson et al., Cell 37:767 778 (1984); Sutcliffe et al., Science 219:660 666 (1983)).

Similarly, antigenic fragments can be used, for example, to induce antibodies according to methods well known in the art. (See, for instance, Sutcliffe et al., supra; Wilson et al., supra; Chow et al., Proc. Natl. Acad. Sci. USA 82:910 914; and Bittle et al., J. Gen. Virol. 66:2347 2354 (1985). The polypeptides comprising, or alternatively consisting of, one or more antigenic fragments may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, antigenic fragments comprising as few as 8 to 10 amino acids have been shown to be sufficient to raise antibodies capable of binding to, at the very least, linear epitopes in a denatured polypeptide (e.g., in Western blotting).

Polypeptides of the invention may comprise or consist of variants of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38)) with or without the N-terminal Met, variants of the polypeptides encoded by the deposited clones, and variants of the fragments described above. Variants include polypeptides which are at least 80%, 81%, 82%, 83%, 84%, 85%, 86%, 87%, 88%, 89%, 90%, 91%, 92%, 93%, 94%, 95% 96%, 97%, 98%, or 99% identical to a polypeptide encoded by a deposited clone, or to a hLy6-BIG protein of any of Tables 1-19 (SEQ ID NOS: 2, 4, 6, 8, 10, 12, 14, 16, 18, 20, 22, 24, 26, 28, 30, 32, 34, 36, or 38), or to a fragment described above.

Thus, the invention includes, in part, polypeptides which are at least 80%, 81%, 82%, 83%, 84%, 85%, 86%, 87%, 88%, 89%, 90%, 91%, 92%, 93%, 94%, 95% 96%, 97%, 98%, or 99% identical to (1) a polypeptide encoded by a deposited clone described herein, (2) a hLy6-BIG protein having an amino acid sequence set out in any of Tables 1-19 (SEQ ID NOS: 2, 4, 6, 8, 10, 12, 14, 16, 18, 20, 22, 24, 26, 28, 30, 32, 34, 36, or 38), or (3) to a subportion of one of these hLy6-BIG proteins. The invention further includes nucleic acid molecules which encode these polypeptides, as well as host cells which contain such nucleic acid molecules. The invention also includes compositions and mixtures (e.g., reaction mixtures) which contain one or more polypeptides and/or polynucleotides of the invention.

In many instances, the above described polypeptides, as well as other polypeptides of the invention, will have one or more activity associated with a polypeptide encoded by a deposited clone described herein or a hLy6-BIG protein having an amino acid sequence set out in any of Tables 1-19 (SEQ ID NOS: 2, 4, 6, 8, 10, 12, 14, 16, 18, 20, 22, 24, 26, 28, 30, 32, 34, 36, or 38).

It will be recognized in the art that some amino acid sequences of the polypeptides of the invention can be varied without significant affect on the structure or function of the protein. If such differences in sequence are contemplated, it should be remembered that there may be critical areas on the protein which determine activity. In general, it is possible to replace residues which form the tertiary structure, provided that residues performing a similar structural or enzymatic function are used. In other instances, the type of residue may be completely unimportant if the alteration occurs at a non critical region of the protein.

Thus, the invention includes variants which may show a functional activity. Preferably, the variants demonstrate a functional activity such as antigenicity or an enzymatic activity described above.

The functional activity of polypeptides of the invention can be assayed by various methods. For example, in one embodiment where one is assaying for antigenicity, various immunoassays known in the art can be used, including but not limited to, competitive and non competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In addition, assays described herein and otherwise known in the art may routinely be applied to measure the ability of variants to elicit an enzymatic activity.

Variants include deletions, insertions, inversions, repeats, and substitutions (e.g., conservative substitutions, non-conservative substitutions, type substitutions (for example, substituting one hydrophilic residue for another hydrophilic residue, but not a strongly hydrophilic for a strongly hydrophobic, as a rule), primary shifts, primary transpositions, secondary transpositions, and coordinated replacements).

More than one amino acid (e.g., 2, 3, 4, 5, 6, 7, 8, 9, 10, etc.) can be deleted or inserted or can be substituted with another amino acid as described above (either conservative or nonconservative). Preferably, a single amino acid is substituted with a single amino acid, however, a polypeptide of the invention may contain any number of single amino acid substitutions, as described above and below. The deletion, insertion, or substitution can occur in the full length, mature, or proprotein form of the polypeptide, as well as in the fragments described above.

Variants may contain at least one amino acid substitution, deletion or insertion but not more than 50 (e.g., 15, 18, 20, 30, 35, 40, etc.) amino acid substitutions, deletions or insertions, even more preferably, not more than 40 amino acid substitutions, deletions or insertions, still more preferably, not more than 30 amino acid substitutions, deletions or insertions, and still even more preferably, not more than 20 amino acid substitutions, deletions or insertions. Of course, in order of increasing preference, it is preferable for a variant to contain at least one, but not more than 10, 9, 8, 7, 6, 5, 4, 3, 2 or 1 amino acid substitutions, deletions or insertions. In specific embodiments, the number of additions, substitutions, and/or deletions in the polypeptide (e.g., the full length form and/or fragments described herein), is 1 5, 5 10, 5 25, 5 50, 10 50 or 50 150. Conservative amino acid substitutions are preferable in some embodiments.

Of course, the number of amino acid substitutions a skilled artisan would make depends on many factors, including those described above and below. Preferred amino acid substitutions are described herein.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Of additional special interest are also substitutions of charged amino acids with another charged amino acid or with neutral amino acids. This may result in proteins with improved characteristics such as less aggregation. Prevention of aggregation is highly desirable. Aggregation of proteins can result in a reduced activity.

Guidance concerning how to make phenotypically silent amino acid substitutions is provided in Bowie, J. U. et al., wherein the authors indicate that there are two main strategies for studying the tolerance of an amino acid sequence to change. Bowie, J. U. et al., “Deciphering the Message in Protein Sequences: Tolerance to Amino Acid Substitutions,” Science 247:1306 1310 (1990)

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine scanning mutagenesis (introduction of single alanine mutations at every residue in the molecule) can be used. (Cunningham and Wells, Science 244:1081 1085 (1989).) The resulting mutant molecules can then be tested for functional activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved.

Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small sized amino acids Ala, Ser, Thr, Met, and Gly.

Thus, residues important for a particular functional activity (e.g., enzymatic, antigenic or immunogenic activity) may be identified by mutagenesis strategies designed to locally perturb the protein. In alanine scanning mutagenesis, all non-alanine residues of the protein (or of a region of the protein suspected to contain the binding site are replaced, one-by-one, with alanine, yielding a collection of single substitution mutants. Alanine is used because (1) it is the most common amino acid residue in proteins, (2) it has a small side chain, and therefore is not likely to sterically hinder other residues, and (3) its side chain does not form H-bonds, but is not especially hydrophobic. Cunningham and Wells (1989) conducted an Ala scanning mutagenesis study of residues 2-19, 54-74, and 167-191 in hGH. A total of 62 Ala mutations were produced. Of these, fourteen mutants destabilized the protein, eleven mutants seemingly enhanced activity. Of the remaining 37 mutants, only four impaired binding by 10-fold or more, and only nine by 5-fold or more. See generally WO90/04788.

For other uses of Ala-scan mutagenesis, see Yu et al (1995) (complete scan of a single disulfide derivative of the 58-residue protein BPTI); Allen et al (1987) (Ala-scan of residues 52-61 of hen egg white lysozyme); Ruf et al (1994) (Ala-scan of residues other than Gly, Pro and Cys; multiple Ala mutants examined first, then single Ala mutants); Williams et al (1995) (Ala-scan in insulin receptor of (1) charged amino acids, (2) aromatic residues, and (3) residues adjacent to (1) or (2), other than prolines, cysteines, or potential N-linked glycosylation sites); Kelly et al (1993) (Ala-scan of antibody CDR). Ala-scanning mutagenesis may be applied to all residues of a protein, or to residues selected on some rational basis, such as amino acid type (e.g., charged and aromatic residues), degree of variability in a homologous protein family, or relevance to function as shown by homologue-scanning mutagenesis.

Preferably, further mutations (especially non-conservative mutations) are made at sites where an alanine substitution does not lead to a decrease in an activity of interest of more than 20-fold, more preferably, of more than 10-fold, even more preferably, of more than 5-fold, still more preferably, of more than 2-fold. Most preferably, mutations are made at sites at which an alanine substitution improves activity.

Preferably, when multiple mutations are made, the expected (additive) effect of the mutations is one which does not lead to a decrease in activity of more than 10-fold, more preferably, of more than 5 fold, still more preferably, of more than two fold. Most preferably, the expected effect is to improve activity. The expected effect of a conservative substitution is the effect of that mutation as a single substitution if known, or otherwise neutral. The expected effect of a non-conservative substitution is the effect of that mutation as a single substitution if known, or otherwise the effect of a single substitution of a different residue of the same exchange group as the actual replacement residue, if known, or otherwise the effect of a single Ala substitution.

Another approach is homologue-scanning mutagenesis. This involves identifying a homologue which can be distinguished in an activity assay from the protein of interest, and screening mutants in which a segment of the protein of interest is replaced by corresponding segments of the homologue (or vice versa). Proteins that may be used as homologues include previously identified hLy6-BIG proteins such as those in FIGS. 2 and 12 or otherwise known in the art. If the replacement alters the activity of the modified protein, the segment in question presumably contributes to the observed difference in activity between the protein of interest and the homologous protein, and comparison of the interchanged segments helps to explain the character of the binding site involved in that activity. For example, segments of prolactin, which does not bind the GH receptor, have been used to replace segments of growth hormone, which does. If a substitution disrupts GH binding, it implies that the replaced segment was part of the GH receptor binding site, and one may then focus on how the replaced and replacing segments differ. See WO 90/04788.

If a residue is determined to be a part of the enzymatic or binding site, one may prepare all possible single substitution mutants of that site.

It is possible to incorporate two or more tolerable mutations into a protein. Generally speaking, as a first approximation, it is reasonable to assume that the effect of two or more mutations will be additive in nature. See Wells (1990); Sandberg and Terwilliger (1993); Gregoret and Sauer (1993); Schreiber and Fersht (1995); et al (1993); Lowman et al (1991); Lin et al (1994); Venkatachalam et al (1994); Akasako et al (1995); Behravan et al (1991); Lin et al (1994); Zuckermann et al (1992).

Non-additive effects are more likely to occur between residues that are in Van der Waals contact with each other. See Sandberg and Terwilliger (1993). According to Schreiber and Fersht (1995), non-additive effects are more likely to occur between residues less than 7 Angstrom apart (10 Angstrom in the case of charged residues). The effect of a second mutation on a first one may be synergistic, additive, partially additive, neutral, antagonistic, or suppressive. Long range but low magnitude departures from additivity may occur reasonably often, see LiCata and Ackers (1995), but do not significantly impair the value of multiple mutation in protein engineering.

Gregoret et al (1993) assumed that, under selective conditions, the frequency of occurrence of a mutation in an active mutant was an indication of whether the mutant conferred resistance, and found that an additive model (multiplying the mutational frequencies of a pair of single Ala substitution mutants) was about 90% effective in predicting the activity class of a binomial (multiple Ala substitution) mutant.

The most common reason for combining mutations is to benefit from their additive or synergistic effect in combination. For example, if a mutation has both favorable and unfavorable activities, it may be possible to combine it with a second mutation that neutralizes the unfavorable activity of the first mutation.

One use of multiple mutation is to achieve, by combining mutations which individually have a small but favorable effect on activity, a mutant with a more substantial improvement in activity. It is not necessary that the mutations be strictly additive; it is sufficient that they be at least partially additive for the combination to be advantageous. See Blacklow et al (1991) (improved catalytic effectiveness of triosephosphate isomerase); Akasako et al (1995) (multiple thermostabilizing mutations in ribonuclease HI); Lowman et al (1991) (HGH-receptor binding properties of human placental lactogen improved about 500-fold by five simultaneous, mutations, with “reasonably additive” effects); Lowman and Wells (1993) (HGH-receptor binding properties of HGH improved about 400-fold by combination of 15 substitutions. Sandberg and Terwilliger (1993), reported that there was only a weak correlation between changes in DNA binding protein stability and changes in DNA binding affinity, and hence that it was possible to combine mutations so as to selectively change one property without changing the other.

Watanabe et al (1994) suggests that increasing the number of proline residues, especially at second sites of beta turns and N-caps of alpha helices, increases the thermostability of the protein in an additive manner.

Gloss et al (1992) converted all cysteines of a protein to alanine. They point out that this cysteine-free mutant provides a platform onto which uniquely placed cysteine residues may be engineered, thereby allowing the introduction of unnatural amino acids through exploitation of the unique reactivity of the thiol group.

The interactivity of two residues is generally determined by preparing both single substitution mutants as well as a double substitution mutant, and determining whether the effects are additive or not. Therefore, if single Ala substitutions have been shown to favorably or unfavorably affect activity, one may prepare a double Ala mutant and compare its activity to that of the single substitution mutants. While it is certainly possible that two mutations which, by themselves, do not affect activity, may do so when combined, this is unlikely, especially if the sites are not close together.

One could prepare all possible double Ala mutants, which would mean preparing N(N-1) mutants, where N was the number of non-Ala residues in the protein. In general, it is preferable to limit the double substitution studies to sites known to favorably affect the activity. Possibly, one would also consider sites which were strongly unfavorable (to look for antagonistic interactions).

Another approach is binomial Ala-scanning mutagenesis. Here, one constructs a library in which, at each position of interest of a given protein molecule, the residue is randomly either the native residue, or Ala. See Gregoret and Sauer (1993). It is feasible to screen a library of 1010 mutants, so the combined effects of up to 30 different Ala substitutions (about 227 to about 1010) can be studied in one experiment. It should be noted that the Ala:non-Ala ratio at each position may be, but need not be equal.

If the protein is too large for all sites of interest to be sampled by binomial Ala-scanning mutagenesis in a single experiment, one may divide the protein into segments and subject each segment in turn to such mutagenesis, and then, as a cross-check, similarly mutate one residue from each segment.

Even when mutations are not additive in effect, this is may be desirable. Green and Shortie, (1993) reported that mutations which individually reduced stability, when not additive in their effects, were almost exclusively sub-additive, i.e., the reduction in stability was less than that expected by summing the individual destabilizations. This is credited to an overlap of the “spheres of perturbation” surrounding the two mutations. Ballinger et al (1995) reported that a combination subtilisin BPN′ mutant had a larger than additive shift in specificity toward dibasic substrates, which is a desirable change.

Certain multiple mutations are worthy of special comment, as follows.

Primary shifts: In a primary shift the residue at position n becomes the replacement amino acid at position n+s, or vice versa. For example, instead of Cys at 30, one might have Cys at 31. The result is a mere displacement, rather than a loss, of the amino acid in question. In a primary shift, s (the shift distance) is most often equal to one, but may be two, three or more. The greater the value of s, the more the shift resembles an ordinary double mutation.

Primary transpositions: In a primary transposition, the residues at positions n and n+s in the primary amino acid sequence are swapped. Such swaps are less likely to perturb the protein than the individual replacements, examined singly, might suggests. A primary transposition is, in effect, a combination of two complementary shifts.

Secondary Transposition: Here, two amino acids which interact as a result of the folding of the protein are swapped. A classic example would be members of a salt bridge. If there is an Asp in one segment forming a salt bridge with a Lys in another segment, the Asp and Lys can be swapped, and a salt bridge can still form.

Coordinated Replacement: Here, replacement of residue x is coordinated with replacement of residue y. Thus, replacement of one Cys may be coordinated with replacement of a second Cys with which it otherwise forms a disulfide bond, and if one amino acid of a pair forming a salt bridge is replaced by an uncharged a.a., the other may likewise be replaced.

Techniques of detecting coordinated amino acid changes in families of homologous proteins are discussed in Altschuh et al (1988).

Primary shifts, primary transpositions, secondary transpositions and coordinated replacements are more likely to be tolerated than other multiple mutations involving the same individual amino acid changes.

Examples of production of amino acid substitutions in proteins which can be used for obtaining variants of the present invention include any known method steps, such as presented in U.S. Pat. RE 33,653, U.S. Pat. Nos. 4,959,314, 4,588,585 and 4,737,462, to Mark et al; U.S. Pat. No. 5,116,943 to Koths et al, U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al).

Polypeptides of the invention may be altered by being subjected to random mutagenesis by error-prone PCR, random nucleotide insertion or other methods prior to recombination. Polypeptides of the invention may be produced by DNA shuffling, gene-shuffling, motif-shuffling, exon-shuffling, and/or codon-shuffling (collectively referred to as “DNA shuffling”). DNA shuffling involves the assembly of two or more DNA segments by homologous or site-specific recombination to generate variation in the polynucleotide sequence. DNA shuffling may be employed to modulate the activities of polypeptides of the invention, such methods can be used to generate polypeptides with altered activity. See, generally, U.S. Pat. Nos. 5,605,793; 5,811,238; 5,830,721; 5,834,252; 5,837,458; and 6,444,468; and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). Thus, one or more components, motifs, sections, parts, domains, fragments, etc., of a polypeptide of the invention may be joined to one or more components, motifs, sections, parts, domains, fragments, etc. of one or more heterologous molecules, preferably the hLy6-BIG proteins in Tables 1-19 (SEQ ID NOS: 2, 4, 6, 8, 10, 12, 14, 16, 18, 20, 22, 24, 26, 28, 30, 32, 34, 36, or 38).

Polypeptides comprising fragments, mutants, variants, or full length polypeptides of the invention may be “free standing,” or comprised within a larger polypeptide of which the fragment, mutant, variant, or full length polypeptide forms a part or region.

Thus, the polypeptides may include one or more additional amino acids and/or one or more heterologous sequences such as those described herein. For instance, a methionine residue may be added to the N-terminus of the polypeptide (e.g., polypeptides comprising or consisting of fragments, variants, etc.) to allow for recombinant expression. Also, a sequence of additional amino acids, particularly charged amino acids, may be added to the N terminus of the polypeptide to improve stability and persistence, in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP A 0 464 533 (Canadian counterpart 2045869) discloses fusion proteins comprising various portions of constant region of immunoglobin molecules together with another protein or part thereof. For some uses it would be desirable to be able to remove the Fc part after the fusion protein has been expressed, detected and purified in the advantageous manner described. This is the case when Fc portion proves to be a hindrance, for example when the fusion protein is to be used as an immunogen for raising antibodies. In drug discovery, for example, human proteins, such as hIL5 receptor, have been fused with Fc portions for the purpose of high throughput screening assays to identify antagonists of hIL 5. See, D. Bennett et al., Journal of Molecular Recognition, Vol. 8:52 58 (1995) and K. Johanson et al., The Journal of Biological Chemistry, Vol. 270, No. 16:9459 9471 (1995).

Thus, the polypeptides may be in the form of the secreted protein, including a mature form, or may be a part of a larger protein, such as a fusion protein. It is often advantageous to include an additional amino acid(s), preferably a sequence which contains secretory or leader sequences, pro-sequences, sequences which aid in purification, such as multiple histidine residues, or an additional sequence for stability during recombinant production.

The polypeptides may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one which is fused with another compound, such as polyethylene glycol, or (iv) one which is fused to a heterologous sequence such as additional amino acids which aid in purification or which enhance processivity. Such polypeptides are deemed to be within the scope of those skilled in the art from the teachings herein.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

Preferably, the polypeptides of the invention, including mutants, fragments and variants, demonstrate a functional activity such as an enzymatic activity described above or antigenicity.

The functional activity of polypeptides of the invention can be assayed by various methods. For example, in one embodiment where one is assaying for antigenicity, various immunoassays known in the art can be used, including but not limited to, competitive and non competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In addition, assays described herein and otherwise known in the art may routinely be applied to measure the ability of polypeptides of the invention to elicit an enzymatic activity.

Antibodies and Antibody Fusions

The present invention also includes antibodies that are capable of “specifically binding” to hLy6-BIG, and productions and uses thereof. The known capacity of an antibody to bind to an antigen is an example of “specific binding.” Such interactions are in contrast to non-specific binding between classes of compounds, irrespective of their chemical structure (such as the binding of proteins to nitrocellulose, etc.). Most preferably, the antibodies of the present invention exhibit “highly specific binding,” such that they will be incapable or substantially incapable of binding to closely related polypeptides (e.g., the proteins of FIGS. 2 and 12). Indeed, preferred antibodies of the present invention exhibit the capacity to bind to a polypeptide of any of Tables 1-19 (SEQ ID NOS: 2, 4, 6, 8, 10, 12, 14, 16, 18, 20, 22, 24, 26, 28, 30, 32, 34, 36, or 38) or a polypeptide encoded by a deposited clone, but are substantially incapable of binding the non-Ly6-BIG proteins of FIGS. 2 and 12; such antibodies are capable of highly specific binding to a polypeptide of any of Tables 1-19 (SEQ ID NOS: 2, 4, 6, 8, 10, 12, 14, 16, 18, 20, 22, 24, 26, 28, 30, 32, 34, 36, or 38) or a polypeptide encoded by a deposited clone, as that phrase is used herein. In preferred embodiments, antibodies of the invention do not include antibodies that bind to the non-Ly6-BIG proteins of FIGS. 2 and 12.

However, it is immediately apparent to one of ordinary skill that even antibodies that bind to other proteins, i.e., which are cross-reactive because they recognize an epitope (antigenic region) shared between a polypeptide of the invention and another polypeptide, are still useful for methods of the invention.

Antibodies of the invention include, but are not limited to, polyclonal, monoclonal, multispecific, human, humanized or chimeric antibodies, single chain antibodies, Fab fragments, F(ab′)2 fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In one embodiment, the present invention provides hybridoma cell lines expressing an antibody of the invention, the antibodies produced by these cells lines, and the polynucleotides encoding the antibodies. Such hybridomas are listed below and on page 7. These hybridomas were deposited with the American Type Culture Collection (“ATCC”) on the date listed on page 7, and given ATCC Deposit Numbers listed below and on page 7. The ATCC is located at 10801 University Boulevard, Manassas, Va. 20110-2209, USA. The ATCC deposits were made pursuant to the terms of the Budapest Treaty on the international recognition of the deposit of microorganisms for purposes of patent procedure.

| Hybridoma | Date of Deposit | Deposit Number |
|---|---|---|
| Hybridoma 7F6-E12-A5 | Feb. 25, 2005 | PTA-6611 |
| Hybridoma 31A3-D4-H4 | Feb. 25, 2005 | PTA-6612 |
| Hybridoma 26G6-C1-F1 | Oct. 21, 2005 | |
| Hybridoma 29F6-E7-G4 | Oct. 21, 2005 | |
| Hybridoma 5B8-1A9-E10 | Oct. 21, 2005 | |

Hybridoma 7F6-E12-A5 may be referred to herein as “7F6”, Hybridoma 31A3-D4-H4 may be referred to herein as “31A3”, Hybridoma 26G6-C1-F1 may be referred to herein as “26G6”, Hybridoma 29F6-E7-G4 may be referred to herein as “29F6”, and Hybridoma 5B8-1A9-E10 may be referred to herein as “5B8”.

The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

Most preferably for use in humans, the antibodies are human or humanized antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH region. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains.

Preferred antibodies in the therapeutic methods of the invention are those containing a deletion of the CH2 domain.

As used herein, the term “humanized” immunoglobulin or “humanized” antibody refers to an immunoglobulin comprising a human framework, at least one CDR from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. For example, a humanized immunoglobulin would not encompass a chimeric mouse variable region/human constant region antibody.

As used herein, the term “chimeric” antibody refers to an antibody whose heavy and light chains have been constructed, typically by genetic engineering, from immunoglobulin gene segments belonging to different species. For example, the variable (V) segments of the genes from a mouse monoclonal antibody may be joined to human constant (C) segments, such as gamma1 and/or gamma4. A typical therapeutic or diagnostic chimeric antibody is thus a hybrid protein comprising at least one V region (e.g., VH or VL) or the entire antigen-binding domain (i.e., VH and VL) from a mouse antibody and at least one C (effector) region (e.g., CH(CH1, CH2, CH3, or CH4) or CL (CL1, CL2, CL3, or CIA)) or the entire C domain (i.e., CH and CL) from a human antibody, although other mammalian species may be used. In some embodiments, especially for use in the therapeutic methods of the invention, chimeric antibodies contain no CH2 domain.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, or by size in contiguous amino acid residues. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their binding affinity to a polypeptide of the invention. Preferred binding affinities include those with a dissociation constant or Kd less than 5×10(−7) M, 10(−7) M, 5×10(−8) M, 10(−8) M, 5×10(−9) M, 10(−9) M, 5×10(−10) M, 10(−10) M, 5×10(−11) M, 10(−11) M, 5×10(−12) M, 10(−12) M, 5×10(−13) M, 10(−13) M, 5×10(−14) M, 10(−14) M, 5×10(−15) M, or 10(−15) M.

Antibodies of the present invention may also be described or specified in terms of their cross-reactivity. Antibodies that do not bind any other analog, ortholog, or homolog of a polypeptide of the present invention are included. Antibodies that bind polypeptides with at least a certain % identity (as described herein) to a polypeptide of the present invention are also included in the present invention, but such antibodies may also be excluded.

Antibodies of the present invention may also be described or specified in terms of their cross-reactivity, or lack thereof. Antibodies that do not bind any other analog, ortholog, or homolog of a polypeptide of the present invention are included. In some embodiments, antibodies of the present invention cross-react with homologs (e.g., murine, rat and/or rabbit homologs) of the polypeptides of the invention and the corresponding epitopes thereof, and in other embodiments, such antibodies are excluded (i.e., antibodies of the invention do not cross-react with any homologs).

In some embodiments, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein).

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

Antibody fusions of the invention also include fusions of the full length LY6-BIG proteins of the invention, or portions thereof, to the antibodies and antibody fragments described above. Antibody fusions of the invention are exemplified in Example 8 and Tables 20-21. The invention also includes polynucleotides encoding such antibody fusions.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

### Polynucleotides

Polynucleotides of the invention include, but are not limited to, polynucleotides described above and below. For example, polynucleotides of the invention include polynucleotides comprising, or alternatively consisting of, a nucleic acid encoding a polypeptide of any of Tables 1-19 (SEQ ID NOS: 2, 4, 6, 8, 10, 12, 14, 16, 18, 20, 22, 24, 26, 28, 30, 32, 34, 36, or 38), polynucleotides comprising, or alternatively consisting of, a nucleotide sequence of Table 1, 3, 5, 7, 9, 11, 13, 15, 17, 19, 21, 23, or 25 (SEQ ID NOS:1, 3, 5, 7, 9, 11, 13, 15, 17, 19, 21, 23, 25, 27, 29, 31, 33, 35, or 37), polynucleotides comprising, or alternatively consisting of, a nucleic acid encoding a polypeptide encoded by a nucleotide sequence of one of the deposited clones, polynucleotides comprising, or alternatively consisting of, a nucleotide sequence of one of the deposited clones, and/or mutants, fragments (e.g., portions), and variants thereof.

As described above, and further described below, polynucleotides of the invention also include, but are not limited to, polynucleotides comprising, or alternatively consisting of, nucleic acids encoding a mutant hLy6-BIG protein which comprise one or more substitutions corresponding to an amino acid residue(s) of an amino acid sequence of any of Tables 1-19 (SEQ ID NOS: 2, 4, 6, 8, 10, 12, 14, 16, 18, 20, 22, 24, 26, 28, 30, 32, 34, 36, or 38), polynucleotides comprising, or alternatively consisting of, nucleic acids which comprise one or more substitutions corresponding to a nucleotide sequence of Tables 1-19 (SEQ ID NOS:1, 3, 5, 7, 9, 11, 13, 15, 17, 19, 21, 23, 25, 27, 29, 31, 33, 35, or 37), polynucleotides comprising, or alternatively consisting of, nucleic acids encoding mutant hLy6-BIG proteins which comprise one or more substitutions corresponding to an amino acid residue(s) of a polypeptide encoded by a nucleotide sequence of one of the deposited clones, polynucleotides comprising, or alternatively consisting of, nucleic acids which comprise one or more substitutions corresponding to a nucleotide sequence of one of the deposited clones and/or mutants, fragments (e.g., portions), and variants thereof.

SEQ ID NOS:1, 3, 5, 7, 9, 11, 13, 15, 17, 19, 21, 23, 25, 27, 29, 31, 33, 35, or 37 and the translated SEQ ID NOS: 2, 4, 6, 8, 10, 12, 14, 16, 18, 20, 22, 24, 26, 28, 30, 32, 34, 36, or 38, are sufficiently accurate and otherwise suitable for a variety of uses well known in the art and described further below. For instance, SEQ ID NOS:1, 3, 5, 7, 9, 11, 13, 15, 17, 19, 21, 23, 25, 27, 29, 31, 33, 35, or 37 are useful for designing nucleic acid hybridization probes/primers that will detect and/or amplify nucleic acid sequences contained in SEQ ID NOS:1, 3, 5, 7, 9, 11, 13, 15, 17, 19, 21, 23, 25, 27, 29, 31, 33, 35, or 37, respectively, or the DNAs contained in the respective deposited clone. These probes/primers will also hybridize to/amplify nucleic acid molecules in tissue and cell samples, thereby enabling detection of the tissues and cells types expressing SEQ ID NOS:1, 3, 5, 7, 9, 11, 13, 15, 17, 19, 21, 23, 25, 27, 29, 31, 33, 35, or 37. Similarly, polypeptides identified from SEQ ID NOS: 2, 4, 6, 8, 10, 12, 14, 16, 18, 20, 22, 24, 26, 28, 30, 32, 34, 36, or 38, may be used, for example, to generate antibodies which bind specifically to the polypeptides of the invention.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NOS:1, 3, 5, 7, 9, 11, 13, 15, 17, 19, 21, 23, 25, 27, 29, 31, 33, 35, or 37 and the predicted translated amino acid sequence identified as SEQ ID NOS: 2, 4, 6, 8, 10, 12, 14, 16, 18, 20, 22, 24, 26, 28, 30, 32, 34, 36, or 38, but also a sample of plasmid DNA containing a DNA clone encoding the hLy6-BIG proteins of the invention deposited with the ATCC. The nucleotide sequence of the deposited clones can readily be determined by sequencing the deposited clones in accordance with known methods. The predicted amino acid sequences can then be verified from such deposits. Moreover, the amino acid sequence of the protein encoded by the deposited clone can also be directly determined by peptide sequencing or by expressing the protein in a suitable host cell containing the deposited DNA, collecting the protein, and determining its sequence.

The polynucleotides of the present invention may be in the form of RNA or in the form of DNA, which DNA includes cDNA, genomic DNA, and synthetic DNA. The DNA may be double stranded or single-stranded, and if single stranded may be the coding strand or non-coding (anti-sense) strand.

Nucleic acids encoding a polypeptide of any of Tables 1-19 (SEQ ID NOS: 2, 4, 6, 8, 10, 12, 14, 16, 18, 20, 22, 24, 26, 28, 30, 32, 34, 36, or 38) may substantially differ from the nucleotide sequences in Tables 1-19 (SEQ ID NOS:1, 3, 5, 7, 9, 11, 13, 15, 17, 19, 21, 23, 25, 27, 29, 31, 33, 35, or 37) or in the deposited clones due to the degeneracy of the genetic code. Of course, the genetic code is well known in the art. Thus, it would be routine for one skilled in the art to generate the degenerate polynucleotides described above.

The present invention particularly relates to polynucleotides which hybridize under stringent conditions to the hereinabove-described polynucleotides. The polynucleotides which hybridize to the hereinabove described polynucleotides in a preferred embodiment encode polypeptides which retain substantially the same functional activity as the polypeptide encoded by the nucleotide sequence of Tables 1-19 (SEQ ID NOS:1, 3, 5, 7, 9, 11, 13, 15, 17, 19, 21, 23, 25, 27, 29, 31, 33, 35, or 37) or the hLy6-BIG proteins encoded by the deposited clones.

In another aspect, the invention provides an isolated nucleic acid molecule comprising, or alternatively consisting of, a polynucleotide which hybridizes under stringent hybridization conditions to a portion of the polynucleotide in a nucleic acid molecule of the invention described above. Such hybridizing polynucleotides may not encode a polypeptide, and are still useful, for example, as probes or primers.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30 70 nt of the reference polynucleotide. Also intended is a polynucleotide hybridizing to at least about 15 nucleotides (nt), and more preferably at least about 20 nt, more preferably at least about 25 nt, still more preferably at least about 30 nt, and even more preferably about 30 70 (e.g., 30, 35, 40, 45, 50, 55, 60, 65, and/or 70 (of course, fragment lengths in addition to those recited herein are also useful)) nt of the reference polynucleotide. Alternatively, the polynucleotide may have at least 20 bases, preferably 30 bases, and more preferably at least 50 bases which hybridize to a polynucleotide of the present invention, as hereinabove described, and which may or may not encode a polypeptide. Of course, larger fragments 50 500 nt, 500-1000 nt, 1000-1500 nt, 1500-2000 nt, 2000-2500 nt, 2500-3000 nt, 3000-3500 nt in length are also useful in the present invention (see below). For example, such polynucleotides may be employed as probes for the full length polynucleotides, for example, for recovery or detection of the polynucleotide or as a PCR primer.

Of course, polynucleotides hybridizing to a larger portion of the reference polynucleotide (e.g. the deposited cDNA clone) or even to the entire length of the reference polynucleotide, are also useful as probes according to the present invention, as are polynucleotides corresponding to most, if not all, of the nucleotide sequence of the deposited clone or the nucleotide sequence as shown in Tables 1-19. By a portion of a polynucleotide of “at least 20 nt in length,” for example, is intended 20 or more contiguous nucleotides from the nucleotide sequence of the reference polynucleotide. As indicated, such portions are useful as a probe according to conventional DNA hybridization techniques or as primers for amplification of a target sequence by the hLy6-BIG protein chain reaction (PCR), as described herein.

Generating polynucleotides which hybridize to a portion of the nucleic acid molecules would be routine to the skilled artisan. For example, restriction endonuclease cleavage or shearing by sonication of a deposited clone could easily be used to generate DNA portions of various sizes which are polynucleotides that hybridize to a portion of the full length nucleic acid molecule. Alternatively, the hybridizing polynucleotides of the present invention could be generated synthetically according to known techniques.

The present invention is further directed to fragments of the isolated nucleic acid molecules described herein. By a fragment of an isolated nucleic acid molecule having the nucleotide sequence of a deposited cone, or a nucleotide sequence shown in Tables 1-19 is intended fragments at least about 15 nucleotides (nt), and more preferably at least about 20 nt, still more preferably at least about 30 nt, and even more preferably, at least about 40 nt in length which are useful as probes and primers as discussed herein. Of course, larger fragments 50 100 nt, 100-200 nt, 200-300 nt, 300-400 nt, 400-500 nt, 500-600 nt, 600-700 nt, 700-800 nt, 800-1000 nt, 1000-2000 nt, 2000-3000 nt, 3000-4000 nt in length are also useful according to the present invention as are fragments corresponding to most, if not all, of a nucleotide sequence of a deposited clone, or as shown in Tables 1-19. By a fragment at least 20 nt in length, for example, is intended fragments which include 20 or more contiguous bases from the nucleotide sequence of a deposited clone or the nucleotide sequence as shown in Tables 1-19.

Polynucleotide fragments and hybridizing polynucleotides may be from 15 to 4000 nucleotides in length such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1001, 1002, 1003, 1004, 1005, 1006, 1007, 1008, 1109, 1010, 1011, 1012, 1013, 1014, 1015, 1016, 1017, 1018, 1019, 1020, 1121, 1022, 1023, 1024, 1025, 1026, 1027, 1028, 1029, 1030, 1031, 1032, 1133, 1034, 1035, 1036, 1037, 1038, 1039, 1040, 1041, 1042, 1043, 1044, 1145, 1046, 1047, 1048, 1049, 1050, 1051, 1052, 1053, 1054, 1055, 1056, 1157, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 390, 4000, or more nucleotides in length.

Polynucleotides of the invention include variants which are at least 80%, 81%, 82%, 83%, 84%, 85%, 86%, 87%, 88%, 89%, 90%, 91%, 92%, 93%, 94%, 95% 96%, 97%, 98%, or 99% identical to the polypeptide-encoding or hLy6-BIG protein-encoding nucleotide sequences of Tables 1-19 (SEQ ID NOS:1, 3, 5, 7, 9, 11, 13, 15, 17, 19, 21, 23, 25, 27, 29, 31, 33, 35, or 37), or to the hLy6-BIG protein nucleic acids of the deposited clones, or to the polynucleotide fragments described above.

Thus, the invention includes, in part, polynucleotides which are at least 80%, 81%, 82%, 83%, 84%, 85%, 86%, 87%, 88%, 89%, 90%, 91%, 92%, 93%, 94%, 95% 96%, 97%, 98%, or 99% identical to (1) nucleic acid contained in a deposited clone described herein, (2) to a polynucleotide having a nucleotide sequence set out in Tables 1, 3, 5, 7, 9, 11, 13, 15, 17, 19, 21, 23, or 25 (SEQ ID NOS:1, 3, 5, 7, 9, 11, 13, 15, 17, 19, 21, 23, 25, 27, 29, 31, 33, 35, or 37), or (3) to a subportion of one of these polynucleotides. The invention further includes host cells which contain such nucleic acid molecules. The invention also includes compositions and mixtures (e.g., reaction mixtures) which contain one or more of these polynucleotides, as well as methods for producing polypeptides using these polynucleotides.

In many instances, the above described polynucleotides will encode polypeptides which have one or more activity associated with a polypeptide encoded by a deposited clone described herein or a polypeptide having an amino acid sequence set out in any of Tables 1-19 (SEQ ID NOS: 2, 4, 6, 8, 10, 12, 14, 16, 18, 20, 22, 24, 26, 28, 30, 32, 34, 36, or 38).

The variants may contain alterations in the coding regions, non coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5 10, 1 5, or 1 2 amino acids are substituted, deleted, or added in any combination are also preferred. Polynucleotide variants can be produced for a variety of reasons, e.g., to optimize codon expression for a particular host (change codons to those preferred by a particular bacterial host such as E. coli). Most highly preferred are nucleic acid molecules encoding an amino acid sequence encoded by a deposited clone, as described herein. Isolated nucleic acid molecules, particularly DNA molecules, are useful as probes and primers for producing the polypeptides of the invention, for example, by PCR or DNA shuffling.

Polynucleotides of the invention include polynucleotides comprising or consisting of nucleic acids encoding fragments of the polypeptides of Tables 1-19 (SEQ ID NOS: 2, 4, 6, 8, 10, 12, 14, 16, 18, 20, 22, 24, 26, 28, 30, 32, 34, 36, or 38) or the hLy6-BIG proteins encoded by the deposited clones.

Nucleic acids may encode fragments which are from 6 to 342 amino acids in length. Thus, nucleic acids may encode fragments which are 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, or 342 amino acids in length.

Nucleic acids may encode fragments which are 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, or 342 amino acids of the full length hLy6-BIG protein of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, or of the full length hLy6-BIG proteins encoded by the deposited clones, as described above.

Nucleic acids of the invention may encode fragments which contain a continuous series of deleted residues from the amino (N)- or the carboxyl (C)-terminus, or both, as described above.

Even if deletion of one or more amino acids from the N- and/or C-terminus of an encoded protein results in modification of loss of one or more biological functions of the encoded protein, other functional activities (e.g., enzymatic activities, antigenic activity, immunogenic activity) may still be retained. For example, the ability of shortened polypeptides to induce and/or bind to antibodies which recognize the complete forms of the polypeptides generally will be retained when less than the majority of the residues of the complete or mature polypeptide are removed from the N- and/or C-terminus. Whether a particular encoded polypeptide lacking N- and/or C-terminal residues of a complete polypeptide retains such immunologic activities can readily be determined by routine methods described herein and otherwise known in the art. It is not unlikely that an encoded fragment with a large number of deleted N- and/or C-terminal amino acid residues may retain some antigenic or immunogenic activities. In fact, peptides composed of as few as six amino acid residues may often evoke an immune response, as discussed below.

Nucleic acids may encode fragments which include unique regions, i.e., stretches of amino acids of the polypeptides or hLy6-BIG proteins of Tables 1-19 (SEQ ID NOS: 2, 4, 6, 8, 10, 12, 14, 16, 18, 20, 22, 24, 26, 28, 30, 32, 34, 36, or 38) that are less than 100% identical to corresponding stretches of amino acids in other proteins such as the non-Ly6-BIG proteins of FIGS. 2 and 12 (SEQ ID NOS:______), as described above. Unique regions of each encoded polypeptide of the invention are shown in the alignment in FIGS. 2 and 12, which indicate the identical and non-identical amino acids of the hLy6-BIG proteins of Tables 1-19 (SEQ ID NOS: 2, 4, 6, 8, 10, 12, 14, 16, 18, 20, 22, 24, 26, 28, 30, 32, 34, 36, or 38) (or the hLy6-BIG proteins encoded by a deposited clone) as compared to related polypeptides. Nucleic acids encoding fragments which contain unique regions are useful for generating highly specific antibodies of the invention, for example by DNA vaccination or by vaccination or screening using recombinant polypeptide. Thus, nucleic acids encoding fragments which contain unique regions are preferred for producing recombinant antigenic fragments of the invention. Additionally, nucleic acids encoding fragments which contain unique regions are especially useful for producing fusion proteins such as proteins produced by DNA shuffling. Using DNA shuffling, nucleic acids encoding fusion proteins are constructed which encode polypeptides comprising fragments from one or more hLy6-BIG proteins and which preferably have an enzymatic activity of a polypeptide or hLy6-BIG protein of any of Tables 1-19 (SEQ ID NOS: 2, 4, 6, 8, 10, 12, 14, 16, 18, 20, 22, 24, 26, 28, 30, 32, 34, 36, or 38) or the hLy6-BIG proteins encoded by a deposited clone.

Other nucleic acids encode fragments characterized by structural or functional attributes of the polypeptides of the invention, as described above. Such nucleic acids encode fragments which comprise alpha helix and alpha helix forming regions (“alpha regions”), beta sheet and beta sheet forming regions (“beta regions”), turn and turn forming regions (“turn regions”), coil and coil forming regions (“coil regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson Wolf program) of full length polypeptides (e.g., the polypeptides of Tables 1-19 (SEQ ID NOS: 2, 4, 6, 8, 10, 12, 14, 16, 18, 20, 22, 24, 26, 28, 30, 32, 34, 36, or 38)). Nucleic acids encoding certain preferred regions include, but are not limited to, those encoding regions of the aforementioned types identified by analysis of the amino acid sequence depicted in Tables 1-19 (SEQ ID NOS: 2, 4, 6, 8, 10, 12, 14, 16, 18, 20, 22, 24, 26, 28, 30, 32, 34, 36, or 38), such preferred regions include; Garnier Robson predicted alpha regions, beta regions, turn regions, and coil regions; Chou Fasman predicted alpha regions, beta regions, turn regions, and coil regions; Kyte Doolittle predicted hydrophilic and hydrophobic regions; Eisenberg alpha and beta amphipathic regions; Emini surface forming regions; and Jameson Wolf high antigenic index regions, as predicted using the default parameters of these computer programs. These structural or functional attributes can be generated using the various modules and algorithms of the DNA*STAR program set on default parameters.

Additional regions encoded by the polynucleotides of the invention are defined in FIGS. 9A-9S and 10A-10G and in Table 22.

Among preferred nucleic acids encoding fragments in this regard are those that encode fragments which comprise regions of the polypeptides that combine several structural features, such as several of the features set out above or below.

In another embodiment, nucleic acids may encode polypeptides which comprise or consist of one or more fragments (e.g., regions). For a nucleic acids encoding a polypeptide comprising or consisting of the amino acid sequence of two or more fragments (e.g., regions), the encoded fragments (e.g., regions) may be contiguous with one another. In one embodiment, the encoded fragments (e.g., regions) are not contiguous with one another, i.e., they are separated by one or more amino acid residues.

Preferably, the nucleic acids encode fragments (e.g., regions) which align with the corresponding regions of the full length polypeptide such that they are separated by the same number of amino acid residues as separate them in the full length polypeptide or the full length hLy6-BIG protein (e.g., the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, (or the hLy6-BIG proteins encoded by the deposited clones).

Nucleic acids may encode fragments containing antigenic regions (i.e., regions to which an antibody will bind; epitopes) of the polypeptides of the invention. Nucleic acids may encode antigenic regions as small as 6 amino acids.

The selection of nucleic acids encoding fragments bearing an antigenic region is described above. See, e.g., Sutcliffe, J. G., Shinnick, T. M., Green, N. and Learner, R. A., Science 219:660 666 (1983).

Nucleic acids encoding antigenic fragments preferably encode a sequence of at least seven, more preferably at least nine and most preferably between about 15 to about 30 amino acids. However, nucleic acids may encode a larger portion such as about 30 to about 50 amino acids, or any length up to and including the entire amino acid sequence of a polypeptide of the invention.

In the present invention, nucleic acids may encode antigenic fragments which preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred nucleic acids encoding polypeptides comprising antigenic fragments are at least 10, 15, 20, 25, 30, 35, 40, 45, 50, 55, 60, 65, 70, 75, 80, 85, 90, 95, or 100 amino acid residues in length. Additional non exclusive preferred nucleic acids which encode antigenic fragments include nucleic acids encoding the fragments disclosed herein, as well as portions thereof. Preferred antigenic fragments include the fragments disclosed herein, as well as any combination of two, three, four, five or more of these fragments.

Polynucleotides comprising nucleic acids encoding one or more antigenic fragments may encode a carrier protein, such as an albumin, either separately or fused in frame the antigenic fragment.

Polynucleotides of the invention may comprise or consist of nucleic acids encoding variants of the full length hLy6-BIG protein (the polypeptides of Tables 1-19 (SEQ ID NOS: 2, 4, 6, 8, 10, 12, 14, 16, 18, 20, 22, 24, 26, 28, 30, 32, 34, 36, or 38) with or without the N-terminal Met residue and with or without the signal sequence, variants of the polypeptides encoded by the deposited clones, and variants of the fragments described above. Encoded variants include polypeptides which are at least 80%, 81%, 82%, 83%, 84%, 85%, 86%, 87%, 88%, 89%, 90%, 91%, 92%, 93%, 94%, 95% 96%, 97%, 98%, or 99% identical to a polypeptide encoded by a deposited clone, to a polypeptide of any of Tables 1-19 (SEQ ID NOS: 2, 4, 6, 8, 10, 12, 14, 16, 18, 20, 22, 24, 26, 28, 30, 32, 34, 36, or 38), or to a fragment described above.

The invention includes nucleic acids encoding variants which may show a functional activity. Preferably, nucleic acids encode variants which demonstrate a functional activity such as antigenicity or an activity described above.

Polynucleotide variants include nucleotide deletions, insertions, inversions, repeats, and substitutions. Polynucleotide variants also include nucleic acids encoding polypeptide deletions, insertions, inversions, repeats, and substitutions (e.g., conservative substitutions, non-conservative substitutions, type substitutions (for example, substituting one hydrophilic residue for another hydrophilic residue, but not a strongly hydrophilic for a strongly hydrophobic, as a rule), primary shifts, primary transpositions, secondary transpositions, and coordinated replacements).

Nucleic acids may encode polypeptide variants in which more than one amino acid (e.g., 2, 3, 4, 5, 6, 7, 8, 9 and 10) is substituted with another amino acid as described above (either conservative or nonconservative). The substituted amino acids can occur in the full length form of the polypeptide, as well as in the fragments described above.

Nucleic acids may encode variants which contain at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of increasing preference, it is preferable for a nucleic acid to encode a variant containing at least one, but not more than 10, 9, 8, 7, 6, 5, 4, 3, 2 or 1 amino acid substitutions. In specific embodiments, the number of additions, substitutions, and/or deletions in the encoded polypeptide (e.g., the full length form and/or fragments described herein), is 1 5, 5 10, 5 25, 5 50, 10 50 or 50 150. Encoded variants may preferably contain conservative amino acid substitutions.

Nucleic acids preferably encode variants containing the amino acid substitutions described herein (see above).

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Of additional special interest are also substitutions of charged amino acids with another charged amino acid or with neutral amino acids. This may result in proteins with improved characteristics such as less aggregation. Prevention of aggregation is highly desirable. Aggregation of proteins can result in a reduced activity.

Polynucleotides of the invention may be altered by being subjected to random mutagenesis by error-prone PCR, random nucleotide insertion or other methods prior to recombination. Polynucleotides of the invention may be produced by DNA shuffling, gene-shuffling, motif-shuffling, exon-shuffling, and/or codon-shuffling (collectively referred to as “DNA shuffling”). DNA shuffling involves the assembly of two or more DNA segments by homologous or site-specific recombination to generate variation in the polynucleotide sequence. DNA shuffling may be employed to modulate the activities of polypeptides of the invention, such methods can be used to generate polypeptides with altered activity. See, generally, U.S. Pat. Nos. 5,605,793; 5,811,238; 5,830,721; 5,834,252; 5,837,458; and 6,444,468; and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). Polynucleotides of the invention encode contain one or more components, motifs, sections, parts, domains, fragments, etc., of a polypeptide of the invention joined to one or more components, motifs, sections, parts, domains, fragments, etc. of one or more heterologous molecules, preferably the non-Ly6-BIG proteins in FIGS. 2 and 12 and/or the Ly6-BIG proteins of Tables 1-19 (SEQ ID NOS: 2, 4, 6, 8, 10, 12, 14, 16, 18, 20, 22, 24, 26, 28, 30, 32, 34, 36, or 38).

Nucleic acids encoding fragments, mutants, variants, or full length polypeptides of the invention may be “free standing,” or comprised within a larger polynucleotide of which the nucleic acid encoding the fragment, mutant, variant, or full length polypeptide forms a part or region.

Thus, polynucleotides may encode one or more additional amino acids and/or one or more heterologous sequences such as those described herein. For instance, polynucleotides may comprise a codon for methionine added to the 5′ end of the nucleic acid encoding the polypeptide, such that the encoded polypeptide comprises a Met residue at the N-terminus, thus allowing for recombinant expression. Also, the polynucleotide may comprise a nucleic acid encoding additional a sequence of amino acids, particularly charged amino acids, which may fused to the N terminus of the encoded polypeptide to improve stability and persistence, in the host cell, during purification, or during subsequent handling and storage. A preferred polynucleotide encodes a fusion protein comprising a heterologous region from immunoglobulin that is useful to solubilize proteins.

Thus, polynucleotides may comprise the nucleic acids above and may also encode one or more additional amino acids and/or one or more heterologous polypeptides. Heterologous polypeptides include secretory or leader sequences, pro-sequences, tags or other sequences which aid in purification, such as multiple histidine residues, or an additional sequence for stability during recombinant production.

Preferably, polynucleotides encode polypeptides which demonstrate a functional activity such as an enzymatic activity described above or antigenicity.

As indicated, nucleic acid molecules of the present invention which encode a polypeptide of the invention may include, but are not limited to those encoding the amino acid sequence of the polypeptide (e.g., full length, fragment, mutant, or variant) by itself; the coding sequence for the polypeptide and additional sequences, such as those encoding the leader or secretory sequence, such as a pre, or pro or prepro protein sequence; the coding sequence of the polypeptide, with or without the aforementioned additional coding sequences, together with additional, non coding sequences, including for example, but not limited to introns and non coding 5′ and 3′ sequences, such as the transcribed, non translated sequences that play a role in transcription, mRNA processing, including splicing and polyadenylation signals for eucaryotic expression, for example ribosome binding and stability of mRNA; an additional coding sequence which codes for additional amino acids, such as heterologous sequences, for example those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa histidine peptide, such as the tag provided in a pQE vector (Qiagen, Inc.), among others, many of which are commercially available. As described in Gentz et al., Proc. Natl. Acad. Sci. USA 86:821 824 (1989), for instance, hexa histidine provides for convenient purification of the fusion protein. The “HA” tag is another peptide useful for purification which corresponds to an epitope derived from the influenza hemagglutinin protein, which has been described by Wilson et al., Cell 37: 767 (1984). Other such nucleic acids encoding fusion proteins include those encoding a polypeptide of the invention fused to Fc at the N- or C terminus.

Polynucleotides of the invention also include those encoding antibodies and antibody fusions of the invention.

### Binding Molecules

The invention includes “binding molecules,” which can be used in methods of treating autoimmune disorders, cancer, AIDS, and other disorders as described herein. A binding molecule comprises, consists essentially of, or consists of at least one binding domain which, either alone or in combination with one or more additional binding domains, specifically binds to a target gene product (such as a messenger RNA, a protein, an antigen or other binding partner), e.g., a polynucleotide encoding a Ly6-BIG polypeptide or a fragment thereof or the encoded Ly6-BIG polypeptide or fragment or variant thereof. For example, in various embodiments, a binding molecule comprises one or more antisense nucleic acids, one or more siRNA molecules, one or more ribozymes, one or more immunoglobulin antigen binding domains, one or more binding domains of a receptor molecule which, either alone or together, specifically bind a ligand, or one or more binding domains of a ligand molecule which, either alone or together, specifically bind a receptor. Nucleic acid binding molecules are described in more detail below. In certain embodiments, a binding molecule comprises, consists essentially of, or consists of at least two binding domains, for example, two, three, four, five, six, or more binding domains. Each binding domain may bind to a target molecule separately, or two or more binding domains may be required to bind to a given target, for example, a combination of an immunoglobulin heavy chain and an immunoglobulin light chain.

Binding molecules, e.g., binding polypeptides, e.g., Ly6-BIG-specific antibodies, used in the diagnostic and treatment methods disclosed herein may comprise, consist essentially of, or consist of two or more subunits thus forming multimers, e.g., dimers, trimers or tetramers. For example, certain binding molecules comprise a polypeptide dimer, typically, a heterodimer comprising two non-identical monomeric subunits. Other binding molecules comprise tetramers, which can include two pairs of homodimers, e.g., two identical monomeric subunits, e.g., an antibody molecule consisting of two identical heavy chains and two identical light chains.

Certain binding molecules, e.g., binding polypeptides, to be utilized in the diagnostic and treatment methods disclosed herein comprise at least one amino acid sequence derived from an immunoglobulin. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the starting sequence. Alternatively, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence, as described in more detail below.

Preferred binding polypeptides comprise, consist essentially of, or consist of an amino acid sequence derived from a human amino acid sequence. However, binding polypeptides may comprise one or more contiguous amino acids derived from another mammalian species. For example, a primate heavy chain portion, hinge portion, or binding site may be included in the subject binding polypeptides. Alternatively, one or more murine-derived amino acids may be present in a non-murine binding polypeptide, e.g., in an antigen binding site of a binding molecule. In therapeutic applications, preferred binding molecules to be used in the methods of the invention are not immunogenic in the animal to which the binding polypeptide is administered.

It will also be understood by one of ordinary skill in the art that the binding polypeptides for use in the diagnostic and treatment methods disclosed herein may be modified such that they vary in amino acid sequence from the naturally occurring binding polypeptide from which they were derived. For example, nucleotide or amino acid substitutions leading to conservative substitutions or changes at “non-essential” amino acid residues may be made.

In certain embodiments, a binding polypeptide for use in the methods of the invention comprises an amino acid sequence or one or more moieties not normally associated with that binding polypeptide. Exemplary modifications are described in more detail below. For example, a binding polypeptide of the invention may comprise a flexible linker sequence, or may be modified to add a functional moiety (e.g., PEG, a drug, a toxin, or a label).

A binding polypeptide for use in the methods of the invention may comprise, consist essentially of, or consist of a fusion protein. Fusion proteins are chimeric molecules which comprise a binding domain with at least one target binding site, and at least one heterologous portion.

A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a genotypically distinct entity from that of the rest of the entity to which it is being compared. For instance, a heterologous polynucleotide or antigen may be derived from a different species origin, different cell type, or the same type of cell of distinct individuals.

The term “ligand binding domain” or “ligand binding portion” as used herein refers to any native receptor (e.g., cell surface receptor) or any region or derivative thereof retaining at least a qualitative ligand binding ability, and preferably the biological activity of a corresponding native receptor.

The term “receptor binding domain” or “receptor binding portion” as used herein refers to any native ligand or any region or derivative thereof retaining at least a qualitative receptor binding ability, and preferably the biological activity of a corresponding native ligand.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### Uses

As mentioned previously, the polypeptides, polynucleotides and antibodies of the invention have therapeutic and diagnostic uses in cancer, autoimmunity, neurological disorders, bone disease and regenerative medicine.

Such uses include cancer therapy including the isolation/purification of hematopoietic stem cells for use in bone marrow and peripheral stem cell transplantation, genetic therapy, autoimmune diseases, treating other diseases of the immune system as described herein, and in AIDS and other infectious diseases, cancer.

### EXAMPLES

### Example 1

### Discovery of a Human Homolog of Mouse SCA-1 (Ly6A) (Genbank Accession NM_{—}010738)

The mouse genomic region corresponding to SCA-1 (chr15:76746602-76748941 on mouse genome build 32) was compared by TBLASTX (Altschul, Stephen F., et al., (1997), “Gapped BLAST and PSI-BLAST: a new generation of protein database search programs”, Nucleic Acids Res. 25:3389-3402) to the syntenic region of the human genome (chr8:144359932-144746492 on human genome build 33). The significant protein alignments were used as a starting point to designate exons. From the human genomic segment, the DNA was analyzed for splice sites by NetGene2 (S. M. Hebsgaard, et al., Nucleic Acids Research, 1996, Vol. 24, No. 17, 3439-3452. Brunak, S., et al., Journal of Molecular Biology, 1991, 220, 49-65.) Most likely splice sites flanking the proposed exons were used to define the exon boundaries. Comparison of mouse and human protein sequences was done using the BLAST program as described above. To further define the transcript, the genomic region after the stop codon was extended until it included the likely polyadenylation site as defined by the consensus sequence “AATAAA” (SEQ ID NO:______). See FIG. 2 for alignment of mouse and human sequences.

### Example 2

### Discovery of Novel Ly6 Family Members in Human Chromosome 8q24.3 Region

Human chromosome 8q24.3 (chr8:137804827-146274826 on human genome build 33) was taken and translated into all 6 reading frames of protein sequences and generated as a sequence database for PSI-BLAST comparison (see above reference). Known Ly6 protein sequences were added in to form the basis of the profile. Mouse SCA-1 protein sequence (Genbank accession NP_{—}034868) was taken as the query sequence to perform the profile searching. Four rounds of iterative searching were performed and the resulting sequences were analyzed as perspective novel Ly6 members. The new sequences were compared to the NCBI non-redundant database to determine if they were already known or potentially novel sequences. Those fragments that were determined to be novel (not matching to any known protein) were put together based on proximity and orientation on the genome and alignment to the profile and assembled into gene models. These models were then analyzed for the UPAR/Ly6 (Pfam accession PF00021; Pfam is a large collection of multiple sequence alignments and hidden Markov models covering many common protein families) motif by use of the HMMER package for hidden Markov model profile searching. (Profile hidden Markov models (profile HMMs) can be used to do sensitive database searching using statistical descriptions of a sequence family's consensus. HMMER is a freely distributable implementation of profile HMM software for protein sequence analysis.) Protein sequences that matched the Ly6 motif and had previously been shown to be novel are included herein. See, e.g., FIGS. 2 and 12)

### Example 3

### Protocols for PCR Amplification of cDNA

### PCR

1 ul 5 mM dNTP (Roche, supplied as 10 mM stock)
1 ul 5 uM forward primer
1 ul 5 uM reverse primer
5 ul 5×HS buffer
0.5 ul Taq polymerase (Promega)
x ul cDNA or water or GAPDH plasmid
q.s. to 25 ul with water
1×HS Buffer contains:
67 mM Tris-HCl, pH 8.8
4 mM MgC12
16 mM (NH4)₂SO4
33.2 ug/mL BSA
PCR Cycling parameters:

| | | | |
|---|---|---|---|
| Step 1 | 94° | 3 min | 1 cycle |
| Step 2 | 94° | 30 sec | 35 cycles |
| | 56° | 30 sec | |
| | 72° | 1 min | |
| Step 3 | 4° | hold | 1 cycle |

Primers:

GAPDH forward primer (MB2040)
(SEQ ID NO: _)
5′ ACCACAGTCCATGCCATCAC 3′
GAPDH reverse primer (MB2039)
(SEQ ID NO: _)
5′ TCCACCACCCTGTTGCTGTA 3′

Expected size of GAPDH product: 482 bp from cDNA, 586 bp from genomic DNA
PCf1, PCf2, PCf3, PCr1, and PCr2 are shown in FIG. 1.
   cDNA Synthesis

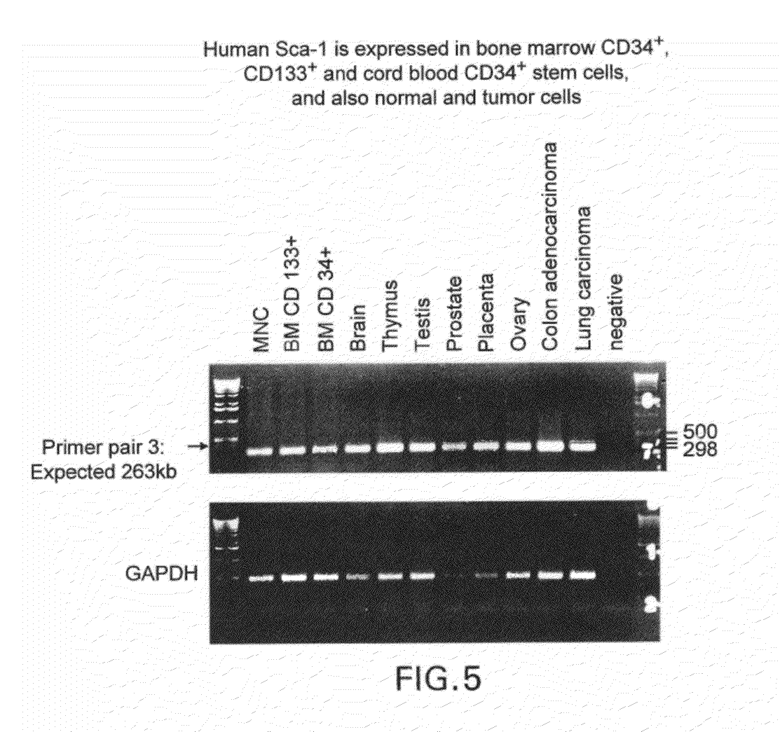

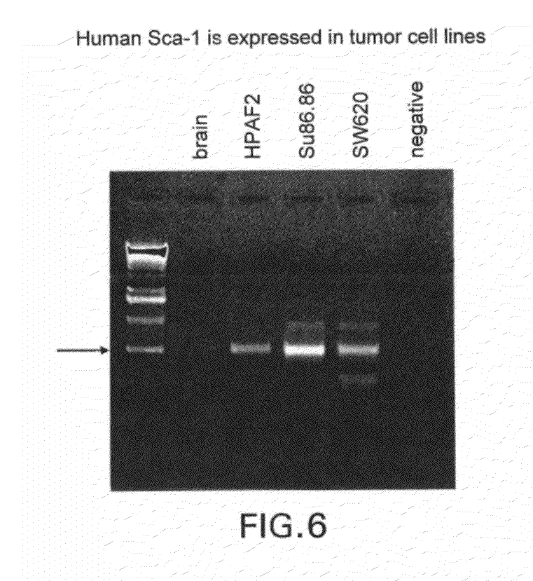

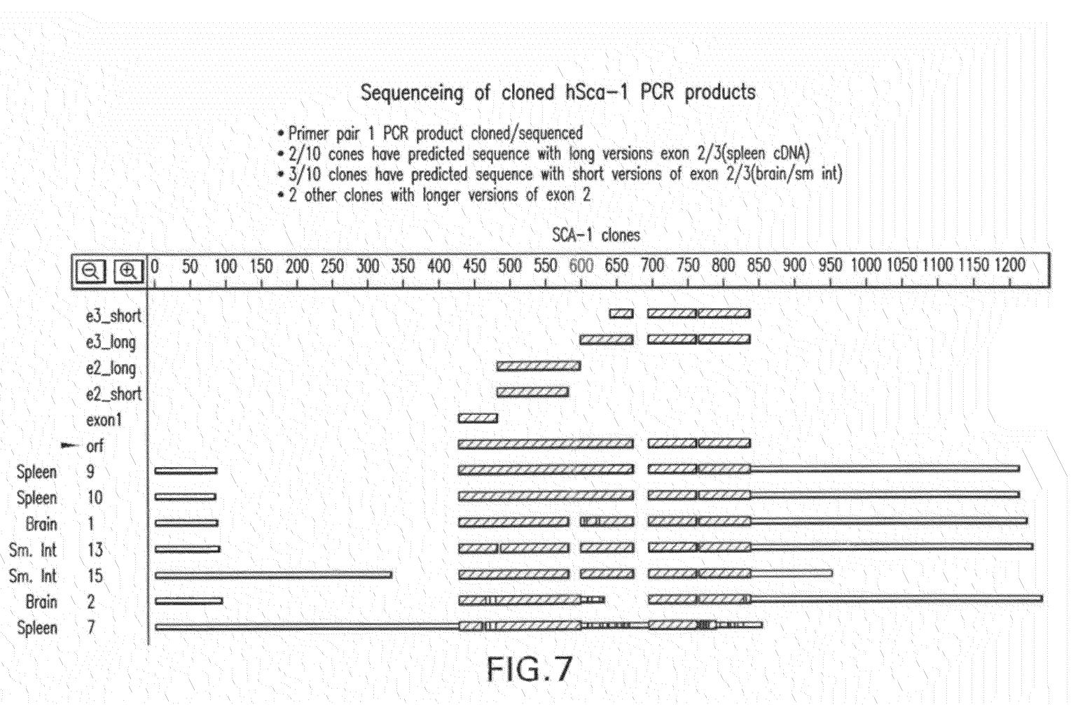

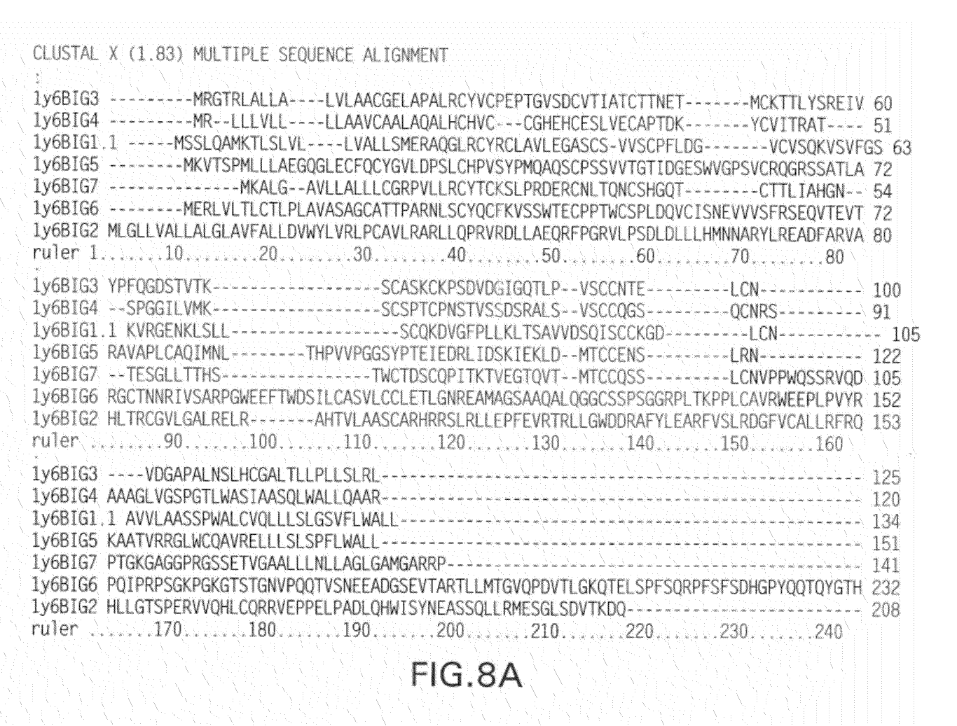

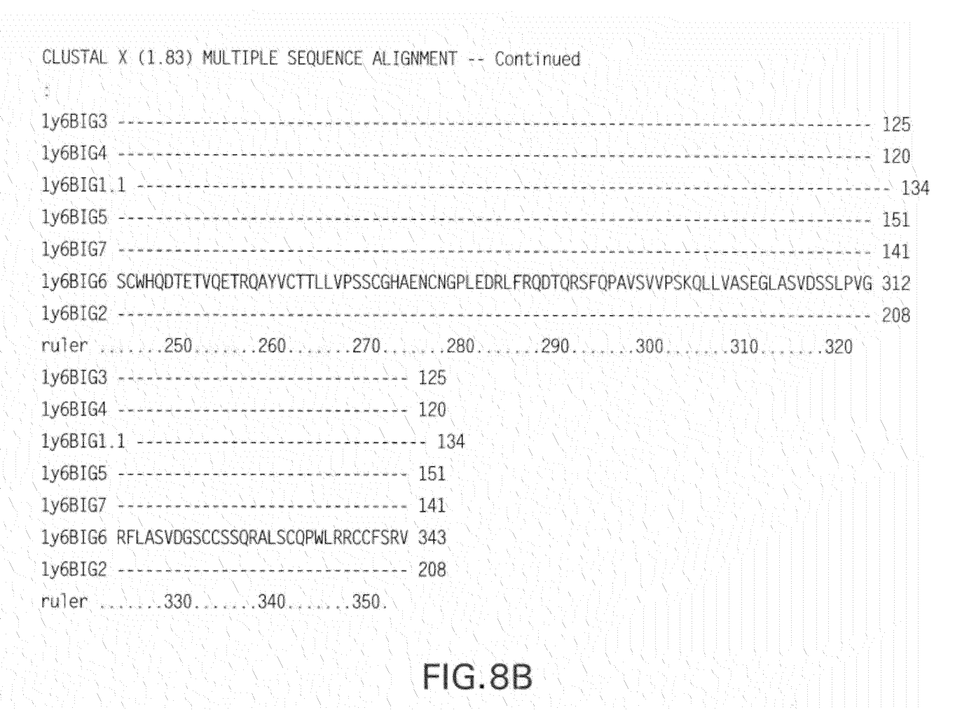

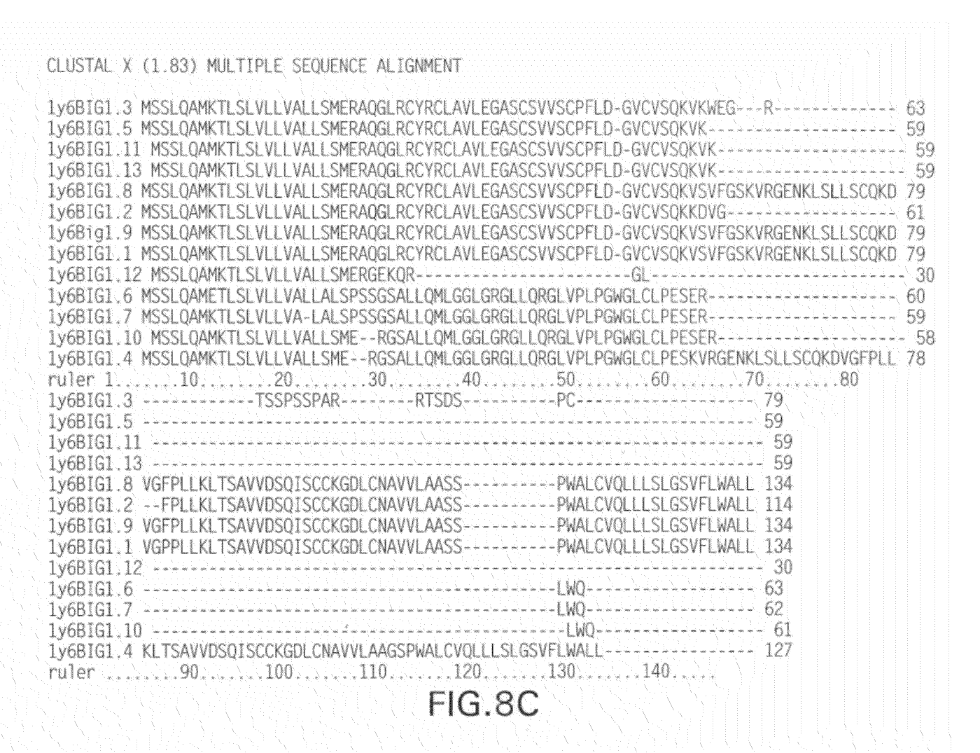

cDNA synthesis was performed using the SuperScript First-Strand Synthesis System for RT-PCR from Invitrogen to make the cDNA from bone marrow cells and mononuclear cells, data shown in FIG. 5. RNA for this synthesis was purchased from AllCells. This kit was also used to prepare the cDNAs from the tumor cell lines in FIG. 6, from RNA prepared at BiogenIdec using Qiagen RNEasy Maxi Kits.

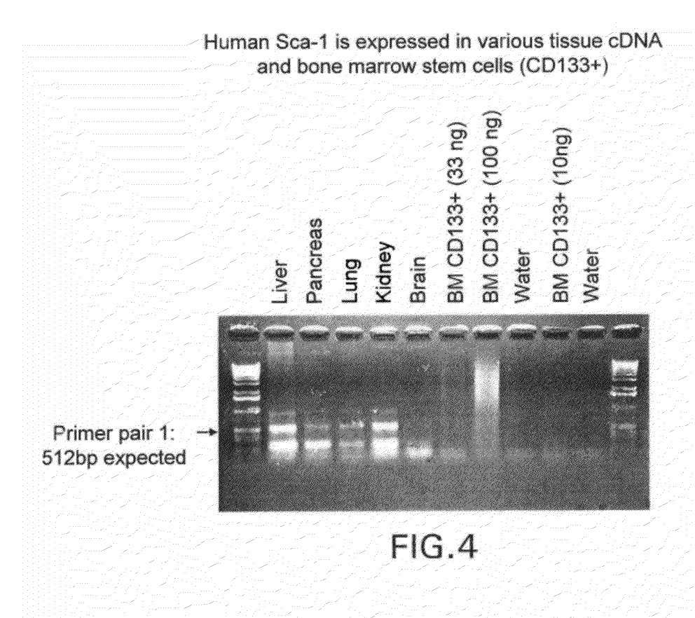

Other cDNAs used for PCR were purchased from Clontech (Multiple Tissue cDNA Panels I and II), except for the bone marrow cDNA in FIG. 4, which was obtained from AllCells, and the three brain sections: occipital cortex, temporal cortex, and cerebral cortex (results in FIG. 3) which were purchased from BioChain Institute, Inc.

### Example 4

### Purification of Hematopoietic Stem Cells

By using hLy6-BIG antibodies of the invention (e.g., hLy6-BIG1 antibodies), it is now possible to identify and purify hematopoietic stem cells (HSC) that are at least, or even more, primitive or totipotent as hematopoietic stem cells isolated using current protocols (i.e., CD34+ and/or CD133+). HSC are useful, e.g., for bone marrow transplantation, peripheral stem cell transplantation, and engraftment of patients.

Hematopoietic stem cells (HSCs) are clonogenic cells, which possess the properties of both self-renewal and multilineage potential giving rise to all types of mature blood cells. HSCs are the critical subset of cells in the hematopoietic system that undergo proliferation and differentiation to produce mature blood cells of various lineages while still maintaining their capacity for self-renewal. Hematopoiesis is a dynamic process with significant complexity in which the HSCs give rise to cells of both the myeloid and lymphoid lineages. In addition, HSCs have the ability to self-renew to produce more HSCs. This property allows HSCs to repopulate the bone marrow of lethally irradiated congenic hosts (a host that differs from another with respect to a small chromosomal segment). It is known that HSCs give rise to lymphoid and myeloid cells. Lymphoid cells will further differentiate into T, B, or NK cells. Myeloid cells will further differentiate into granulocyte, monocyte, mega-karyocyte, or erythrocyte cells. Recent reports indicate that murine HSCs also have the potential to trans-differentiate into multiple non-hematopoietic tissues. This suggests that HSCs have greater developmental potential than assumed previously. However, the underlying mechanisms of maintenance of multipotentiality in HSCs remain largely unknown. It is desired to have methods available for understanding such mechanisms.

Differentiation is the complex of changes involved in the progressive diversification of the structure and functioning of the cells of an organism. For a given line of cells, differentiation results in a continual restriction of the types of transcription that each cell can undertake.

Early hematopoiesis is a process of progressive restriction of developmental potential, accompanied with a hierarchical array of self-renewing and multipotent HSCs, non-self-renewing but multipotent progenitors (MPPs), and lineage restricted common lymphoid progenitors (CLPs) or common myeloid progenitors (CMPs). However, the mechanism behind this progressive restriction in developmental potential is not clear. As stated, the hematopoietic system includes HSC, MPP, CLP, and CMP populations. When grouped together, these four cell populations can be referred to as bone marrow stem cells, since all of these populations can be found in the bone marrow.

Early HSC development displays a hierarchical arrangement. The arrangement starts from long-term (LT-) HSCs, which have extensive self-renewal capability. Next is the expansion state, corresponding to short-term (ST-) HSCs (having limited self-renewal ability) and proliferative multipotent progenitor (MPP) (having multipotent potential but no self-renewal capability). MPP is also a stage of priming or preparation for differentiation. MPP differentiates and commits to either common lymphoid progenitor (CLP), which gives rise to all the lymphoid lineages, or common myeloid progenitor (CMP), which produces all the myeloid lineages. During this process, the more primitive population gives rise to a less primitive population of cells, which is unable to give rise to a more primitive population of cells. The intrinsic genetic programs that control these processes, including multipotential, self-renewal, and expansion (or transient amplification) of HSCs, and lineage commitment from MPP to CLP or CMP, remain largely unknown.

A number of review articles have been published addressing the phenotype of cells in hematopoietic lineages. Overall development of the hematolymphoid system is discussed in Orkin (1996) Curr. Opin. Genet. Dev. 6:597-602. The role of transcriptional factors in the regulation of hematopoietic differentiation is discussed in Georgopoulos et al. (1997) Annu. Rev. Immunol. 15:155-176; and Singh (1996) Curr. Opin. Immunol. 8:160-165.

The phenotype of hematopoietic stem cells is discussed in Morrison & Weissman (1994) Immunity 1, 661-673; Spangrude et al. (1988) Science 241, 58-62; Enver et al. (1998) Blood 92, 348-351; discussion 352; Uchida et al. (1994) Blood 83, 3758-3779, Morrison et al. The aging of hematopoietic stem cells. Nat Med 2, 1011-1016 (1996).

The phenotype of a common lymphoid progenitor cell is discussed by Kondo et al. (1997) Cell 91, 661-672. The role of Bc1-2 in lymphopoiesis is discussed in Akashi et al. (1997) Cell 89, 1033-1041. Lineage commitment and maturation is discussed by Metcalf (1998) Blood 92, 345-347; discussion 352. Mice defective in two apoptosis pathways in the myeloid lineage develop acute myeloblastic leukemia; Traver et al. (1998) Immunity 9, 47-57 (1998). Multipotent progenitors in acute myelogenous leukemia are described by Miyamoto, et al. (1996) Blood 87, 4789-4796.

Hematopoietic stem cells are characterized by both the presence of markers associated with specific epitopic sites identified by antibodies and the absence of certain markers. They may be further characterized by the level of a particular marker on the cell surface. It is not necessary that selection is achieved with a marker specific for hematopoietic stem cells. By using a combination of negative selection (removal of cells) and positive selection (isolation of cells), a substantially homogeneous hematopoietic stem cell composition can be achieved. See U.S. Pat. No. 5,087,570.

The isolation process will initially use a “relatively crude” separation to remove major cell families from the bone marrow or other hematopoietic cell source. For example, magnetic bead separations may be used initially to remove large numbers of cells, namely major cell populations of the hematopoietic system such as T-cells, various lineages, such as B-cells, both pre-B and B-cells, granulocytes, myelomonocytic cells, and platelets, or minor cell populations, such as megakaryocytes, mast cells, eosinophils and basophils. Generally, at least about 70%, usually 80% or more of the total hematopoietic cells will be removed. It is not essential to remove every dedicated cell class, particularly the minor population members, and the platelets and erythrocytes, at the initial stage. Since there will be positive selection at the end of the protocol, the dedicated cells will be left behind. However, it is preferable that there be positive selection for all of the cell lineages, so that in the final positive selection the number of dedicated cells present is minimized.

Murine hematopoietic stem cells may be characterized by having a hematopoietic stem cell antigen recognized by an antibody referred to as Sca-1, which monoclonal antibody is produced by the hybridoma E13 161-7 (Blood, 1986) 67:842), or 12-8, reported by Dr. Irving Bernstein, Fred Hutchinson Cancer Center, Seattle, Wash. In addition, the cells are found to lack antigenic markers for various mature hematopoietic lineages (Lin-), such as the surface markers associated with pre-B and B-cells, identified by the monoclonal antibody to the B220 antigen RA3-6B2, the marker associated with granulocytes identified by the RB6 8C5 anti-Gr-1 antibody, the marker associated with myelomonocytic cells identified by the Mac-1 antibody, and the CD4 and CD8 markers associated with T-cells. In addition, the cells contain significant but low levels of the cell surface differentiation antigen Thy-1. The human equivalents are used in the methods of the invention.

In order initially to obtain the subject hematopoietic stem cells, it is necessary to isolate the rare pluripotent hematopoietic stem cell from the other cells in bone marrow or other hematopoietic source. Initially, bone marrow cells may be obtained from a source of bone marrow, e.g. tibiae, femora, spine, fetal liver, and other bone cavities. Other sources of hematopoietic hematopoietic stem cells include fetal liver, fetal and adult spleen, yolk sac blood islands and the blood.

For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include HEPES, phosphate buffers, lactate buffers, etc.

Various techniques may be employed to separate the cells to initially remove cells of dedicated lineage. Monoclonal antibodies are particularly useful for identifying markers. The antibodies may be attached to a solid support to allow for separation. The separation techniques employed should maximize the retention of viability of the fraction to be collected. For “relatively crude” separations, that is, separations where up to 10%, usually not more than about 5%, preferably not more than about 1%, of the total cells present having the marker, may remain with the cell population to be retained, various techniques of differing efficacy may be employed. The particular technique employed will depend upon efficiency of separation, cytotoxicity of the methodology, ease and speed of performance, and necessity for sophisticated equipment and/or technical skill. Procedures for separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate. Techniques providing accurate separation include fluorescence activated cell sorters, which can have varying degrees of sophistication, e.g. a plurality of color channels, low angle and obtuse light scattering detecting channels, impedance channels, etc.

As exemplary of the subject method, in a first stage after incubating the cells from the bone marrow for a short period of time at reduced temperatures, generally −10.degree. to 10.degree. C., with saturating levels of antibodies specific for T-cell determinants, the cells are washed with a fetal calf serum (FCS) cushion. The washed cells are then suspended in a buffer medium as described above and separated by means of the antibodies for the T-cell determinants.

Conveniently, the antibodies may be conjugated with markers, such as magnetic beads, which allow for direct separation, biotin, which can be removed with avidin bound to a support, fluorescers, e.g. fluorescein, which can use a fluorescence activated cell sorter, or the like, to allow for ease of separation of the T-cells from the other cells. Any technique may be employed which is not detrimental to the viability of the remaining cells.

Once the cells bound to the antibodies are removed, they may then be discarded. The remaining cells may then be incubated for a sufficient time at reduced temperature with a saturating level of antibodies specific for one or a mixture of cell differentiation antigens. The same or different mechanism for selecting for these cells as was used for removing the T-cells may be employed, where in the subject step, it is intended to use the unbound cells in subsequent stages.

The cells selected for as having the cell differentiation antigen are then treated successively or in a single stage with antibodies specific for the B-cell lineage, myelomonocytic lineage, the granulocytic lineage, the megakaryocytic lineage, platelets, erythrocytes, etc., although minor lineages may be retained, to be removed later. The cells binding to these antibodies are removed as described above, with residual cells desirably collected in a medium comprising fetal calf serum.

The residual cells are then treated with labeled antibodies selective for hematopoietic stem cells, the antibodies hLy6-BIG (e.g., hLy6-BIG1 antibodies) and Thy-1^{lo}, where the labels desirably provide for fluorescence activated cell separation (FACS). Multi-color analysis may be employed at this stage or previously. The cells are separated on the basis of an intermediate level of staining for the cell differentiation antigen, a high level of staining for hLy6-BIG (e.g., hLy6-BIG1) and selected against dead cells and T-cells by providing for dyes associated with dead cells and T-cells as against hematopoietic stem cells. Desirably, the cells are collected in a medium comprising fetal calf serum or the equivalent. Other techniques for positive selection may be employed, which permit accurate separation, such as affinity columns, and the like. The method should permit the removal to a residual amount of less than about 1% of the non-stem cell populations.

The particular order of separation is not critical to this invention, but the order indicated is preferred. Preferably, cells will be initially separated by markers indicating unwanted cells, negative selection, followed by separations for markers or marker levels indicating the cells belong to the hematopoietic stem cell population, positive selection.

Compositions having greater than 90%, usually greater than about 95%, of hematopoietic stem cells may be achieved in this manner, where the desired hematopoietic stem cells are identified by having a low level of the Thy-1 cell differentiation antigen, being negative for the various lineage associated antigens and being positive for hLy6-BIG (e.g., hLy6-BIG1).

The hematopoietic stem cells appear as medium-size, lymphoid and round, intermediate in size between bone marrow lymphocytes and large myeloid cells. They are further distinguished by being late forming CFUs, which correlate with hematopoietic stem cells, whereby late forming is intended colonies of substantial size, at least about 2.+−.0.8 mm at day 12, while colonies at day 8, if any, are generally less than about 0.5.+−.0.2 mm.

A pluripotent hematopoietic stem cell may be defined as follows: (1) gives rise to progeny in all defined hematolymphoid lineages; and (2) limiting numbers of cells are capable of fully reconstituting a lethally irradiated host from which the cells are obtained. Fewer than 100 cells, usually fewer than 75 cells, more usually fewer than 50 cells, and as few as about 20 cells, or possibly as few as a single cell, are able to fulfill the conditions indicated above. Furthermore, the subject cells based on analysis of bone marrow cells appear to be in a range of from about 0.02 to 0.1% of bone marrow cells.

Once hematopoietic stem cells have been isolated, they may be propagated by growing in conditioned medium from stromal cells, such as those that can be obtained from bone marrow or liver associated with the secretion of factors, or in medium comprising cell surface factors supporting the proliferation of hematopoietic stem cells. Stromal cells may be freed of hematopoietic cells employing appropriate monoclonal antibodies for removal of the undesired cells, for example, with antibody-toxin conjugates, antibody and complement, etc.

The hematopoietic cell compositions may find use in a variety of ways. Since the cells are naive, they can be used to fully reconstitute a lethally irradiated host, such a patient undergoing cancer therapy, and can be used as a source of cells for specific lineages, by providing for their maturation, proliferation, and differentiation into one or more selected lineages by employing a variety of factors, such as erythropoietin, GM-CSF, G-CSF, M-CSF, interleukins, e.g. IL-1, -2, -3, -4, -5, -6, -7, etc., or the like, or stromal cells associated with the hematopoietic stem cells becoming committed to a particular lineage, or with their proliferation, maturation and differentiation.

The hematopoietic stem cells may also be used in the isolation and evaluation of factors associated with the differentiation and maturation of hematopoietic cells. Thus, the hematopoietic stem cells may be used in assays to determine the activity of media, such as conditioned media, evaluate fluids for cell growth activity, involvement with dedication to particular lineages, or the like.

The cells may be frozen at liquid nitrogen temperatures and stored for long periods of time, being thawed and capable of being reused. The cells will usually be stored in 10% DMSO, 50% FCS, 40% RPMI 1640 medium. Once thawed, the cells may be expanded by use of growth factors and/or stromal cells associated with hematopoietic stem cell proliferation and differentiation, which are known the in the art.

Relevant publications include the following:
Bodger, M. P. et al. (1981) “A monoclonal antibody specific for immature human hemopoietic cells and T lineage cells” Journal of Immunology 127(6):2269-2274.
Ritz, J. et al. (1980) “A monoclonal antibody to human acute lymphoblastic leukaemia antigen” Nature 283:583-585.
Civin, C. I. et al. (1984) “Antigenic Analysis of Hematopoiesis III. A Hematopoietic Progenitor Cell Surface Antigen Defined by a Monoclonal Antibody Raised against KG-1a Cells” Journal of Immunology 133(1):157-165.
Craig, W. et al. (1993) “Expression of Thy-1 on Human Hematopoietic Progenitor Cells” J. Ex. Med. 177:1331-1342.
Berenson, R. J. et al. (1988) “Antigen CD34.sup.+ Marrow Cells Engraft Lethally Irradiated Baboons” J. Clin. Invest. 81:951-955.
Terstappen, L. W. (1990) “Flow Cytometric Analysis of Human Bone Marrow III. Neutrophil Maturation” Leukemia 4(9):657-663.
Loken, M. R. et al. (1987) “Flow Cytometric Analysis of Human Bone Marrow II. Normal B. Lymphocyte Development” Blood 70(5):1316-1324.
Friedmann, T. (1989) “Progress Toward Human Gene Therapy” Science 244:1275-1281.
Sutherland, H. J. et al. (1991) “Differential Regulation of Primitive Human Hematopoietic Cells in Long-Term Cultures Maintained on Genetically Engineered Murine Stromal Cells” Blood 78(3):666-672.
Terstappen, L. W. et al. (1991) “Sequential Generations of Hematopoietic Colonies Derived From Single Nonlineage-Committed CD34+CD38-Progenitor Cells” Blood 77(6):1218-1227.
Bender, J. G. et al. (1991) “Identification and Comparison of CD34-Positive Cells and Their Subpopulations From Normal Peripheral Blood and Bone Marrow Using Multicolor Flow Cytometry” Blood 77(12):2591-2596.
Verfaillie, C. et al. (1990) “Purified Primitive Human Hematopoietic Progenitor Cells with Long-Term In Vitro Repopulating Capacity Adhere Selectively to Irradiated Bone Marrow Stroma” J. Exp. Med. 172:509-520.
Loken, M. R. et al. (1987) “Flow Cytometric Analysis of Human Bone Marrow: I. Normal Erythroid Development” Blood 69(1):255-263.
Simmons, P. J. et al. (1991) “Identification of Stromal Cell Precursors in Human Bone Marrow by a Novel Monoclonal Antibody, STRO-1” Blood 78(1):55-62.
Golde, D. W. (1991) “The Stem Cell” Scientific American 265(6):86-93. The Lancet (1988) Gene Therapy in Man. 1:1271-1272.

### Example 5

### Cancer Diagnosis and Prognosis

The present invention provides novel methods for diagnosis and prognostic evaluation of cancer (e.g., colon cancer, lung cancer, breast cancer, head and neck cancer). In one aspect, the expression levels of genes are determined in different patient samples for which either diagnosis or prognosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is unique to the state of the cell. That is, normal tissue may be distinguished from a cancer tissue, and within a cancer tissue, different prognosis states (good or poor long term survival prospects, for example) may be determined. By comparing expression profiles of cancer tissue in different states, information regarding which genes are important (including both up- and down-regulation of genes) in each of these states is obtained. The identification of sequences that are differentially expressed in cancer tissue versus normal tissue of that type (e.g., breast cancer tissue versus normal breast tissue), as well as differential expression resulting in different prognostic outcomes, allows the use of this information in a number of ways. For example, the evaluation of a particular treatment regime may be evaluated: does a chemotherapeutic drug act to improve the long-term prognosis in a particular patient Similarly, diagnosis may be done or confirmed by comparing patient samples with the known expression profiles. See, e.g., U.S. Pat. No. 6,780,586.

Furthermore, these gene expression profiles (or individual genes) allow screening of drug candidates with an eye to mimicking or altering a particular expression profile; for example, screening can be done for drugs that suppress the cancer expression profile or convert a poor prognosis profile to a better prognosis profile. This may be done by making biochips comprising sets of the important cancer genes, which can then be used in these screens. These methods can also be done on the protein basis; that is, protein expression levels of the cancer proteins can be evaluated for diagnostic and prognostic purposes (using, e.g., hLy6-BIG antibodies) or to screen candidate agents. In addition, the hLy6-BIG nucleic acid sequences can be administered for gene therapy purposes, including the administration of antisense nucleic acids, or the hLy6-BIG antibodies administered as therapeutic drugs.

hLy6-BIG (e.g., hLy6-BIG1) molecules find use as markers of cancer (e.g. breast cancer, head and neck cancer) (see Example 7 for additional cancers). Detection of molecules in putative cancer tissue of patients allows for a determination or diagnosis of the cancer. Numerous methods known to those of ordinary skill in the art find use in detecting cancer. In one embodiment, antibodies are used to detect cancer proteins. A preferred method separates proteins from a sample or patient by electrophoresis on a gel (typically a denaturing and reducing protein gel, but may be any other type of gel including isoelectric focusing gels and the like). Following separation of proteins, hLy6-BIG is detected by immunoblotting with antibodies. Methods of immunoblotting are well known to those of ordinary skill in the art.

In another preferred method, antibodies to hLy6-BIG find use in in situ imaging techniques. In this method cells are contacted with from one to many hLy6-BIG antibodies. Following washing to remove nonspecific antibody, binding, the presence of the hLy6-BIG antibody or antibodies is detected. In one embodiment the antibody is detected by incubating with a secondary antibody that contains a detectable label. In another method the primary antibody to hLy6-BIG contains a detectable label. In another preferred embodiment each one of multiple primary antibodies contains a distinct and detectable label. This method finds particular use in simultaneous screening for a plurality of cancer proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In a preferred embodiment, in situ hybridization of labeled hLy6-BIG nucleic acid probes to tissue arrays is done. For example, arrays of tissue samples, including cancer tissue and/or normal tissue, are made. In situ hybridization as is known in the art can then be done.

It is understood that when comparing the fingerprints between an individual and a standard, the skilled artisan can make a diagnosis as well as a prognosis.

In a preferred embodiment, the hLy6-BIG proteins, antibodies, nucleic acids, and cells containing hLy6-BIG sequences are used in prognosis assays. As above, gene expression profiles can be generated that correlate to cancer severity, in terms of long term prognosis. Again, this may be done on either a protein or gene level, with the use of genes being preferred. As above, the hLy6-BIG probes are attached to biochips for the detection and quantification of hLy6-BIG sequences in a tissue or patient The assays proceed as outlined for diagnosis.

In a preferred embodiment, hLy6-BIG molecules are used in drug screening assays or by evaluating the effect of drug candidates on a gene expression profile or expression profile of polypeptides. In a preferred embodiment, the expression profiles are used, preferably in conjunction with high throughput screening techniques to allow monitoring for expression profile genes after treatment with a candidate agent, Zlokarnik, et al., Science 279, 84-8 (1998), Heid, Genome Res. 6:986-94 (1996).

In a preferred embodiment, the hLy6-BIG molecules are used in screening assays for compositions which modulate the cancer phenotype. As above, this can be done on an individual gene level or by evaluating the effect of drug candidates on a “gene expression profile”. In a preferred embodiment, expression profiles are used, preferably in conjunction with high throughput screening techniques to allow monitoring for expression profile genes after treatment with a candidate agent, see Zlokarnik, supra.

### Example 6

### Cancer Therapy

The method of the present invention may be used to prevent progression to a neoplastic or malignant state, including but not limited to those disorders described herein. In preferred embodiments, the method of the invention is used to inhibit growth, progression, and/or metastasis of cancers, e.g., those listed herein.

The hLy6-BIG molecules may be administered in either a single dose or multiple doses at different time points in the therapy or treatment regimen.

The hLy6-BIG molecules of the invention can be administered alone or co-administered with a chemotherapeutic agent and/or radiation therapy, or administered separately, before, during or after chemotherapeutic administration or radiation therapy.

Chemotherapeutic agents can be administered at known concentrations according to known techniques. Exemplary chemotherapeutic agents include alkylating agents such as nitrogen mustards, ethylenimines, methylmelamines, alkyl sulfonates, nitrosuoureas, and triazenes; antimetabolites such as folic acid analogs, pyrimidine analogs, in particular fluorouracil and cytosine arabinoside, and purine analogs; natural products such as vinca alkaloids, epipodophyllotoxins, antibiotics, enzymes and biological response modifiers; and miscellaneous products such as platinum coordination complexes, anthracenedione, substituted urea such as hydroxyurea, methyl hydrazine derivatives, and adrenocorticoid suppressant.

Exemplary chemotherapeutic agents also include vinca alkaloids, epipodophyllotoxins, anthracycline antibiotics, actinomycin D, plicamycin, puromycin, gramicidin D, paclitaxel (Taxol®, Bristol Myers Squibb), colchicine, cytochalasin B, emetine, maytansine, and amsacrine (or “mAMSA”). The vinca alkaloid class is described in Goodman and Gilman's The Pharmacological Basis of Therapeutics (7th ed.), (1985), pp. 1277-1280. Exemplary of vinca alkaloids are vincristine, vinblastine, and vindesine. The epipodophyllotoxin class is described in Goodman and Gilman's The Pharmacological Basis of Therapeutics (7th ed), (1985), pp. 1280-1281. Exemplary of epipodophyllotoxins are etoposide, etoposide orthoquinone, and teniposide. The anthracycline antibiotic class is described in Goodman and Gilman's The Pharmacological Basis of Therapeutics (7th ed.), (1985), pp. 1283-1285. Exemplary of anthracycline antibiotics are daunorubicin, doxorubicin, mitoxantraone, and bisanthrene. Actinomycin D, also called Dactinomycin, is described in Goodmand and Gilman's The Pharmacological Basis of Therapeutics (7th ed.), (1985), pp. 1281-1283. Plicamycin, also called mithramycin, is described in Goodmand and Gilman's The Pharmacological Basis of Therapeutics (7th ed.), (1985), pp. 1287-1288. Additional chemotherapeutic agents include cisplatin (Platinol®, Bristol Myers Squibb), carboplatin (Paraplatin®, Bristol Myers Squibb), mitomycin (Mutamycin®, Bristol Myers Squibb), altretamine (Hexylen®, U.S. Bioscience, Inc.), cyclophosphamide (Cytoxan®, Bristol Myers Squibb), lomustine (CCNU) (CeeNU®, Bristol Myers Squibb), carmustine (BCNU) (BiCNU®, Bristol Myers Squibb).

Additional therapeutic agents which may be administered in combination with Ly6-BIG molecules of the invention also include aclacinomycin A, aclarubicin, acronine, acronycine, adriamycin, aldesleukin (interleukin-2), altretamine (hexamethylmelamine), aminoglutethimide, aminoglutethimide (cytadren), aminoimidazole carboxamide, amsacrine (m-AMSA; amsidine), anastrazole (arimidex), ancitabine, anthracyline, anthramycin, asparaginase (elspar), azacitdine, azacitidine (ladakamycin), azaguanine, azaserine, azauridine, 1,1′,1″-phosphinothioylidynetris aziridine, azirino(2′,3′:3,4)pyrrolo[1,2-a]indole-4,7-dione, BCG (theracys), BCNU, BCNU chloroethyl nitrosoureas, benzamide, 4-(bis(2-chloroethyl)amino)benzenebutanoic acid, bicalutamide, bischloroethyl nitrosourea, bleomycin, bleomycin (blenozane), bleomycins, bromodeoxyuridine, broxuridine, busulfan (myleran), carbamic acid ethyl ester, carboplatin, carboplatin (paraplatin), carmustine, carmustine (BCNU; BiCNU), chlorambucil (leukeran), chloroethyl nitrosoureas, chorozotocin (DCNU), chromomycin A3, cis-retinoic acid, cisplatin (cis-ddpl; platinol), cladribine (2-chlorodeoxyadenosine; 2cda; leustatin), coformycin, cycloleucine, cyclophosphamide, cyclophosphamide anhydrous, chlorambucil, cytarabine, cytarabine, cytarabine HCl (cytosar-u), 2-deoxy-2-(((methylnitrosoamino)carbonyl)amino)-D-glucose, dacarbazine, dactinomycin (cosmegen), daunorubicin, Daunorubincin HCl (cerubidine), decarbazine, decarbazine (DTIC-dome), demecolcine, dexamethasone, dianhydrogalactitol, diazooxonorleucine, diethylstilbestrol, docetaxel (taxotere), doxorubicin HCl (adriamycin), doxorubicin hydrochloride, eflornithine, estramustine, estramustine phosphate sodium (emcyt), ethiodized oil, ethoglucid, ethyl carbamate, ethyl methanesulfonate, etoposide (VP16-213), fenretinide, floxuridine, floxuridine (fudr), fludarabine (fludara), fluorouracil (5-FU), fluoxymesterone (halotestin), flutamide, flutamide (eulexin), fluxuridine, gallium nitrate (granite), gemcitabine (gemzar), genistein, 2-deoxy-2-(3-methyl-3-nitrosoureido)-D-glucopyranose, goserelin (zoladex), hexestrol, hydroxyurea (hydra), idarubicin (idamycin), ifosfagemcitabine, ifosfamide (iflex), ifosfamide with mesna (MAID), interferon, interferon alfa, interferon alfa-2a, alfa-2b, alfa-n3, interleukin-2, iobenguane, iobenguane iobenguane, irinotecan (camptosar), isotretinoin (accutane), ketoconazole, 4-(bis(2-chloroethyl)amino)-L-phenylalanine, L-serine diazoacetate, lentinan, leucovorin, leuprolide acetate (LHRH-analog), levamisole (ergamisol), lomustine (CCNU; cee-NU), mannomustine, maytansine, mechlorethamine, mechlorethamine HCl (nitrogen mustard), medroxyprogesterone acetate (provera, depo provera), megestrol acetate (menace), melengestrol acetate, melphalan (alkeran), menogaril, mercaptopurin, mercaptopurine (purinethol), mercaptopurine anhydrous, MESNA, mesna (mesne), methanesulfonic acid, ethyl ester, methotrexate (mtx; methotrexate), methyl-ccnu, mimosine, misonidazole, mithramycin, mitoantrone, mitobronitol, mitoguazone, mitolactol, mitomycin (mutamycin), mitomycin C, mitotane (o,p′-DDD; lysodren), mitoxantrone, mitoxantrone HCl (novantrone), mopidamol, N,N-bis(2-chloroethyl)tetrahydro-2H-1,3,2-oxazaphosphorin-2-amine-2-oxide, N-(1-methylethyl)-4-((2-methylhydrazino)methyl)benzamide, N-methyl-bis(2-chloroethyl)amine, nicardipine, nilutamide (nilandron), nimustine, nitracrine, nitrogen mustard, nocodazole, nogalamycin, octreotide (sandostatin), pacilataxel (taxon), paclitaxel, pactamycin, pegaspargase (PEGx-1), pentostatin (2% deoxycoformycin), peplomycin, peptichemio, photophoresis, picamycin (mithracin), picibanil, pipobroman, plicamycin, podofilox, podophyllotoxin, porfiromycin, prednisone, procarbazine, procarbazine HCl (matulane), prospidium, puromycin, puromycin aminonucleoside, PUVA (psoralen +ultraviolet a), pyran copolymer, rapamycin, s-azacytidine, 2,4,6-tris(1-aziridinyl)-s-triazine, semustine, showdomycin, sirolimus, streptozocin (zanosar), suramin, tamoxifen citrate (nolvadex), taxon, tegafur, teniposide (VM-26; vumon), tenuazonic acid, TEPA, testolactone, thio-tepa, thioguanine, thiotepa (thioplex), tilorone, topotecan, tretinoin (vesanoid), triaziquone, trichodermin, triethylene glycol diglycidyl ether, triethylenemelamine, triethylenephosphoramide, triethylenethiophosphoramide, trimetrexate (neutrexin), tris(1-aziridinyl)phosphine oxide, tris(1-aziridinyl)phosphine sulfide, tris(aziridinyl)-p-benzoquinone, tris(aziridinyl)phosphine sulfide, uracil mustard, vidarabine, vidarabine phosphate, vinblastine, vinblastine sulfate (velban), vincristine sulfate (oncovin), vindesine, vinorelbine, vinorelbine tartrate (navelbine), (1)-mimosine, 1-(2-chloroethyl)-3-(4-methylcyclohexyl)-1-nitrosourea, (8S-cis)-10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-5,12-naphthacenedione, 13′-meta-iodobenzyl guanidine (1-131 MIBG), 5-(3,3-dimethyl-1-triazenyl)-1H-imidazole-4-carboxamide, 5-(bis(2-chloroethyl)amino)-2,4(1H,3H)-pyrimidinedione, 2,4,6-tris(1-aziridinyl)-s-thiazine, 2,3,5-tris(1-aziridinyl)-2,5-cyclohexadiene-1,4-dione, 2-chloro-N-(2-chloroethyl)-N-methylethanamine, N,N-bis(2-chloroethyl)tetrahydro-2H-1,3,2-oxazaphosphorin-2-amine-2-oxide, 3-deazauridine, 3-iodobenzylguanidine, 5,12-naphthacenedione, 5-azacytidine, 5-fluorouracil, (1aS,8S,8aR,8bS)-6-amino-8-(((aminocarbonyl)oxy)methyl)-1,1a,2,8,8a,8b-hexahydro-8a-methoxy-5-methylazirino(2′,3′:3,4)pyrrolo[1,2-a]indole-4,7-dione, 6-azauridine, 6-mercaptopurine, 8-azaguanine, and combinations thereof.

### Example 7

### Types of Cancer

Examples of cancer that may be treated, diagnosed, prognosed, or prevented include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancer include neoplasms located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Other examples of cancer include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

Additional diseases or conditions associated with increased cell survival that could be treated by the method of the invention, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

Similarly, other cancers can also be treated by the method of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above.

### Example 8

### Ly6-BIG Expression

### Construction of Full Length LY6-BIG1

A DNA fragment encoding the predicted open reading frame region of human LY6-BIG1 was amplified by PCR from human spleen cDNA (Clontech) (FIG. 1). Primers used for amplifying LY6-BIG1 were: PCf1 forward primer 5′ CTGAAGTTTGTCTGTGCACTAG 3′ (SEQ ID NO:______) and PCr1 reverse primer 5′ GCAGCTCTTCAAAACCAAGCAG 3′ (SEQ ID NO:______) (FIG. 1). PCR products of the correct size were isolated, cloned into the cloning vector pCR4-TOPO (Invitrogen), and sequenced. A clone whose sequence entirely matched the predicted LY6-BIG1 sequence was used for construction of the soluble Fc fusion protein.

### Construction of Soluble Ly6-BIG1 Fc Fusion Protein

To construct a soluble form of LY6-BIG1 protein we designed two chimeric proteins consisting of the extracellular domain of human LY6-BIG1 fused to a human IgG1 Fc domain. The native LY6-BIG1 leader sequence (predicted to consist of amino acid residues 1-26) was retained for directing secretion (Table 20). Human LY6-BIG1 has C-terminal sequences that suggest the presence of a potential GPI-modification site. GPI-modified proteins have a C-terminal propeptide that is proteolytically removed followed by attachment of a GPI moiety. The GPI-modified protein is subsequently anchored in the plasma membrane. The site of GPI attachment at the carboxyl terminus is referred to as the omega-site. In order to design a LY6-BIG1 Fc fusion protein that most faithfully reflects the native LY6-BIG1 extracellular structure it is necessary to identify the putative omega-site residue. A primary potential omega-site residue was identified at serine 112 (FIG. 2) using the big-PI Predictor GPI Modification Site Prediction algorithm (The GPI Prediction Server Version 1.5, References 1-4). Subsequently, a LY6-BIG1 Fc fusion protein, LY6-BIG1 Fc1 (Table 20), was designed consisting of LY6-BIG1 amino acid residues 1-112 fused to a human IgG1 Fc domain. Because the extracellular domain of LY6-BIG1 Fc1 fusion protein contains an uneven number of cysteine residues, in contrast to the murine homologue, we were concerned that a single unpaired cysteine might possibly compromise correct protein folding. Therefore we also designed a second fusion protein, LY6-BIG1 Fc2 (Table 21), consisting of LY6-BIG1 amino acid residues 1-126 that included the downstream cysteine residue at position 118 as a fusion to the human IgG1 Fc domain.

Construction of the LY6-BIG1 Fc fusion protein required generation of the appropriate fragments of the LY6-BIG1 coding region for insertion into a mammalian expression vector containing human IgG1 Fc cDNA sequence. PCR primers used to amplify LY6-BIG1 amino acid residues 1-112 (for LY6-BIG1 Fc 1) were:

SCFC 1 forward primer 5′ACTAGCGGATCCCTCACCATGGGCAGTCTCCAGGCCATGAAGAC 3′ (SEQ ID NO:______) and SCFC 2 reverse primer 5′ CTCCTGGCTAGCGCTGGCTGCCAGGACCACCG 3′ (SEQ ID NO:______). PCR amplification of LY6-BIG1 amino acid residues 1-126 used the same forward primer, SCFC, and SCFC 3 reverse primer 5′ CTCCTGGCTAGCCCCCAGGCTGAGCAGGAGCTGTA 3′ (SEQ ID NO:______).

The PCR products were isolated and the amino and carboxyl terminal DNA fragments digested with Bam HI and Nhe I, respectively. The proprietary mammalian expression vector was digested with Bg1II and NheI to create a proper insertion site for the LY6-BIG1 extracellular domain upstream of the Fc sequence, and ligated to each of the PCR-generated fragments to complete the two fusion protein constructs. DNA sequencing confirmed that the fusion sequences were correct.

### LY6-BIG1 Expression

The two LY6-BIG1 Fc fusion protein constructs (Fc1 and Fc2) were subsequently used for transient transfection of the COS7 cell line. Briefly, expression constructs were transfected into COS7 cells using Lipofectamine (Invitrogen). After 72 hours culture, the culture supernatants were collected, filtered, and analyzed for secreted LY6-BIG1 Fc fusion protein by ELISA, using a different Fc fusion protein as the standard. The calculated concentration the secreted LY6-BIG1 Fc fusion protein was 3 ug/ml. Incubation with Protein A beads (Zymed) was used to purify Fc fusion proteins from the supernatants, and this material was assayed by immunoblot. 15 uL of supernatant from Fc1, Fc2 and mock transfectants were also analyzed in the same immunoblot. Briefly, samples were prepared in 4×LDS Sample Buffer (Invitrogen) containing 2-mercaptoethanol and resolved on a 4-20% NuPage SDS gel. CTLA-4 Ig, a fusion protein made from the same Fc vector was used for comparison on the gel. The proteins were then transferred to a PVDF membrane. The membrane was blocked with 5% non-fat milk in western buffer (TPBS/0.001% Tween-20) overnight at 4□C, then probed with a horseradish peroxidase (HRP) conjugated goat anti-human IgG (Southern Biotechnology) at a 1:10,000 dilution for 1 hour at 4° C. with gentle rocking. Following 5 washes in western buffer, the membrane was developed using ECL reagent (Amersham Pharmacia) according to manufacturer's instructions. A band of approximately 46 kDa (arrow), consistent with the predicted MW of the Ly6-BIG1 Fc fusion protein, was seen (FIG. 11). Higher molecular weight bands likely represent aggregates or dimers of the Fc fusion protein. This data indicates that recombinant LY6-BIG1 molecules can be expressed successfully at high levels in mammalian cells.

### REFERENCES

1. Eisenhaber B., Bork P., Eisenhaber F. “Sequence properties of GPI-anchored proteins near the omega-site: constraints for the polypeptide binding site of the putative transamidase” Protein Engineering (1998) 11, No. 12, 1155-1161.
2. Sunyaev S. R., Eisenhaber F., Rodchenkov I. V., Eisenhaber B., Tumanyan V. G., and Kuznetsov E. N. “Prediction of potential GPI-modification sites in proprotein sequences” Protein Engineering (1999) 12, No. 5, 387-394.
3. Eisenhaber B., Bork P., Eisenhaber F. “Prediction of potential GPI-modification sites in proprotein sequences” JMB (1999) 292 (3), 741-758.
4. Eisenhaber B., Bork P., Yuan Y., Loeffler G., Eisenhaber F. “Automated annotation of GPI anchor sites: case study C.elegans” TIBS (2000) 25 (7), 340-341.

### Example 9

### PCR Protocol for Ly6-BIG 1-7 mRNA Detection

For PCR amplification experiments, cDNA was amplified with Platinum Taq DNA polymerase (Invitrogen) and its accompanying 10×PCR buffer for 32 cycles: melting at 94□C for 30 sec, annealing at 58□C for 30 sec, extending at 72□C for 30 sec. Final concentrations in the reaction were: 1×PCR buffer, 1.5 mM MgCl2, 0.2 mM dNTPs, 0.2 μM forward and reverse PCR primers, 1 unit polymerase in a 25 ul reaction, along with 1-2.5 μl of cDNA. Spleen and brain cDNAs and fetal brain RNA were purchased from Clontech. Bone marrow mononuclear cell RNA was purchased from Allcells. RNAs were converted to cDNAs using Super Script First Strand Synthesis System from Invitrogen.

Cloning of PCR products for sequence analysis was performed using TOPO TA Cloning Kit for Sequencing (Invitrogen) and XL1-Blue Competent Cells (Stratagene).

PCR Primers

| | |
|---|---|
| BIG1 - PCf2 | 5′ CAT GAA GAC CTT GTC CCT GGT CCT G |
| BIG1 - 395r | 5′ GCC CAG AGG AAG ACT GAC CC |
| BIG2 - 8f | 5′ GGC TGC TGG TGG CGT TGC |
| BIG2 - 567r | 5′ CTC GTT GTA GGA GAT CCA GTG CTG C |
| BIG2 - 612r | 5′ GAC ATC ACT GAG CCC ACT CTC C |
| BIG3 - 35f | 5′ TGC TGG CTG CCT GCG GAG AG |
| BIG3 - 363r | 5′ CAA GAG TGG GAG GAG CGT GAG G |
| BIG3 - 375r | 5′ CAG TCG GAG GCT CAA GAG TGG |
| BIG4 - 7f | 5′ CTT CTC CTG GTG CTG CTG CTT C |
| BIG4 - 25f | 5′ CTT CTG GCT GCT GTG TGT GCT G |
| BIG4 - 334r | 5′ GCT GGC TGG CTG CGA TGC TG |
| BIG5 - 3f | 5′ GAA GGT CAC TAG CCC CAT GCT GC |
| BIG5 - 17f | 5′ CCA TGC TGC TGC TGG CTG AG |
| BIG5 - 358r | 5′ GGC TAT TTT CAC AGC AGG TCA TGT C |
| BIG6 - 6f | 5′ GAG GCT CGT CCT AAC CCT GTG C |
| BIG6 - 41f | 5′ CTG TGG CGT CTG CTG GCT GC |
| BIG6 - 959r | 5′ CCG TCC ACA CTG GCA AGG AAC |
| BIG7 - 25f | 5′ CTT GCC CTC TTG CTG TGC GG |
| BIG7 - 365r | 5′ CCC ACA GTT TCG GAG CTG CC |
| BIG7 - 394r | 5′ CGG CAA GGA GGT TGA GCA GG |

### Results Summary for BIG 1-7 PCR

We found evidence of transcription of three of the novel genes in RT-PCR experiments: BIG1, BIG3, and BIG7 (Table 23). BIG1 was found in spleen and brain, and some tumor cell lines, sometimes as an alternate splice variant. BIG3 mRNA was detected in bone marrow mononuclear cells and in spleen. BIG7 mRNA was found in brain and spleen; however, sequence data from the spleen PCR product revealed the presence of an extra exon.

### Example 10

### Production and Testing of Anti-Ly6-BIG1 Hybridomas

Five female Balb/c mice were each immunized s.c. with 4 ug BIG1-Ig in Freund's Complete Adjuvant (FCA). Three weeks later, each mouse was again immunized s.c with 4 ug BIG1-Ig in Freund's Incomplete Adjuvant (FICA). Bleeds were taken 10 days after each boost to assess animal response by ELISA. Four weeks after the second boost three of the mice were each immunized i.v. with 4 ug BIG1-Ig in PBS.

NS-1 myeloma cells (HAT sensitive) were used as the fusion partner, and were kept in exponential growth phase for one week prior to fusion. Ideally, 50×10e6 NS-1 cells are used for each mouse spleen.

The fusion protocol was performed as follows: Three days after the i.v. boost, spleens from the three mice were harvested aseptically in wash buffer (WB; RPMI 1640 medium). Single cell suspensions were prepared by teasing cells apart and flushing them through a filter into a 50 ml tube. The HAT sensitive NS-1 myelomas were harvested into 50 ml or 250 ml tubes. The PEG 1500 (Roche 783 641), WB, and HAT medium were all brought to 37° C. Both cell populations (spleen and NS-1 myeloma) were centrifuged at 1500 rpm for 7 minutes, and the supernatants were aspirated. The RBCs were lysed from the splenocytes with 7 ml Lysing Buffer for 1 minute, and the volume was then brought to 40 ml with WB. The myeloma cells were resuspended in 40 ml WB.

The splenocytes and myeloma cells were centrifuged at 1500 rpm for 5 minutes, and the supernatants were aspirated. Each cell type was resuspended in 40 mls and again centrifuged at 1500 rpm for 5 minutes. This wash step was repeated one more time, and each cell type was resuspended in 25 mls WB. The splenocytes and myeloma cells were counted and viability was determined for each. The myeloma cells were added to the splenocytes in a 1:4 ratio (myeloma:splenocyte), and 2.4×10e6 myeloma cells were set aside and resuspended in HAT medium (Iscoves Modified Dulbeccos Medium (Irvine Scientific #9032) supplemented with 10% Fetal Bovine Serum (heat inactivated), L-Glutamine (Gibco #25030-081), NEAA (Sigma #M7145), Na Pyruvate (Sigma # S8636), Gentamicin (Gibco #15750-060), 10% Hybridoma Cloning Factor (Bioveris #210001) and HAT supplement 50× (Cellgro #25-046-C1)) to plate as a control plate. The mixed cells were centrifuged for 7 minutes and the sup ematants were aspirated completely. The tubes were tapped on the on hood to spread out the pellets. One ml of 50% PEG was added over 1 minute while tapping and stirring gently and then 1 ml of WB was added over 1 minute while tapping and stirring gently. Eight mls of WB was added over 2 minutes while tapping and stirring gently. The cells were then centrifuged at 900 rpm for 10 minutes. The supernatants were aspirated and the cells were resuspended gently by pipetting HAT medium onto the pellet (# of splenos/70,000)×100 ul HAT medium), the cell chunks were allowed to settle, and the resuspended cells were added to the HAT bottle repeatedly until the chunks were dissolved while keeping sheering forces to a minimum. Splenocytes should be at 1×10e6 per ml in HAT. 100 ul of cells were aliquoted to each well of a 3595 TC plate using wide orifice tips. The plate was placed in 37° C. incubator. After 3 days, 100 ul of HT (Iscoves medium, 5% Hybridoma Cloning Factor, and HT supplement 50× (Cellgro #25-047-C1)) was added to each well. The wells were cut and fed as needed (the medium was removed and replaced with fresh medium). The fusions were screened between days 7 and 12 and the positive wells were expanded and cloned.

Fusions were screened by ELISA 10 days later on both BIG1-Ig and CTLA4-Ig (negative control). The protocol for the ELISA was as follows: Plates (96 well round bottom plates from Immulon2 HP VWR cat#62402-954) were coated with 50 ul/well antigen (2 ug/ml antigen/capture in carb/bicarb 019.6) overnight at 4° C. Plates were washed in PBS, 0.025% Tween20 using Skatron RB#1 program. Plates were blocked with 250 ul/well Block Buffer (PBS, 2% non-fat milk, 0.025% Tween20) for at least 1 hour at room temperature. Plates were flicked and banged to completely remove the Block Buffer. 50 ul of diluted hybridoma supernatant (1:1 in Block Buffer) was added to each well and the plates were incubated 1 hour at room temperature. The plates were washed using Skatron RB#2 program. 50 ul od secondary Ab (a pool of goat anti-mouse kappa HRP (Southern Biotech 1050-05) at 1:2000 and goat anti-mouse lambda HRP (Southern Biotech 1060-05) at 1:2000 was added to each well, and the plates were incubated for 1 hour at room temperature. The plates were washed using Skatron RB#2 program and banged dry. 60 ul of premixed substrate (3,3′,5,5′-tetramethylbenzidine) was added and allowed to develop for 3 minutes. The reaction was quenched with 60 ul of 4N sulfuric acid and the plates were read at 450 nm with a 750 nm reference.

Positives for BIG1-Ig and negative for CTLA4-Ig were expanded and subcloned twice, by limiting dilution. After subcloning, hybridoma supernatants were again tested against BIG1-Ig and a panel of negative Ig fusion proteins (CTLA4-Ig, Cripto-Ig, B7-1-Ig, and LTBeta) to ensure specificity and no cross-reactivity. The positives showed strong binding to BIG1-Ig and no activity on the negative fusion proteins.

Furthermore, supernatants were tested by flow cytometry on BIG1 transfected CHO cells and found to be positive on the transfected cells and negative on the untransfected control cells (FIG. 13) The protocol for flow cytometry was as follows: All steps were performed on ice if possible. All dilutions were done in FACS buffer (D-PBS, 2% FBS, 0.05% sodium azide & 10% Normal Goat Serum (heat inactivated)). 50 ul of 2× primary mAbs was aliquoted to wells in Corning 3799 plate(s). Cells were harvested by centrifugation at 1500 rpm for 7 minutes and resuspension in FACS buffer. Cells were ideally adjusted to 2−20×10e6/ml. Cells were kept on ice in sodium azide for at least 15 minutes, preferably 45. 50 ul of cells was added to the primary antibodies (1×10e5 to 1×10e6), mixed, and incubated on ice for 45 minutes. 50 ul of FACS buffer was added to each well and the plates were spun for 4 minutes at 1500 rpm. The supernatants were needle aspirated and the cells were resuspended in 150 ul of FACS buffer. The wash step was repeated. 100 ul of 1× rat anti-mouse kappa-biotin at 1:500 (Southern Biotech 1170-08) was added and mixed. The mixture was incubate for 45 minutes on ice. 100 ul of 1× Strepavidin-APC at 1:500 (Pharmingen)) was added and mixed. The mixture was incubated for 45 minutes on ice. 50 ul of FACS buffer (with 7-aminoactinomycin D at 1:300 for 1:900 final of a 1 mg/ml stock solution) was added. The plates were spun and the supernatants removed by aspiration. The cells were resuspended in 150 ul of FACS buffer, spun, and aspirated. The cells were resuspended in 100 ul of FACS buffer. The cells were transferred to fresh 12×75 mm tubes with 200 ul FACS buffer. Readings were taken on a FACS machine.

The hybridoma subclones were isotyped using IsoStrip (Roche). We found that all were IgG1 kappa with the exception of 22G4, which was IgM kappa.

Western blot analysis showed that two of the hybridoma antibodies specifically bound BIG-1 protein in lysates from CHO-BIG1 transfectants (FIGS. 13 and 14).

All publications such as textbooks, journal articles, GenBank entries, patents, published applications, and all patent applications mentioned in this specification are herein incorporated by reference to the same extent as if each individual publication or patent application was specifically and individually indicated to be incorporated by reference.

TABLE 1
SEQ ID NO: 1
>ly6-BIG1.1_DNA
ATGAGCAGTCTCCAGGCCATGAAGACCTTGTCCCTGGTCCTGCTGGTGG
CCCTGCTGAGCATGGAGAGAGCTCAGGGTCTGCGCTGCTACAGATGCTT
GGCGGTCTTGGAAGGGGCCTCCTGCAGCGTGGTCTCGTGCCCCTTCCTG
GATGGGGTCTGTGTCTCCCAGAAAGTGAGCGTCTTTGGCAGTAAAGTGA
GAGGGGAGAACAAGCTCTCCCTCCTCTCCTGCCAGAAGGACGTCGGATT
CCCCCTGCTGAAACTTACAAGTGCCGTTGTGGACTCCCAGATCTCTTGC
TGCAAGGGAGACCTCTGCAATGCGGTGGTCCTGGCAGCCAGCAGCCCCT
GGGCCCTGTGCGTACAGCTCCTGCTCAGCCTGGGGTCAGTCTTCCTCTG
GGCCCTGCTGTGA
SEQ ID NO: 2
>ly6-BIG1.1_Protein
MSSLQAMKTLSLVLLVALLSMERAQGLRCYRCLAVLEGASCSVVSCPFL
DGVCVSQKVSVFGSKVRGENKLSLLSCQKDVGFPLLKLTSAVVDSQISC
CKGDLCNAVVLAASSPWALCVQLLLSLGSVFLWALL

TABLE 2
SEQ ID NO: 3
>ly6-BIG1.2_DNA
ATGAGCAGTCTCCAGGCCATGAAGACCTTGTCCCTGGTCCTGCTGGTG
GCCCTGCTGAGCATGGAGAGAGCTCAGGGTCTGCGCTGCTACAGATGC
TTGGCGGTCTTGGAAGGGGCCTCCTGCAGCGTGGTCTCGTGCCCCTTC
CTGGATGGGGTCTGTGTCTCCCAGAAAAAGGACGTCGGATTCCCCCTG
CTGAAACTTACAAGTGCCGTTGTGGACTCCCAGATCTCTTGCTGCAAG
GGAGACCTCTGCAATGCGGTGGTCCTGGCAGCCAGCAGCCCCTGGGCC
CTGTGCGTACAGCTCCTGCTCAGCCTGGGGTCAGTCTTCCTCTGGGCC
CTGCTGTGA
SEQ ID NO: 4
>ly6-BIG1.2_Protein
MSSLQAMKTLSLVLLVALLSMERAQGLRCYRCLAVLEGASCSVVSCPF
LDGVCVSQKKDVGFPLLKLTSAVVDSQISCCKGDLCNAVVLAASSPWA
LCVQLLLSLGSVFLWALL

TABLE 3
SEQ ID NO: 5
>ly6-BIG1.3_DNA
CTGAAGTTTGTCTGTGCACTAGCACCCTGGAATGAGCAGTCTCCAGGC
CATGAAGACCTTGTCCCTGGTCCTGCTGGTGGCCCTGCTGAGCATGGA
GAGAGCTCAGGGTCTGCGCTGCTACAGATGCTTGGCGGTCTTGGAAGG
GGCCTCCTGCAGCGTGGTCTCGTGCCCCTTCCTGGATGGGGTCTGTGT
CTCCCAGAAAGTAAAGTGGGAGGGGAGAACAAGCTCCCCCTCCTCTCC
TGCCAGAAGGACGTCGGATTCCCCCTGCTGAAACTTACAAGTGCCGTT
GTGGACTCCCAGATCTCTTGCTGCAAGGGAGACCTCTGCAATGCGGTG
GTCCTGGCAGCCGGCAGCCCCTGGGCCCTGTGCGTACAGCTCCTGCTC
AGCCTGGGGTCAGTCTTCCTCTGGGCCCTGCTGTGAGGGCCTTTCCCG
CCCTCTCCCCCGCAGGCCTACCCTCTGTCCCTGTGCGTCACCAGCTGC
TTGGTTTTGAAGAGCTGC
SEQ ID NO: 6
>ly6-BIG1.3_Protein
MSSLQAMKTLSLVLLVALLSMERAQGLRCYRCLAVLEGASCSVVSCPF
LDGVCVSQKVKWEGRTSSPSSPARRTSDSPC

TABLE 4
SEQ ID NO: 7
>ly6-BIG1.4_DNA
CTGAAGTTTGTCTGTGCACTCCTTAANCTGGAATGAGCAGTCTCCAGGC
CATGAAGACCTTGTCCCTGGTCCTGCTGGTGGCCCTGCTGAGCATGGAG
AGAGGGTCTGCGCTGCTACAGATGCTTGGCGGTCTTGGAAGGGGCCTCC
TGCAGCGTGGTCTCGTGCCCCTTCCTGGATGGGGTCTGTGTCTCCCAGA
AAGTAAAGTGAGAGGGGAGAACAAGCTCTCCCTCCTCTCCTGCCAGAAG
GACGTCGGATTCCCCCTGCTGAAACTTACAAGTGCCGTTGTGGACTCCC
AGATCTCTTGCTGCAAGGGAGACCTCTGCAATGCGGTGGTCCTGGCAGC
CGGCAGCCCCTGGGCCCTGTGCGTACAGCTCCTGCTCAGCCTGGGGTCA
GTCTTCCTCTGGGCCCTGCTGTGAGGGCCTTTCCCGCCCTCTCCCCCGC
AGGCCTACCCTCTGTCCCTGTGCGTCACCAGCTGCTTGGTTTTGAAGAG
CTGC
SEQ ID NO: 8
>ly6-BIG1.4_Protein
MSSLQAMKTLSLVLLVALLSMERGSALLQMLGGLGRGLLQRGLVPLPGW
GLCLPESKVRGENKLSLLSCQKDVGFPLLKLTSAVVDSQISCCKGDLCN
AVVLAAGSPWALCVQLLLSLGSVFLWALL

TABLE 5
SEQ ID NO: 9
>ly6-BIG1.5_DNA
CTGAAGTTTGTCTGCGCACTAGCACCCTGGAATGAGCAGTCTCCAGGCC
ATGAAGACCTTGTCCCTGGTCCTGCTGGTGGCCCTGCTGAGCATGGAGA
GAGCTCAGGGTCTGCGCTGCTACAGATGCTTGGCGGTCTTGGAAGGGGC
CTCCTGCAGCGTGGTCTCGTGCCCCTTCCTGGATGGGGTCTGTGTCTCC
CAGAAAGTAAAGTGAGAGGGGAGAACAAGCTCTCCCTCCTCTCCTGCCA
GAAGGACGTCGGATTCCCCCTGCTGAAACTTACAGGTGCCGTTGTGGAC
TCCCAGATCTCTTGCTGCAAGGGAGACCTCTGCAATGCGGTGGTCCTGG
CAGCCGGCAGCCCCTGGGCCCTGTGCGTACAGCTCCTGCTCAGCCTGGG
GTCAGTCTTCCTCTGGGCCCTGCTGTGAGGGCCTTTCCCGCCCTCTCCC
CCGCAGGCCTACCCTCTGTCCCTGTGCGTCACCANCTGCTTGGTTTTGA
AGAGCTGCAATCGAA
SEQ ID NO: 10
>ly6-BIG1.5_Protein
MSSLQAMKTLSLVLLVALLSMERAQGLRCYRCLAVLEGASCSVVSCPFL
DGVCVSQKVK

TABLE 6
SEQ ID NO: 11
>ly6-BIG1.6_DNA
CTGAAGTTTGTCTGTGCACTGGCACCCTGGAATGAGCAGTCTCCAGGCC
ATGGAGACCTTGTCCCTGGTCCTGCTGGTGGCCCTGCTTGCCCTCTCTC
CCAGCTCAGGGTCTGCGCTGCTACAGATGCTTGGCGGTCTTGGAAGGGG
CCTCCTGCAGCGTGGTCTCGTGCCCCTTCCTGGATGGGGTCTGTGTCTC
CCAGAAAGTGAGCGTCTTTGGCAGTGAGTCCCTGGGGTGCCAGGGCAGA
GGGCAGGTTAAGTGCCGTTGTGGACTCCCAGATCTCTTGCTGCAAGGGA
GACCTCTGCAATGCGGTGGTCCTGGCAGCCGGCAGCCCCTGGGCCCTGT
GCGTACAGCTCCTGCTCAGCCTGGGGTCAGTCTTCCTCTGGGCCCCGCT
GTGAGGGCCTTTCCCGCCCTCTCCCCCGCAGGCCTACCCTCTGTCCCTG
TGCGTCACCAGCTGCTTGGTTTTGAAGAGCTG
SEQ ID NO: 12
>ly6-BIG1.6_Protein
MSSLQAMETLSLVLLVALLALSPSSGSALLQMLGGLGRGLLQRGLVPLP
GWGLCLPESERLWQ

TABLE 7
SEQ ID NO: 13
>ly6-BIG1.7_DNA
CTGAAGTTTGTCTGTGCACTAGCACCCTGGAATGAGCAGTCTCCAGGCC
ATGAAGACCTTGTCCCTGGTCCTGCTGGTGGCCCTTGCCCTCTCTCCCA
GCTCAGGGTCTGCGCTGCTACAGATGCTTGGCGGTCTTGGAAGGGGCCT
CCTGCAGCGTGGTCTCGTGCCCCTTCCTGGATGGGGTCTGTGTCTCCCA
GAAAGTGAGCGTCTTTGGCAGTGAGTCCCTGGGGTGCCAGGGCAGAGGG
CAGGTTCAGCTCCATGCAGGAGAGGCGCAGGCTGTGAGCATTCAGTGAG
TTACCTGCCTGGAAGAACAAGTGCCGTTGTGGACTCCCAGATCTCTTGC
TGCAAGGGAGACCTCTGCAATGCGGTGGTCCTGGCAGCCAGCAGCCCCC
TCTGTNCCCTGTGCGTCACCAGCTGCTTGGTTTTGAAGAGCTGCAATCG
AA
SEQ ID NO: 14
>ly6-BIG1.7_Protein
MSSLQAMKTLSLVLLVALALSPSSGSALLQMLGGLGRGLLQRGLVPLPG
WGLCLPESERLWQ

TABLE 8
SEQ ID NO: 15
>ly6-BIG1.8_DNA
CTGAAGTTTGTCTGTGCACTAGCACCCTGGAATGAGCAGTCTCCAGGCC
ATGAAGACCTTGTCCCTGGTCCTGCTGGTGGCCCTGCTGAGCATGGAGA
GAGCTCAGGGTCTGCGCTGCTACAGATGCTTGGCGGTCTTGGAAGGGGC
CTCCTGCAGCGTGGTCTCGTGCCCCTTCCTGGATGGGGTCTGTGTCTCC
CAGAAAGTGAGCGTCTTTGGCAGTAAAGTGAGAGGGGAGAACAAGCTCT
CCCTCCTCTCCTGCCAGAAGGACGTCGGATTCCCCCTGCTGAAACTTAC
AAGTGCCGTTGTGGACTCCCAGATCTCTTGCTGCAAGGGAGACCTCTGC
AATGCGGTGGTCCTGGCAGCCAGCAGCCCCTGGGCCCTGTGCGTACAGC
TCCTGCTCAGCCTGGGGTCAGTCTTCCTCTGGGCCCTGCTGTGAGGGCC
TTTCCCGCCCTCTCCCCCGCGGGCCTAGCCCTCTGTNCCCTGTGCGTCA
CCAGCTGCTTGGTTTGAAGAGCTGC
SEQ ID NO: 16
>ly6-BIG1.8_Protein
MSSLQAMKTLSLVLLVALLSMERAQGLRCYRCLAVLEGASCSVVSCPFL
DGVCVSQKVSVFGSKVRGENKLSLLSCQKDVGFPLLKLTSAVVDSQISC
CKGDLCNAVVLAASSPWALCVQLLLSLGSVFLWALL

TABLE 9
SEQ ID NO: 17
>ly6-BIG1.9_DNA
CTGAAGTTTGTCTGTGCACTAGCACCCTGGAATGAGCAGTCTCCAGGCC
ATGAAGACCTTGTCCCTGGTCCTGCTGGTGGCCCTGCTGAGCATGGAGA
GAGCTCAGGGTCTGCGCTGCTACAGATGCTTGGCGGTCTTGGAAGGGGC
CTCCTGCAGCGTGGTCTCGTGCCCCTTCCTGGATGGGGTCTGTGTCTCC
CAGAAAGTGAGCGTCTTTGGCAGTAAAGTGAGAGGGGAGAACAAGCTCT
CCCTCCTCTCCTGCCAGAAGGACGTCGGATTCCCCCTGCTGAAACTTAC
GAGTGCCGTTGTGGACTCCCAGATCTCTTGCTGCAAGGGAGACCTCTGC
AATGCGGTGGTCCTGGCAGCCAGCAGCCCCTGGGCCCTGTGCGTACAGC
TCCTGCTCAGCCTGGGGTCAGTCTTCCTCTGGGCCCTGCTGTGAGGGCC
TTTCCCGCCCTCTCCCCCGCGGGCCTACCCTCTGTCCCTGTGCGTCACC
AGCTGCTTGGTTTGAAGAGCTG
SEQ ID NO: 18
>ly6-BIG1.9_Protein
MSSLQAMKTLSLVLLVALLSMERAQGLRCYRCLAVLEGASCSVVSCPFL
DGVCVSQKVSVFGSKVRGENKLSLLSCQKDVGFPLLKLTSAVVDSQISC
CKGDLCNAVVLAASSPWALCVQLLLSLGSVFLWALL

TABLE 10
SEQ ID NO: 19
>ly6-BIG1.10_DNA
CTGAAGTTTGTCTGTGCACTGGCACCCTGGAATGAGCAGTCTCCAGGC
CATGAAGACCTTGTCCCTGGTCCTGCTGGTGGCCCTGCTGAGCATGGA
GAGAGGGTCTGCGCTGCTACAGATGCTTGGCGGTCTTGGAAGGGGCCT
CCTGCAGCGTGGTCTCGTGCCCCTTCCTGGATGGGGTCTGTGTCTCCC
AGAAAGTGAGCGTCTTTGGCAGTAAAGTGAGAGGGGAGAACAAGCTCT
CCCTCCTCTCCTGCCAGAAGGACGTCGGATTCCCCCTGCTGAAACTTA
CAAGTGCCGTTGTGGACTCCCAGATCTCTTGCTGCAAGGGAGACCTCC
GCAATGCGGTGGTCCTGGCAGCCGGCAGCCCCTGGGCCCTGTGCGTAC
AGCTCCTGCTCAGCCTGGGGTCAGTCTTCCTCTGGGCCCTGCTGTGAG
GGCCTTTCCCGCCCTCTCCCCCGCAGGCANTACCCTCTGTCCCTGTGC
GTCACCAGCTGCTTGGTTTTGAAGAGCTGC
SEQ ID NO: 20
>ly6-BIG1.10_Protein
MSSLQAMKTLSLVLLVALLSMERGSALLQMLGGLGRGLLQRGLVPLPG
WGLCLPESERLWQ

TABLE 11
SEQ ID NO: 21
>ly6-BIG1.11_DNA
TGAAGTTTGTCTGTGCACTGGCACCCTGGAATGAGCAGTCTCCAGGCC
ATGAAGACCTTGTCCCTGGTCCTGCTGGTGGCCCTGCTGAGCATGGAG
AGAGCTCAGGGTCTGCGCTGCTACAGATGCTTGGCGGTCTTGGAAGGG
GCCTCCTGCAGCGTGGTCTCGTGCCCCTTCCTAGATGGGGTCTGTGTC
TCCCAGAAAGTAAAGTGAGAGGGGAGAACAAGCTCTCCCTCCTCTCCT
GCCAGAAGGACGTCGGATTCCCCCTGCTGAAACTTACAAGTGCCGTTG
TGGACTCCCAGGTCTCTTGCTGCAAGGGAGACCTCTGCAATGCGGTGG
TCCTGGCAGCCGGCAGCCCCTGGGCCCTGTGCGTACAGCTCCTGCTCA
GCCTGGGGTCAGTCTTCCTCTGGGCCCTGCTGTGAGGGCCTTTCCCGC
CCACTCCCCCGCAGGCCTACCCTCTGTCCCTGTGCGTCACCAGCTGCT
TGGTTT
SEQ ID NO: 22
>ly6-BIG1.11_Protein
MSSLQAMKTLSLVLLVALLSMERAQGLRCYRCLAVLEGASCSVVSCPF
LDGVCVSQKVK

TABLE 12
SEQ ID NO: 23
>ly6-BIG1.12_DNA
CTGAAGTTTGTCTGTGCACTAGCACCCTGGAATGAGCAGTCTCCAGGC
CATGAAGACCTTGTCCCTGGTCCTGCTGGTGGCCCTGCTGAGCATGGA
GAGAGGTGAGAAGCAGAGGGGCCTTTAGAGGACTTTGTTCCAGCGCAC
TCCTGCTGCCCCGTGTGTGCTGGAACTAGTTGCAGGTGGGTGTGCTCG
GAAGGCGTGCCTGCTGGGGGTGGCGGGCTTCGGTGTTCCGGTGGCAGA
GGTGACTGGTGTGTTTGGTGCCTGCTCTGTGCTTGTTACCGCGCGTGC
TGGCTGTGCTCACTTCCGAGGACTCACTGAGTCCTGGGCACGTGTATG
CCTTTGGCATTGGGCAGTGGCTGCTGGTGCCTCTGGACAAAGAGGTGG
TGTTGGAGGGTTGCAGGCCACCAGTTGCAGGCTGCCAGTTGCAGGCAG
GTGTGTGGGGCTATTGCAAAGGTCCAGGTGGCAGGTTGGGACAAGGGT
GGTGGTGAGAGTGGGTGCCCTTGTGGGCATGGGACTCTCACCAGGGCA
TTGGTGTATGTCCTGGCATGTGCGTCACCAGCTGCTTGGTTTGAAGAG
CTGC
SEQ ID NO: 24
>ly6-BIG1.12_Protein
MSSLQAMKTLSLVLLVALLSMERGEKQRGL

TABLE 13
SEQ ID NO: 25
>ly6-BIG1.13_DNA
CTGAAGTTTGTCTGTGCACTGGCACCCTGGAATGAGCAGTCTCCAGGC
CATGAAGACCTTGTCCCTGGTCCTGCTGGTGGCCCTGCTGAGCATGGA
GAGGGCTCAGGGTCTGCGCTGCTACAGATGCTTGGCGGTCTTGGAAGG
GGCCTCCTGCAGCGTGGTCTCGTGCCCCTTCCTGGATGGGGTCTGTGT
CTCCCAGAAAGTAAAGTGAGAGGGGAGAACAAGCTCTCCCTCCTCTCC
TGCCAGAAGGACGTCGGATTCCCCCTGCTGAAACTTACAAGTGCCGTT
GTGGACTCCCAGATCTCTTGCTGCAAGGGAGACCTCTGCAATGCGGTG
GTCCTGGCAGCCGGCAGCCCCTGGGCCCTGTGCGTACAGCTCCTGCTC
AGCCTGGGGTCAGTCTTCCTCTGGGCCCTGCTGTGAGGGCCTTTCCCG
CCCTCTCCCCCGCAGGCCTACCCTCTGTCCCTGTGCGTCACCAGCTGC
TTGGTTTGAAGAGCTGC
SEQ ID NO: 26
>ly6-BIG1.13_Protein
MSSLQAMKTLSLVLLVALLSMERAQGLRCYRCLAVLEGASCSVVSCPF
LDGVCVSQKVK

TABLE 14
SEQ ID NO: 27
>ly6-BIG2_DNA
ATGCTGGGGCTGCTGGTGGCGTTGCTGGCCCTGGGGCTCGCTGTCTTT
GCGCTGCTGGACGTCTGGTACCTGGTGCGCCTTCCGTGCGCCGTGCTG
CGCGCGCGCCTGCTGCAGCCGCGCGTCCGTGACCTGCTAGCTGAGCAG
CGCTTCCCGGGCCGCGTGCTGCCCTCGGACTTGGACCTGCTGCTGCAC
ATGAACAACGCGCGCTACCTGCGCGAGGCCGACTTTGCGCGCGTCGCG
CACCTGACCCGCTGCGGGGTGCTCGGGGCGCTGAGGGAGTTGCGGGCG
CACACGGTGCTGGCGGCCTCGTGCGCGCGCCACCGCCGCTCGCTGCGC
CTGCTGGAGCCCTTCGAGGTGCGCACCCGCCTGCTGGGCTGGGACGAC
CGCGCGTTCTACCTGGAGGCGCGCTTTGTCAGCCTGCGGGACGGCTTC
GTGTGCGCGCTGCTGCGCTTCCGGCAGCACCTGCTGGGCACCTCACCC
GAGCGCGTCGTGCAGCACCTGTGCCAGCGCAGGGTGGAGCCCCCTGAG
CTGCCCGCTGATCTGCAGCACTGGATCTCCTACAACGAGGCCAGCAGC
CAGCTGCTCCGCATGGAGAGTGGGCTCAGTGATGTCACCAAGGACCAG
SEQ ID NO: 28
>ly6-BIG2_Protein
MLGLLVALLALGLAVFALLDVWYLVRLPCAVLRARLLQPRVRDLLAEQ
RFPGRVLPSDLDLLLHMNNARYLREADFARVAHLTRCGVLGALRELRA
HTVLAASCARHRRSLRLLEPFEVRTRLLGWDDRAFYLEARFVSLRDGF
VCALLRFRQHLLGTSPERVVQHLCQRRVEPPELPADLQHWISYNEASS
QLLRMESGLSDVTKDQ

TABLE 15
SEQ ID NO: 29
>ly6-BIG3_DNA
ATGCGGGGGACGCGGCTGGCGCTCCTGGCGCTGGTGCTGGCTGCCTGC
GGAGAGCTGGCGCCGGCCCTGCGCTGCTACGTCTGTCCGGAGCCCACA
GGAGTGTCGGACTGTGTCACCATCGCCACCTGCACCACCAACGAAACC
ATGTGCAAGACCACACTCTACTCCCGGGAGATAGTGTACCCCTTCCAG
GGGGACTCCACGGTGACCAAGTCCTGTGCCAGCAAGTGTAAGCCCTCG
GATGTGGATGGCATCGGCCAGACCCTGCCCGTGTCCTGCTGCAATACT
GAGCTGTGCAATGTAGACGGGGCGCCCGCTCTGAACAGCCTCCACTGC
GGGGCCCTCACGCTCCTCCCACTCTTGAGCCTCCGACTG
SEQ ID NO: 30
>ly6-BIG3_Protein
MRGTRLALLALVLAACGELAPALRCYVCPEPTGVSDCVTIATCTTNET
MCKTTLYSREIVYPFQGDSTVTKSCASKCKPSDVDGIGQTLPVSCCNT
ELCNVDGAPALNSLHCGALTLLPLLSLRL

TABLE 16
SEQ ID NO: 31
>ly6-BIG4_DNA
ATGAGGCTTCTCCTGGTGCTGCTGCTTCTGGCTGCTGTGTGTGCTGCC
CTGGCTCAGGCCCTGCACTGCCACGTGTGCTGCGGCCATGAGCACTGC
GAGTCCCTGGTGGAGTGTGCCCCCACTGACAAATACTGTGTGATCACA
CGGGCCACCAGCCCCGGTGGCATCCTGGTCATGAAGTCCTGCTCCCCG
ACGTGCCCCAACAGCACTGTGTCCTCCGACAGCCGCGCCCTCTCTGTG
TCCTGCTGCCAGGGTAGCCAGTGCAACCGCAGTGCAGCCGCAGGCCTG
GTGGGCAGCCCCGGGACCCTGTGGGCCAGCATCGCAGCCAGCCAGCTG
TGGGCCCTGCTGCAGGCAGCCCGC
SEQ ID NO: 32
>ly6-BIG4_Protein
MRLLLVLLLLAAVCAALAQALHCHVCCGHEHCESLVECAPTDKYCVIT
RATSPGGILVMKSCSPTCPNSTVSSDSRALSVSCCQGSQCNRSAAAGL
VGSPGTLWASIAASQLWALLQAAR

TABLE 17
SEQ ID NO: 33
>ly6-BIG5_DNA
ATGAAGGTCACTAGCCCCATGCTGCTGCTGGCTGAGGGCCAGGGCCTT
GAGTGCTTCCAGTGCTACGGTGTCCTGGACCCCAGCCTGTGTCACCCC
GTCTCCTATCCCATGCAGGCTCAAAGCTGCCCCTCCTCTGTGGTCACT
GGCACTATCGATGGTGAGTCCTGGGTGGGACCCAGCGTCTGTAGGCAG
GGCAGAAGCTCAGCTACACTAGCAAGGGCTGTGGCCCCACTCTGTGCC
CAGATTATGAACCTCACCCATCCTGTGGTCCCTGGAGGGTCTTACCCC
ACAGAAATTGAGGATAGACTGATTGACTCGAAGATTGAGAAGCTGGAC
ATGACCTGCTGTGAAAATAGCCTCCGTAACAAGGCGGCCACAGTGCGG
CGTGGCCTCTGGTGCCAGGCTGTCAGGGAGCTCCTGCTCAGCCTGAGC
CCCTTCCTCTGGGCTCTGCTG
SEQ ID NO: 34
>ly6-BIG5_Protein
MKVTSPMLLLAEGQGLECFQCYGVLDPSLCHPVSYPMQAQSCPSSVVT
GTIDGESWVGPSVCRQGRSSATLARAVAPLCAQIMNLTHPVVPGGSYP
TEIEDRLIDSKIEKLDMTCCENSLRNKAATVRRGLWCQAVRELLLSLS
PFLWALL

TABLE 18
SEQ ID NO: 35
>ly6-BIG6_DNA
ATGGAGAGGCTCGTCCTAACCCTGTGCACCCTCCCGCTGGCTGTGGCG
TCTGCTGGCTGCGCCACGACGCCAGCTCGCAACCTGAGCTGCTACCAG
TGCTTCAAGGTCAGCAGCTGGACGGAGTGCCCGCCCACCTGGTGCAGC
CCGCTGGACCAAGTCTGCATCTCCAACGAGGTGGTCGTCTCTTTTAGG
TCAGAACAAGTGACAGAGGTCACCAGGGGCTGCACCAACAACCGCATC
GTCTCGGCCCGTCCCGGCTGGGAGGAGTTCACCTGGGACAGCATCCTC
TGTGCCAGCGTCTTGTGCTGTTTGGAGACCCTGGGTAACCGGGAAGCC
ATGGCAGGCAGCGCTGCCCAGGCCCTGCAAGGGGGCTGCAGCTCACCC
AGTGGAGGGCGTCCCCTGACAAAGCCGCCCCTCTGTGCTGTGAGGTGG
GAGGAGCCTCTGCCTGTCTACCGGCCCCAGATTCCACGCCCATCGGGG
AAGCCCGGCAAAGGCACCAGCACTGGGAATGTGCCCCAGCAAACAGTG
AGCAACGAGGAGGCTGACGGTAGTGAGGTCACGGCACGCACCTTGCTG
ATGACCGGGGTTCAGCCAGACGTAACTCTGGGAAAACAGACTGAGCTC
AGCCCTTTCAGCCAGCGGCCATTCAGCTTTTCTGACCATGGCCCATAT
CAACAAACACAATATGGTACCCACTCTTGCTGGCACCAGGACACTGAG
ACGGTCCAGGAAACACGGCAAGCATACGTGTGCACCACACTGCTCGTC
CCGTCCAGCTGTGGCCATGCTGAAAATTGCAATGGGCCACTGGAAGAC
AGGTTATTCAGGCAGGACACCCAGAGGAGCTTCCAGCCCGCAGTTTCA
GTGGTGCCCAGCAAACAGCTCCTAGTGGCCTCTGAGGGCCTTGCCAGC
GTGGACAGCTCCTTGCCAGTGGGACGGTTCCTTGCCAGTGTGGACGGC
TCCTGCTGCAGCTCCCAAAGGGCGCTCTCCTGCCAGCCTTGGCTGAGG
CGCTGCTGCTTCAGTAGGGTCC
SEQ ID NO: 36
>ly6-BIG6_Protein
MERLVLTLCTLPLAVASAGCATTPARNLSCYQCFKVSSWTECPPTWCS
PLDQVCISNEVVVSFRSEQVTEVTRGCTNNRIVSARPGWEEFTWDSIL
CASVLCCLETIGNREAMAGSAAQALQGGCSSPSGGRPLTKPPLCAVRW
EEPLPVYRPQIPRPSGKPGKGTSTGNVPQQTVSNEEADGSEVTARTLL
MTGVQPDVTLGKQTELSPFSQRPFSFSDHGPYQQTQYGTHSCWHQDTE
TVQETRQAYVCTTLLVPSSCGHAENCNGPLEDRLFRQDTQRSFQPAVS
VVPSKQLLVASEGLASVDSSLPVGRFLASVDGSCCSSQRALSCQPWLR
RCCFSRV

TABLE 19
SEQ ID NO: 37
>ly6-BIG7_DNA
ATGAAGGCGCTCGGGGCTGTCCTGCTTGCCCTCTTGCTGTGCGGGCGG
CCAGTGCTGCTGCGGTGCTACACCTGCAAGTCCCTGCCCAGGGACGAG
CGCTGCAACCTGACGCAGAACTGCTCACATGGCCAGACCTGCACAACC
CTCATTGCCCACGGGAACACCGAGTCAGGCCTCCTGACCACCCACTCC
ACGTGGTGCACAGACAGCTGCCAGCCCATCACCAAGACGGTGGAGGGG
ACCCAGGTGACCATGACCTGCTGCCAGTCCAGCCTGTGCAATGTCCCA
CCCTGGCAAAGCTCCCGAGTCCAGGACCCAACAGGCAAGGGGGCAGGC
GGCCCCCGGGGCAGCTCCGAAACTGTGGGCGCAGCCCTCCTGCTCAAC
CTCCTTGCCGGCCTTGGAGCAATGGGGGCCAGGAGACCC
SEQ ID NO: 38
>ly6-BIG7_Protein
MKALGAVLLALLLCGRPVLLRCYTCKSLPRDERCNLTQNCSHGQTCTT
LIAHGNTESGLLTTHSTWCTDSCQPITKTVEGTQVTMTCCQSSLCNVP
PWQSSRVQDPTGKGAGGPRGSSETVGAALLLNLLAGLGAMGARRP

TABLE 20
Ly6-BIG1 Fc1 (SEQ ID NO: _):
MKTLSLVLLVALLSMERAQGLRCYRCLAVLEGASCSVVSCPFLDGVC
VSQKVSVFGSKVRGENKLSLLSCQKDVGFPLLKLTSAVVDSQISCCKG
DLCNAVVLAASENLYFQGASQEPKSSDKTHTSPPSPAPELLGGPSVFLF
PPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALH
NHYTQKSLSLSPGK*

TABLE 21
Ly6-BIG1 Fc2 (SEQ ID NO: _):
MGSLQAMKTLSLVLLVALLSMERAQGLRCYRCLAVLEGASCSVVSCP
FLDGVCVSQKVSVFGSKVRGENKLSLLSCQKDVGFPLLKLTSAVVDS
QISCCKGDLCNAVVLAASSPWALCVQLLLSLGENLYFQGASQEPKSSD
KTHTSPPSPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVD
KSRWQQGNVFSCSVMHEALHNHYTQKSLSLSPGK*

TABLE 22
Ly6 domains of Ly6-BIG proteins of the invention

| Protein | Ly6 domain | Sequence |
|---|---|---|
| ly6-BIG1.1 | Amino acids | LRCYRCLAVLEGASCSVVSCPFLDGVCVSQKVSVFGSKVRGE |
| | 27-118 of | NKLSLLSCQKDVGFPLLKLTSAVVDSQISCCKGDLCNAVVLAA |
| | SEQ ID NO: 2 | SSPWALC |
| ly6-BIG1.2 | 27-97 of | LRCYRCLAVLEGASCSVVSCPFLDGVCVSQKKDVGFPLLKLTS |
| | SEQ ID NO: 4 | AVVDSQISCCKGDLCNAVVLAASSPWALC |
| ly6-BIG1.8 | 27-118 of | LRCYRCLAVLEGASCSVVSCPFLDGVCVSQKVSVFGSKVRGE |
| | SEQ ID NO: 16 | NKLSLLSCQKDVGFPLLKLTSAVVDSQISCCKGDLCNAVVLAA |
| | | SSPWALC |
| ly6-BIG1.9 | 27-118 of | LRCYRCLAVLEGASCSVVSCPFLDGVCVSQKVSVFGSKVRGE |
| | SEQ ID NO: 18 | NKLSLLSCQKDVGFPLLKLTSAVVDSQISCCKGDLCNAVVLAA |
| | | SSPWALC |
| ly6BIG3_ly6 | 23-101 of | LRCYVCPEPTGVSDCVTIATCTTNETMCKTTLYSREIVYPFQG |
| | SEQ ID NO: 30 | DSTVTKSCASKCKPSDVDGIGQTLPVSCCNTELCNVDGAPALN |
| | | SLHCGA |
| ly6BIG4_ly6 | 21-90 of | LHCHVCCGHEHCESLVECAPTDKYCVITRATSPGGILVMKSCS |
| | SEQ ID NO: 32 | PTCPNSTVSSDSRALSVSCCQGSQCNRSAAAGLVGSPG |
| ly6BIG5_ly6 | 16-123 of | LECFQCYGVLDPSLCHPVSYPMQAQSCPSSVVTGTIDGESWV |
| | SEQ ID NO: 34 | GPSVCRQGRSSATLARAVAPLCAQIMNLTHPVVPGGSYPTEIE |
| | | DRLIDSKIEKLDMTCCENSLRNK |
| ly6BIG6_ly6 | 28-110 of | LSCYQCFKVSSWTECPPTWCSPLDQVCISNEVVVSFRSEQVT |
| | SEQ ID NO: 36 | EVTRGCTNNRIVSARPGWEEFTWDSILCASVLCCLETLGNREA |
| | | MAGSAAQAL |
| ly6BIG7_ly6 | 20-95 of | LRCYTCKSLPRDERCNLTQNCSHGQTCTTLIAHGNTESGLLTT |
| | SEQ ID NO: 38 | HSTWCTDSCQPITKTVEGTQVTMTCCQSSLCNVPPWQSSRV |
| | | QD |

TABLE 23
Results of PCR on cDNA

| | | cDNA source | | | |
|---|---|---|---|---|---|
| | PCR | Bone | | | |
| Target | primers | marrow | Fetal brain | Spleen | Adult brain |
| BIG 1 ¹ | PCf2/395r | + | ++ | + | +/− |
| BIG 2 | 8/567 | − | − | − | − |
| | 8/612 | − | − | − | − |
| BIG 3 | 35/363 | +++ | − | ++ | − |
| | 35/375 | +++ | − | + | − |
| BIG 4 | 7/334 | − | − | − | − |
| | 25/334 | − | − | − | − |
| BIG 5 | 3/358 | − | − | − | − |
| | 17/358 | − | − | − | − |
| BIG 6 | 6/959 | − | − | − | − |
| | 41/959 | − | − | − | − |
| BIG 7 ² | 25/365 | − | +++ | +++ (4 ex) | + |
| | 25/394 | − | +++ | +++ (4 ex) | + |

PCR products of correct or incorrect sizes were cloned and sequenced for all targets, from spleen reactions only. Tissues other than spleen are shown as positive based on correct predicted product size.
¹ Slightly shorter splice variant is detected in some samples.
² BIG 7 results ftom spleen are shown with (4 ex) notation because these products had an extra exon in addition to the predicted 3, thus having similar composition to NCBI entry (NP_835466/LOC338328 high density lipoprotein-binding protein) at this site.

1. An isolated polynucleotide comprising a nucleic acid at least 85%, 90% or 95% identical to a nucleotide sequence in SEQ ID NOs: 1, 3, 5, 7, 9, 11, 13, 15, 17, 19, 21, 23, 25, 27, 29, 31, 33, 35, or 37.

2. An isolated polynucleotide comprising a nucleic acid encoding a polypeptide at least 85%, 90% or 95% identical to the amino acid sequence in SEQ ID NO:2, 4, 6, 8, 10, 12, 14, 16, 18, 20, 22, 24, 26, 28, 30, 32, 34, 36, or 38.

3. A fragment of the polynucleotide of claim 1 or claim 2.

4. A polypeptide encoded by the polynucleotide of any of claims 1-3.

5. The polypeptide of claim 4, which is 100% identical to the amino acid sequence in SEQ ID NO:2, 4, 6, 8, 10, 12, 14, 16, 18, 20, 22, 24, 26, 28, 30, 32, 34, 36, or 38.

6. A host cell comprising the polynucleotide of any of claims 1-3.

7. An antibody that specifically binds the polypeptide of claim 4 or claim 5.

8. A composition comprising the polynucleotide of any of claims 1-3, the polypeptide of claim 4 or claim 5, or the antibody of claim 7.

9. A method of treating autoimmune disorders or cancer in a patient in need thereof, comprising administering the antibodies of claim 7 to said patient or to cells derived from said patient.
